SEQUENCE LISTING (145 sequences)

SEQ ID NO: 1 - DNA, 819 bp, Staphylococcus aureus
atgtatttacttacctccaattataattgtaccggttcaatttgtaaacgccgatacaattataatattttgtgctataataattacagacaaagtgaaaacgaggacagaatattgttaaagtatgaacatattgctaagcaacttaatgcgtttatacatcaatctaatttcaaacccggtgataaattgccaagcgtgacgcaattaaaagaacgttatcaagtaagtaagagtactatcattaaagcattaggcttattggaacaagatggtttgatctatcaagcacaaggcagtggtatttatgtgagaaatattgctgatgccaatcgtatcaacgtctttaagactaatggtttctctaaaagtttaggtgaacaccgaatgacaagtaaggtacttgtttttaaggagattgcaacgccacctaaatctgtacaagatgagctccaattaaatgcagatgataccgtctactatttagagcgattaagattcgtggacgatgatgttttatgtatcgaatattcttattatcataaagaaatcgtgaaatatttaaatgatgatattgctaagggctctatcttcgactatttagaatcaaacatgaaacttcgtattggtttttcagatattttctttaatgtagatcaactcacttcaagtgaagcttcattactacaattgtctacaggtgaaccatgtttacgttaccaccagactttttatacaatgactggcaaaccctttgattcatctgacatcgtatttcattatcgtcatgcacagttttatattcctagtaaaaagtaa

SEQ ID NO: 2 - protein, 272 aa, Staphylococcus aureus
MYLLTSNYNCTGSICKRRYNYNILCYNNYRQSENEDRILLKYEHIAKQLNAFIHQSNFKPGDKLPSVTQLKERYQVSKSTIIKALGLLEQDGLIYQAQGSGIYVRNIADANRINVFKTNGFSKSLGEHRMTSKVLVFKEIATPPKSVQDELQLNADDTVYYLERLRFVDDDVLCIEYSYYHKEIVKYLNDDIAKGSIFDYLESNMKLRIGFSDIFFNVDQLTSSEASLLQLSTGEPCLRYHQTFYTMTGKPFDSSDIVFHYRHAQFYIPSKK

SEQ ID NO: 3 - DNA, 828 bp, Staphylococcus aureus
atggcactttatggatttgcccaaggacttattcaagaagcaggaattagaattaaacaattgatggagcaaaatttaacaattgaaacaaagtcaaatccgaatgaccttgttacaaatgtagataaagcaacagaagatttcatttttgatacaattttagaaacatatcccaatcatcaagtattaggtgaagaagggcatggtcatgacatcgatacttccaaaggtacggtatggattgttgacccaatagacggtacattgaattttgttcatcaacaagaaaatttcgcaatttcaattggtatttatatcgatggtaaaccttatgcaggttttgtatatgatgttatggctgatgtcttatatcatgctaaagtaggggaaggtgcatatcgtggtagccaacccttgaaaccattgaatgattctaatctaagacaaagcattattgggatcaatccgaactggttaactaaaccaattttaggagaaatctttaaagaaattgttaatgattctagaagtgcaagggcatatggtagtgcagcgcttgaaatcgtttcagttgctacaggtaatttagaagcatatatgacgccaagacttcaaccatgggattttgctggcggattggttattttatatgaagtaaatggacaagcttccaatttactaggaggaccattaacaattagtggtccaaattcaatcttagttggaaatcgtggtctccatcaagaaattagcaatgattatttagagccccaccatgatgcgttaatacaattacatgaacaacgatttaaaagaaaatcaaaataa

SEQ ID NO: 4 - protein, 275 aa, Staphylococcus aureus
MALYGFAQGLIQEAGIRIKQLMEQNLTIETKSNPNDLVTNVDKATEDFIFDTILETYPNHQVLGEEGHGHDIDTSKGTVWIVDPIDGTLNFVHQQENFAISIGIYIDGKPYAGFVYDVMADVLYHAKVGEGAYRGSQPLKPLNDSNLRQSIIGINPNWLTKPILGEIFKEIVNDSRSARAYGSAALEIVSVATGNLEAYMTPRLQPWDFAGGLVILYEVNGQASNLLGGPLTISGPNSILVGNRGLHQEISNDYLEPHHDALIQLHEQRFKRKSK

SEQ ID NO: 5 - DNA, 543 bp, Staphylococcus aureus
atgggattcaaaaacaatttaacatcaaatttaacaaataaaatcggtaattcagtctttaaaatagaaaatgttgacggaaaaggtgcaatgccaacgacgattcaagaattgagagaaagacgacaacgtgctgaagcaattgtaaagagaaagtctttaatgtcatcaacaatgagcgttgttccaattccgggtttagattttggtgttgatttaaaattaatgaaagatattatcgaagatgttaataaaatttatggtttagatcataagcaagttaatagccttggggatgatgtgaaagaaagaattatgtctgcagcagcaattcaaggtagtcaatttattggtaaaagaatttcaaatgcatttttaaaaattgtaattagagatgtagctaaacgtactgctgcaaaacaaacaaaatggtttcctgttgtaggacaagctgtgtctgcatctattagttactattttatgaataaaattggaaaagatcacattcaaaaatgcgaaaatgttattaaaaatgtcatgtag

SEQ ID NO: 6 - protein, 180 aa, Staphylococcus aureus
MGFKNNLTSNLTNKIGNSVFKIENVDGKGAMPTTIQELRERRQRAEAIVKRKSLMSSTMSVVPIPGLDFGVDLKLMKDIIEDVNKIYGLDHKQVNSLGDDVKERIMSAAAIQGSQFIGKRISNAFLKIVIRDVAKRTAAKQTKWFPVVGQAVSASISYYFMNKIGKDHIQKCENVIKNVM

SEQ ID NO: 7 - DNA, 936 bp, Staphylococcus aureus
gtgtttcatcatatcagcgttatgttaaacgaaaccattgattatttaaatgtaaaagaaaatggtgtgtacattgactgtacgctaggtggagcgggacatgccctttatttactaaatcaattaaatgacgacggaagattaatagcaatcgatcaagaccaaactgcaattgataatgctaaagaggtattaaaggatcatttgcataaggtgacttttgttcatagcaacttccgtgaattaactcaaatattaaaagacttaaacattgaaaaagtagatggaatttattacgacttgggtgtttcaagcccacaactcgacattccagaacgaggattcagttatcaccatgacgcaacattagacatgcgtatggaccaaacacaagaactaacagcatatgaaattgttaacaattggtcatatgaagcgttagtgaagattttttatcgctatggcgaggagaaattttcaaaacagatagctcgaagaatcgaagcacatcgcgaacaacaaccaataacaacaacattagaattagttgacattataaaagaaggtattcctgcaaaagcaagaagaaaaggcggacatcctgcaaaacgagtatttcaagcactacgaattgcagtaaacgatgaattgtcagcttttgaagattcaatagaacaagcgattgaattagtgaaagtagatggcaggatttcggtaatcactttccattctttagaagatcgtttatgtaaacaggtgttccaagaatatgaaaaaggtccagaggtaccaagaggattaccagttataccagaagcatatacacctaagttaaagcgtgttaatcgtaaaccgattaccgctacagaagaagatttagatgacaataacagagcacgaagcgcgaaattacgtgtagctgaaatacttaaataa

SEQ ID NO: 8 - protein, 311 aa, Staphylococcus aureus
VFHHISVMLNETIDYLNVKENGVYIDCTLGGAGHALYLLNQLNDDGRLIAIDQDQTAIDNAKEVLKDHLHKVTFVHSNFRELTQILKDLNIEKVDGIYYDLGVSSPQLDIPERGFSYHHDATLDMRMDQTQELTAYEIVNNWSYEALVKIFYRYGEEKFSKQIARRIEAHREQQPITTTLELVDIIKEGIPAKARRKGGHPAKRVFQALRIAVNDELSAFEDSIEQAIELVKVDGRISVITFHSLEDRLCKQVFQEYEKGPEVPRGLPVIPEAYTPKLKRVNRKPITATEEDLDDNNRARSAKLRVAEILK

SEQ ID NO: 9 - DNA, 969 bp, Staphylococcus aureus
atgataaataatcatgaattactaggtattcaccatgttactgcaatgacagatgatgcagaacgtaattataaattttttacagaagtactaggcatgcgtttagttaaaaagacagtcaatcaagatgatatttatacgtatcatactttttttgcagatgatgtaggttcggcaggtacagacatgacgttctttgattttccaaatattacaaaagggcaggcaggaacaaattccattacaagaccgtcttttagagtgcctaacgatgacgcattaacatattatgaacagcgctttgatgagtttggtgttaaacacgaaggtattcaagaattatttggtaaaaaagtgttgccatttgaagaagtcgatggccaagtgtatcaattaatttcagatgagttaaatgaaggggtagcacctggtgtaccttggaagaatggaccggttccagtagataaagcgatttatggattaggccccattgaaattaaagtaagttattttgacgactttaaaaatattttagagactgtttacggtatgacaactattgcgcatgaagataatgtcgcattacttgaagttggcgaaggaggcaatggtggccaggtaatcttaataaaagatgataaagggccagcagcacgtcaaggttatggtgaggtacatcatgtgtcatttcgtgtgaaagatcatgatgcaatagaagcgtgggcaacgaaatataaagaggtaggtattaataactcaggcatcgttaatcgtttctattttgaagcattatatgcacgtgtggggcatattttaatagaaatttcaacagatggaccaggatttatggaagatgaaccttatgaaacattaggcgaagggttatccttaccaccatttttagaaaataaaagagaatatattgaatcggaagttagaccttttaatacgaagcgtcaacatggttaa

SEQ ID NO: 10 - protein, 322 aa, Staphylococcus aureus
MINNHELLGIHHVTAMTDDAERNYKFFTEVLGMRLVKKTVNQDDIYTYHTFFADDVGSAGTDMTFFDFPNITKGQAGTNSITRPSFRVPNDDALTYYEQRFDEFGVKHEGIQELFGKKVLPFEEVDGQVYQLISDELNEGVAPGVPWKNGPVPVDKAIYGLGPIEIKVSYFDDFKNILETVYGMTTIAHEDNVALLEVGEGGNGGQVILIKDDKGPAARQGYGEVHHVSFRVKDHDAIEAWATKYKEVGINNSGIVNRFYFEALYARVGHILIEISTDGPGFMEDEPYETLGEGLSLPPFLENKREYIESEVRPFNTKRQHG

SEQ ID NO: 11 - DNA, 1100 bp, Staphylococcus aureus
gggacatttttaaatcatgcatgcgtatcttaaaagagtccattattgtggcatttgcctttgttggtgttgtcgttggtgccggctttgctactggtcaagaaattttccagtttttcacaagtcatggcgcatatagcatttcaggcattattgtaacaggactattgattactttaggtggaatggttgtcatgcatacaggtcatcatctaaagtccagaaatcattctgattcaattaactatttcttatacccctctattgcaagaggttttgatattattttaacaatgtttatgttgtctttagctattattatgactgcaggtggtgcgtcaaccattcatcaaagtttcaacttaccgtattggctgagcgcactcatattagtcgcctttattttagcaacactgtttctaaaattcgatcgtttaattgctgtgcttggcggtgttaccccatttttaattgcgattgtcattatgattgcggtctactatttcacaacaagtcatcttgattttactgccgctaataatgatgctcagattcataagcagaaatcattatcacctggatggtggtttgatgcgattaactatgcaagcttgcaaattgctgctgccttcagcttcttatcagtgatgggtagtaaagttaaatatcgtgactcaacgttatacgggggcttgattggcggtttaatcattacatttttactcatgatgattaatctaggtttaatttctcaattcgataaaattaaacacgtagatctacctacattaaaattagcgacacaaatgtctccgtcaattggtattattatgtctgtcattatgatacttgtcatctacaatactgttgttggattaatgtatgcatttgcgtcacgtttcagcgttccattcagcagacgttacttcatcattattattacaatggctgtcatcacttatattagtacatttatcggtttcatttcattaattggaaaagtattccctattatgggattgttcggtttcatcttactcatacctgtactctataaaggtttaattaagcgtattaccggcaaatctcatatcgattaa

SEQ ID NO: 12 - protein, 359 aa, Staphylococcus aureus
MRILKESIIVAFAFVGVVVGAGFATGQEIFQFFTSHGAYSISGIIVTGLLITLGGMVVMHTGHHLKSRNHSDSINYFLYPSIARGFDIILTMFMLSLAIIMTAGGASTIHQSFNLPYWLSALILVAFILATLFLKFDRLIAVLGGVTPFLIAIVIMIAVYYFTTSHLDFTAANNDAQIHKQKSLSPGWWFDAINYASLQIAAAFSFLSVMGSKVKYRDSTLYGGLIGGLIITFLLMMINLGLISQFDKIKHVDLPTLKLATQMSPSIGIIMSVIMILVIYNTVVGLMYAFASRFSVPFSRRYFIIIITMAVITYISTFIGFISLIGKVFPIMGLFGFILLIPVLYKGLIKRITGKSHID

SEQ ID NO: 13 - DNA, 774 bp, Staphylococcus aureus
atgttaatcgatacacatgtccatttaaatgatgagcaatacgatgatgatttgagtgaagtgattacacgtgctagagaagcaggtgttgatcgtatgtttgtagttggttttaacaaatcgacaattgaacgcgcgatgaaattaatcgatgagtatgattttttatatggcattatcggttggcatccagttgacgcaattgattttacagaagaacacttggaatggattgaatctttagctcagcatccaaaagtgattggtattggtgaaatgggattagattatcactgggataaatctcctgcagatgttcaaaaggaagtttttagaaagcaaattgctttagctaagcgtttgaagttaccaattatcattcataaccgtgaagcaactcaagactgtatcgatatcttattggaggagcatgctgaagaggtaggcgggattatgcatagctttagtggttctccagaaattgcagatattgtaactaataagctgaatttttatatttcattaggtggacctgtgacatttaaaaatgctaaacagcctaaagaagttgctaagcatgtgtcaatggagcgtttgctagttgaaaccgatgcaccgtatctttcgccacatccgtatagagggaagcgaaatgaaccggcgagagtaactttagtagctgaacaaattgctgaattaaaaggcttatcttatgaagaagtgtgcgaacaaacaactaaaaatgcagagaaattgtttaatttaaattcataa

SEQ ID NO: 14 - protein, 257 aa, Staphylococcus aureus
MLIDTHVHLNDEQYDDDLSEVITRAREAGVDRMFVVGFNKSTIERAMKLIDEYDFLYGIIGWHPVDAIDFTEEHLEWIESLAQHPKVIGIGEMGLDYHWDKSPADVQKEVFRKQIALAKRLKLPIIIHNREATQDCIDILLEEHAEEVGGIMHSFSGSPEIADIVTNKLNFYISLGGPVTFKNAKQPKEVAKHVSMERLLVETDAPYLSPHPYRGKRNEPARVTLVAEQIAELKGLSYEEVCEQTTKNAEKLFNLNS

SEQ ID NO: 15 - DNA, 2123 bp, Staphylococcus aureus
atgataatatattggtgtatgacagttaatggagggaacgaaatgaaagctttattacttaaaacaagtgtatggctcgttttgctttttagtgtaatgggattatggcaagtctcgaacgcggctgagcagcatacaccaatgaaagcacatgcagtaacaacgatagacaaagcaacaacagataagcaacaagtaccgccaacaaaggaagcggctcatcattctggcaaagaagcggcaaccaacgtatcagcatcagcgcagggaacagctgatgatacaaacagcaaagtaacatccaacgcaccatctaacaaaccatctacagtagtttcaacaaaagtaaacgaaacacgcgacgtagatacacaacaagcctcaacacaaaaaccaactcacacagcaacgttcaaattatcaaatgctaaaacagcatcactttcaccacgaatgtttgctgctaatgcaccacaaacaacaacacataaaatattacatacaaatgatatccatggccgactagccgaagaaaaagggcgtgtcatcggtatggctaaattaaaaacagtaaaagaacaagaaaagcctgatttaatgttagacgcaggagacgccttccaaggtttaccactttcaaaccagtctaaaggtgaagaaatggctaaagcaatgaatgcagtaggttatgatgctatggcagtcggtaaccatgaatttgactttggatacgatcagttgaaaaagttagagggtatgttagacttcccgatgctaagtactaacgtttataaagatggaaaacgcgcgtttaagccttcaacgattgtaacaaaaaatggtattcgttatggaattattggtgtaacgacaccagaaacaaagacgaaaacaagacctgaaggcattaaaggcgttgaatttagagatccattacaaagtgtgacagcggaaatgatgcgtatttataaagacgtagatacatttgttgttatatcacatttaggaattgatccttcaacacaagaaacatggcgtggtgattacttagtgaaacaattaagtcaaaatccacaattgaagaaacgtattacagttattgatggtcattcacatacagtacttcaaaatggtcaaatttataacaatgatgcattggcacaaacaggtacagcacttgcgaatatcggtaagattacatttaattatcgcaatggagaggtatcgaatattaaaccgtcattgattaatgttaaagacgttgaaaatgtaacaccgaacaaagcattagctgaacaaattaatcaagctgatcaaacatttagagcacaaactgcagaggtaattattccaaacaataccattgatttcaaaggagaaagagatgacgttagaacgcgtgaaacaaatttaggaaacgcgattgcagatgctatggaagcgtatggcgttaagaatttctctaaaaagactgactttgccgtgacaaatggtggaggtattcgtgcctctatcgcaaaaggtaaggtgacacgctatgatttaatctcagtattaccatttggaaatacgattgcgcaaattgatgtaaaaggttcagacgtctggacggctttcgaacatagtttaggcgcaccaacaacacaaaaggacggtaagacagtgttaacagcgaatggcggtttactacatatctctgattcaatccgtgtttactatgatataaataaaccgtctggcaaacgaattaatgctattcaaattttaaataaagagacaggtaagtttgaaaatattgatttaaaacgtgtatatcacgtaacgatgaatgacttcacagcatcaggtgggacggatatagtatgttcggtggtcctagagaagaaggtatttcattagatcaagtactagcaagttatttaaaaacagctaacttagctaagtatgatacgacagaaccacaacgtatgttattaggtaaaccagcagtaagtgaacaaccagctaaaggacaacaaggtagcaaaggtagtaagtctggtaaagatacacaaccaattggtgacgacaaagtgatggatccagcgaaaaaaccagctccaggtaaagttgtattgttgtag

SEQ ID NO: 16 - protein, 707 aa, Staphylococcus aureus
MIIYWCMTVNGGNEMKALLLKTSVWLVLLFSVMGLWQVSNAAEQHTPMKAHAVTTIDKATTDKQQVPPTKEAAHHSGKEAATNVSASAQGTADDTNSKVTSNAPSNKPSTVVSTKVNETRDVDTQQASTQKPTHTATFKLSNAKTASLSPRMFAANAPQTTTHKILHTNDIHGRLAEEKGRVIGMAKLKTVKEQEKPDLMLDAGDAFQGLPLSNQSKGEEMAKAMNAVGYDAMAVGNHEFDFGYDQLKKLEGMLDFPMLSTNVYKDGKRAFKPSTIVTKNGIRYGIIGVTTPETKTKTRPEGIKGVEFRDPLQSVTAEMMRIYKDVDTFVVISHLGIDPSTQETWRGDYLVKQLSQNPQLKKRITVIDGHSHTVLQNGQIYNNDALAQTGTALANIGKITFNYRNGEVSNIKPSLINVKDVENVTPNKALAEQINQADQTFRAQTAEVIIPNNTIDFKGERDDVRTRETNLGNAIADAMEAYGVKNFSKKTDFAVTNGGGIRASIAKGKVTRYDLISVLPFGNTIAQIDVKGSDVWTAFEHSLGAPTTQKDGKTVLTANGGLLHISDSIRVYYDINKPSGKRINAIQILNKETGKFENIDLKRVYHVTMNDFTASGGDGYSMFGGPREEGISLDQVLASYLKTANLAKYDTTEPQRMLLGKPAVSEQPAKGQQGSKGSKSGKDTQPIGDDKVMDPAKKPAPGKVVLL

SEQ ID NO: 17 - DNA, 1482 bp, Staphylococcus aureus
atgcgatttacattttcaaacgatttaggaacgttatttactattattttagccattggattcatcattaatttagtattggcttttattattatctttttagaaagaaataggcgtacagcgagttcaacttgggcatggctatttgtactttttgtcttaccattgattggttttattctttacttgttttttggtagaaccgtttcggcacgcaaattgaataaaaacaatggtaacgtgttaacggatttcgatggacttttaaaacaacaaatagaaagctttgataaaggtaattatggtactgataacaaacaagttcaaaaacatcatgatttagtacgtatgcttttgatggatcaagatggttttttaactgaaaataataaagttgatcatttcattgatggaaatgatttatatgatcaagttttaaaagatattaaaaatgcaaaagaatatatccatttagagtactatactttcgctttagatggtttaggtaaaagaattttacatgctttagaagaaaaattgaaacaaggtctagaagtaaaaatattatatgatgatgttggatctaaaaatgttaagatggcaaattttgatcattttaaatcgttaggtggagaagttgaagcattttttgcttcaaaattaccgttattgaatttcagaatgaataatagaaatcatagaaaaatcatcgtaatcgatggtcaactaggttatgtcggaggatttaacattggtgatgaatatctaggattaggaaaattaggatattggagagatacgcatttacgtatacaaggggatgcggttgatgcactgcagttgcgatttattttagactggaattcgcaagcgcaccgtccacaatttgaatatgatgttaagtatttccctaaaaagaacggaccattgggcaattcaccaattcaaatagctgcaagtggcccggctagtgactggcatcaaattgaatacggttatacaaaaatgattatgagtgcaaagaaatctgtatatttacaatcaccatatttcattccggataattcatatataaatgccattaaaattgctgctaaatcaggtgtagatgtacatttaatgattccatgtaagccagatcatccattagtatattgggcgacattttcaaatgcctctgacttattatcaagtggtgttaaaatttatacgtatgaaaatggatttatacattctaaaatgtgcttaattgatgatgaaatcgtatcagtgggcacagcaaatatggactttagaagttttgaattaaattttgaagtaaatgcctttgtatatgatgaaaatcttgctaaagatttaagggtggcttatgaacatgatattacaaaatcaaaacaactaaccaaagaatcatatgccaatagaccgctgtctgttaaattcaaagaatcgttagcaaaattagtttcgccaattttataa

SEQ ID NO: 18 - protein, 493 aa, Staphylococcus aureus
MRFTFSNDLGTLFTIILAIGFIINLVLAFIIIFLERNRRTASSTWAWLFVLFVLPLIGFILYLFFGRTVSARKLNKNNGNVLTDFDGLLKQQIESFDKGNYGTDNKQVQKHHDLVRMLLMDQDGFLTENNKVDHFIDGNDLYDQVLKDIKNAKEYIHLEYYTFALDGLGKRILHALEEKLKQGLEVKILYDDVGSKNVKMANFDHFKSLGGEVEAFFASKLPLLNFRMNNRNHRKIIVIDGQLGYVGGFNIGDEYLGLGKLGYWRDTHLRIQGDAVDALQLRFILDWNSQAHRPQFEYDVKYFPKKNGPLGNSPIQIAASGPASDWHQIEYGYTKMIMSAKKSVYLQSPYFIPDNSYINAIKIAAKSGVDVHLMIPCKPDHPLVYWATFSNASDLLSSGVKIYTYENGFIHSKMCLIDDEIVSVGTANMDFRSFELNFEVNAFVYDENLAKDLRVAYEHDITKSKQLTKESYANRPLSVKFKESLAKLVSPIL

SEQ ID NO: 19 - DNA, 420 bp, Staphylococcus aureus
atgaagattttattcgtttgtacaggtaacacatgtcgtagcccattagcggaaagtattgcaaaagaggttatgccaaatcatcaatttgaatcaagaggtatattcgctgtgaacaatcaaggtgtttcgaattatgttgaagacttagttgaagaacatcatttagctgaaacgaccttatcgcaacaatttactgaagcagatttgaaagcagatattattttgacgatgtcgtattcgcacaaagaattaatagaggcacactttggtttgcaaaatcatgttttcacattgcatgaatatgtaaaagaagcaggagaagttatagatccatacggtggaacaaaagaaatgtatgtacatacctatgaagaacttgtaagtttaattttaaaattaaaagatattatttgctag

SEQ ID NO: 20 - protein, 139 aa, Staphylococcus aureus
MKILFVCTGNTCRSPLAESIAKEVMPNHQFESRGIFAVNNQGVSNYVEDLVEEHHLAETTLSQQFTEADLKADIILTMSYSHKELIEAHFGLQNHVFTLHEYVKEAGEVIDPYGGTKEMYVHTYEELVSLILKLKDIIC

SEQ ID NO: 21 - DNA, 837 bp, Staphylococcus aureus
atgacaaaacagattatagtaacagactcaacatccgatttatctaaagaatacttagaagcaaacaacattcatgtaattcctttaagtttaactattgaaggagcttcatacgttgaccaagtagatattacatcagaagaatttattaatcatattgaaaatgatgaagatgtaaagacaagtcagccagccataggtgaatttatatctgcttatgaagaactaggaaaagatggctctgaaatcataagtattcatctttcttcaggattaagtggtacatataacactgcttaccaagcaagtcaaatggtagatgctaatgtaactgttattgattcaaaatctatttcttttggtttagggtatcaaatacaacacctagtagagcttgtaaaagaaggtgtctcaacttctgaaatagttaaaaagttaaatcatttaagagaaaacattaaattatttgtagttatagggcaattgaatcaattaattaaaggtggcagaattagtaaaacaaaaggtttgattggtaatcttatgaaaattaaaccaattggtacactagatgatggtcgcttagagcttgtgcacaatgcgagaactcaaaattctagtatccaatacttgaaaaaggaaattgctgaatttataggagatcatgaaatcaaatccattggtgtcgcacatgctaacgtcattgaatatgttgataaattgaagaaagtttttaatgaagcttttcatgtgaataattacgatataaatgtaactacaccagttatttctgcacatactggtcaaggtgcgattggcctcgtagtccttaagaag

SEQ ID NO: 22 - protein, 279 aa, Staphylococcus aureus
MTKQIIVTDSTSDLSKEYLEANNIHVIPLSLTIEGASYVDQVDITSEEFINHIENDEDVKTSQPAIGEFISAYEELGKDGSEIISIHLSSGLSGTYNTAYQASQMVDANVTVIDSKSISFGLGYQIQHLVELVKEGVSTSEIVKKLNHLRENIKLFVVIGQLNQLIKGGRISKTKGLIGNLMKIKPIGTLDDGRLELVHNARTQNSSIQYLKKEIAEFIGDHEIKSIGVAHANVIEYVDKLKKVFNEAFHVNNYDINVTTPVISAHTGQGAIGLVVLKK

SEQ ID NO: 23 - DNA, 594 bp, Staphylococcus aureus
atgaatttattttacaatcctaaatatgtaggagatgtcgcatttttacaaattgaaccagttgaaggtgaattaaactacaataaaaaaggtaatgttgttgaaattactaatgaaggtaatgttgtaggttataatatttttgaaatttcaaaagatataacaattgaagaaaaaggtcatattaaattaactgatgaacttgtaaatgtattccaaaagcgtatttcagaagctggttttgattataaattaaatgctgatctatcaccgaaatttgtagttggctacgttgaaactaaagacaaacatcctgatgcagataaattaagtgtactaaatgtaaacgttggaaatgacacattacaaattgtatgtggcgcgcctaacgttgaagctggacagaaagttgttgttgctaaagtaggtgcagtgatgcctagcggtatggtaattaaagatgctgaattacgtggtgttgcctcaagcggtatgatttgttcaatgaaagaattgaatttacctaatgcacctgaagaaaaaggtattatggtattaaatgacagctatgaaattggacaagcattttttgaa

SEQ ID NO: 24 - protein, 198 aa, Staphylococcus aureus
MNLFYNPKYVGDVAFLQIEPVEGELNYNKKGNVVEITNEGNVVGYNIFEISKDITIEEKGHIKLTDELVNVFQKRISEAGFDYKLNADLSPKFVVGYVETKDKHPDADKLSVLNVNVGNDTLQIVCGAPNVEAGQKVVVAKVGAVMPSGMVIKDAELRGVASSGMICSMKELNLPNAPEEKGIMVLNDSYEIGQAFFE

SEQ ID NO: 25 - DNA, 717 bp, Staphylococcus aureus
atgactgtagaatggttagcagaacaattaaaagaacataatattcaattaactgagactcaaaaacaacagtttcaaacatattatcgtttacttgttgaatggaatgaaaagatgaatttgacaagtattacagatgaacacgatgtatatttgaaacatttttatgattccattgcacctagtttttattttgattttaatcagcctataagtatatgtgatgtaggcgctggagctggttttccaagtattccgttaaaaataatgtttccgcagttaaaagtgacgattgttgattcattaaataagcgtattcaatttttaaaccatttagcgtcagaattacaattacaggatgtcagctttatacacgatagagcagaaacatttggtaagggtgtctacagggagtcttatgatgttgttactgcaagagcagtagctagattatccgtgttaagtgaattgtgtttaccgctagttaaaaaaggtggacagtttgttgcattaaaatcttcaaaaggtgaagaagaattagaagaagcaaaatttgcaattagtgtgttaggtggtaatgttacagaaacacatacctttgaattgccagaagatgctggagagcgccagatgttcattattgataaaaaaagacagacgccgaaaaagtatccaagaaaaccagggacgcctaataagactcctttacttgaaaaa

SEQ ID NO: 26 - protein, 239 aa, Staphylococcus aureus
MTVEWLAEQLKEHNIQLTETQKQQFQTYYRLLVEWNEKMNLTSITDEHDVYLKHFYDSIAPSFYFDFNQPISICDVGAGAGFPSIPLKIMFPQLKVTIVDSLNKRIQFLNHLASELQLQDVSFIHDRAETFGKGVYRESYDVVTARAVARLSVLSELCLPLVKKGGQFVALKSSKGEEELEEAKFAISVLGGNVTETHTFELPEDAGERQMFIIDKKRQTPKKYPRKPGTPNKTPLLEK

SEQ ID NO: 27 - DNA, 2358 bp, Staphylococcus aureus
atgataatatattggtgtatgacagttaatggagggaacgaaatgaaagctttattacttaaaacaagtgtatggctcgttttgctttttagtgtaatgggattatggcaagtctcgaacgcggctgagcagcatacaccaatgaaagcacatgcagtaacaacgatagacaaagcaacaacagataagcaacaagtaccgccaacaaaggaagcggctcatcattctggcaaagaagcggcaaccaacgtatcagcatcagcgcagggaacagctgatgatacaaacagcaaagtaacatccaacgcaccatctaacaaaccatctacagtagtttcaacaaaagtaaacgaaacacgcgacgtagatacacaacaagcctcaacacaaaaaccaactcacacagcaacgttcaaattatcaaatgctaaaacagcatcactttcaccacgaatgtttgctgctaatgcaccacaaacaacaacacataaaatattacatacaaatgatatccatggccgactagccgaagaaaaagggcgtgtcatcggtatggctaaattaaaaacagtaaaagaacaagaaaagcctgatttaatgttagacgcaggagacgccttccaaggtttaccactttcaaaccagtctaaaggtgaagaaatggctaaagcaatgaatgcagtaggttatgatgctatggcagtcggtaaccatgaatttgactttggatacgatcagttgaaaaagttagagggtatgttagacttcccgatgctaagtactaacgtttataaagatggaaaacgcgcgtttaagccttcaacgattgtaacaaaaaatggtattcgttatggaattattggtgtaacgacaccagaaacaaagacgaaaacaagacctgaaggcattaaaggcgttgaatttagagatccattacaaagtgtgacagcggaaatgatgcgtatttataaagacgtagatacatttgttgttatatcacatttaggaattgatccttcaacacaagaaacatggcgtggtgattacttagtgaaacaattaagtcaaaatccacaattgaagaaacgtattacagttattgatggtcattcacatacagtacttcaaaatggtcaaatttataacaatgatgcattggcacaaacaggtacagcacttgcgaatatcggtaagattacatttaattatcgcaatggagaggtatcgaatattaaaccgtcattgattaatgttaaagacgttgaaaatgtaacaccgaacaaagcattagctgaacaaattaatcaagctgatcaaacatttagagcacaaactgcagaggtaattattccaaacaataccattgatttcaaaggagaaagagatgacgttagaacgcgtgaaacaaatttaggaaacgcgattgcagatgctatggaagcgtatggcgttaagaatttctctaaaaagactgactttgccgtgacaaatggtggaggtattcgtgcctctatcgcaaaaggtaaggtgacacgctatgatttaatctcagtattaccatttggaaatacgattgcgcaaattgatgtaaaaggttcagacgtctggacggctttcgaacatagtttaggcgcaccaacaacacaaaaggacggtaagacagtgttaacagcgaatggcggtttactacatatctctgattcaatccgtgtttactatgatataaataaaccgtctggcaaacgaattaatgctattcaaattttaaataaagagacaggtaagtttgaaaatattgatttaaaacgtgtatatcacgtaacgatgaatgacttcacagcatcaggtggcgacggatatagtatgttcggtggtcctagagaagaaggtatttcattagatcaagtactagcaagttatttaaaaacagctaacttagctaagtatgatacgacagaaccacaacgtatgttattaggtaaaccagcagtaagtgaacaaccagctaaaggacaacaaggtagcaaaggtagtaagtctggtaaagatacacaaccaattggtgacgacaaagtgatggatccagcgaaaaaaccagctccaggtaaagttgtattgttgctagcgcatagaggaactgttagtagcggtacagaaggttctggtcgcacaatagaaggagctactgtatcaagcaagagtgggaaacaattggctagaatgtcagtgcctaaaggtagcgcgcatgagaaacagttaccaaaaactggaactaatcaaagttcaagcccagaagcgatgtttgtattattagcaggtataggtttaatcgcgactgtacgacgtagaaaagctagc

SEQ ID NO: 28 - protein, 786 aa, Staphylococcus aureus
MIIYWCMTVNGGNEMKALLLKTSVWLVLLFSVMGLWQVSNAAEQHTPMKAHAVTTIDKATTDKQQVPPTKEAAHHSGKEAATNVSASAQGTADDTNSKVTSNAPSNKPSTVVSTKVNETRDVDTQQASTQKPTHTATFKLSNAKTASLSPRMFAANAPQTTTHKILHTNDIHGRLAEEKGRVIGMAKLKTVKEQEKPDLMLDAGDAFQGLPLSNQSKGEEMAKAMNAVGYDAMAVGNHEFDFGYDQLKKLEGMLDFPMLSTNVYKDGKRAFKPSTIVTKNGIRYGIIGVTTPETKTKTRPEGIKGVEFRDPLQSVTAEMMRIYKDVDTFVVISHLGIDPSTQETWRGDYLVKQLSQNPQLKKRITVIDGHSHTVLQNGQIYNNDALAQTGTALANIGKITFNYRNGEVSNIKPSLINVKDVENVTPNKALAEQINQADQTFRAQTAEVIIPNNTIDFKGERDDVRTRETNLGNAIADAMEAYGVKNFSKKTDFAVTNGGGIRASIAKGKVTRYDLISVLPFGNTIAQIDVKGSDVWTAFEHSLGAPTTQKDGKTVLTANGGLLHISDSIRVYYDINKPSGKRINAIQILNKETGKFENIDLKRVYHVTMNDFTASGGDGYSMFGGPREEGISLDQVLASYLKTANLAKYDTTEPQRMLLGKPAVSEQPAKGQQGSKGSKSGKDTQPIGDDKVMDPAKKPAPGKVVLLLAHRGTVSSGTEGSGRTIEGATVSSKSGKQLARMSVPKGSAHEKQLPKTGTNQSSSPEAMFVLLAGIGLIATVRRRKAS

SEQ ID NO: 29 - DNA, 609 bp, Staphylococcus aureus
atgagtcattattacgatgaagatccaagtgtaattagcaatgaacaacgtattcaatatcaattaaaccatcataaaattgatttaataactgataacggagtgttttcgaaagataaagtagattatggttcagatgttcttgttcaaacttttttaaaagcgcatccacctggtccaagtaagcgaattgccgatgttggttgtggttacggaccaattggtttgatgattgctaaagtatcaccacatcattcaattacaatgctagatgttaatcacagagcgctagccttagttgaaaaaaacaaaaaattaaatggtattgataatgtgatcgtaaaggaaagtgatgctttgtctgctgtggaagacaaaagttttgattttattttaaccaatccaccaataagagcagggaaagaaaccgtgcatcgtatattcgagcaagcattacatagattagactcgaacggtgaactattcgttgtaattcagaagaagcaaggtatgccatctgcaaagaaaagaatgaatgaactttttggaaatgtagaagtggtaaataaagataaaggatattacattctgagaagtataaaagcttga

SEQ ID NO: 30 - protein, 202 aa, Staphylococcus aureus
MSHYYDEDPSVISNEQRIQYQLNHHKIDLITDNGVFSKDKVDYGSDVLVQTFLKAHPPGPSKRIADVGCGYGPIGLMIAKVSPHHSITMLDVNHRALALVEKNKKLNGIDNVIVKESDALSAVEDKSFDFILTNPPIRAGKETVHRIFEQALHRLDSNGELFVVIRRKQGMPSAKKRMNELFGNVEVVNKDKGYYILRSIKA

SEQ ID NO: 31 - DNA, 750 bp, Staphylococcus aureus
atgcgagttgatttgaattgtgatttaggcgaagcatttggaaattattcctttggtggtgatcatcaaattattccgttaattacaagtgcgaatgttgcttgtggttttcacgctggtgatgaaaatgtaatgaatgaaacggtaaaacttgccaaagcacataatgttgcagtaggtgcacatcctggtttacctgatttgaaaggctttggcagacgaaatatagatatctctaacgacgagatttataatttgatgatttatcaattaggtgcattacaagggttttgtcgcattcatcaagttaaaattaatcatgttaaaccgcatggtgcattgtatcagatgggtgcaaaagacagagaaatagcaaacgttatagcacaagctgtttatgactttgatccatcactagtgttagtaggattagcaaattcatatctaatttcagaagcaaagaatgtcggattaattacagcttctgaagtgtttgctgatagacgatacgaagatgatgggcagctcgttagtagaaaagaaagtgatgctgtgattactgatactgacgaagcacttaagcaggttttaaagatggtgaaggaaaataaagttatttcaaaaaacaataaggaagtaacgttacaagcagatacaatttgtgtgcatggtgatggagaacatgcattattatttgtttcgaaaattagagaaattttaatgaaagaaggcattgatattcaatcctta

SEQ ID NO: 32 - protein, 250 aa, Staphylococcus aureus
MRVDLNCDLGEAFGNYSFGGDHQIIPLITSANVACGFHAGDENVMNETVKLAKAHNVAVGAHPGLPDLKGFGRRNIDISNDEIYNLMIYQLGALQGFCRIHQVKINHVKPHGALYQMGAKDREIANVIAQAVYDFDPSLVLVGLANSYLISEAKNVGLITASEVFADRRYEDDGQLVSRKESDAVITDTDEALKQVLKMVKENKVISKNNKEVTLQADTICVHGDGEHALLFVSKIREILMKEGIDIQSL

SEQ ID NO: 33 - DNA, 720 bp, Staphylococcus aureus
atggctcaaatttctaaatataaacgtgtagttttgaaactaagtggtgaagcgttagctggagaaaaaggatttggcataaatccagtaattattaaaagtgttgctgagcaagtggctgaagttgctaaaatggactgtgaaatcgcagtaatcgttggtggcggaaacatttggagaggtaaaacaggtagtgacttaggtatggaccgtggaactgctgattacatgggtatgcttgcaactgtaatgaatgccttagcattacaagatagtttagaacaattggattgtgatacacgagtattaacatctattgaaatgaagcaagtggctgaaccttatattcgtcgtcgtgcaattagacacttagaaaagaaacgcgtagttatttttgctgcaggtattggaaacccatacttctctacagatactacagcggcattacgtgctgcagaagttgaagcagatgttattttaatgggcaaaaataatgtagatggtgtatattctgcagatcctaaagtaaacaaagatgcggtaaaatatgaacatttaacgcatattcaaatgcttcaagaaggtttacaagtaatggattcaacagcatcctcattctgtatggataataacattccgttaactgttttctctattatggaagaaggaaatattaaacgtgctgttatgggtgaaaagataggtacgttaattacaaaa

SEQ ID NO: 34 - protein, 240 aa, Staphylococcus aureus
MAQISKYKRVVLKLSGEALAGEKGFGINPVIIKSVAEQVAEVAKMDCEIAVIVGGGNIWRGKTGSDLGMDRGTADYMGMLATVMNALALQDSLEQLDCDTRVLTSIEMKQVAEPYIRRRAIRHLEKKRVVIFAAGIGNPYFSTDTTAALRAAEVEADVILMGKNNVDGVYSADPKVNKDAVKYEHLTHIQMLQEGLQVMDSTASSFCMDNNIPLTVFSIMEEGNIKRAVMGEKIGTLITK

SEQ ID NO: 35 - DNA, 33 bp, Artificial
  Oligonucleotide Primer
atatatctgcagtgataaattgccaagcgtgac

SEQ ID NO: 36 - DNA, 33 bp, Artificial
  Oligonucleotide Primer
atatatgagctctcttgtacagatttaggtggc

SEQ ID NO: 37 - DNA, 33 bp, Artificial
  Oligonucleotide Primer
atatatctgcagcaagtattaggtgaagaaggg

SEQ ID NO: 38 - DNA, 33 bp, Artificial
  Oligonucleotide Primer
atatatgagctcacggattgatcccaataatgc

SEQ ID NO: 39 - DNA, 36 bp, Artificial
  Oligonucleotide Primer
atatatctgcaggggattcaaaaacaatttaacatc

SEQ ID NO: 40 - DNA, 36 bp, Artificial
  Oligonucleotide Primer
atatatgagctcaaggctattaacttgcttatgatc

SEQ ID NO: 41 - DNA, 21 bp, Artificial
  Oligonucleotide Primer
tggtgtgtacattgactgtac

SEQ ID NO: 42 - DNA, 21 bp, Artificial
  Oligonucleotide Primer
gctgttagttcttgtgtttgg

SEQ ID NO: 43 - DNA, 33 bp, Artificial
  Oligonucleotide Primer
atatatctgcagaggtattcaccatgttactgc

SEQ ID NO: 44 - DNA, 33 bp, Artificial
  Oligonucleotide Primer
atatatgagctcaattgatacacttggccatcg

SEQ ID NO: 45 - DNA, 35 bp, Artificial
  Oligonucleotide Primer
atatatctgcaggggacatttttaatcatgcatgc

SEQ ID NO: 46 - DNA, 36 bp, Artificial
  Oligonucleotide Primer
atatatgagctcgcagtcataataatagctaaagac

SEQ ID NO: 47 - DNA, 33 bp, Artificial
  Oligonucleotide Primer
atatatctgcagtgttaatcgatacacatgtcc

SEQ ID NO: 48 - DNA, 33 bp, Artificial
  Oligonucleotide Primer
atatatgagctccttcaaacgcttagctaaagc

SEQ ID NO: 49 - DNA, 33 bp, Artificial
  Oligonucleotide Primer
atatatctgcagacaagtgtatggctcgttttg

SEQ ID NO: 50 - DNA, 33 bp, Artificial
  Oligonucleotide Primer
atatatgagctcatttgaacgttgctgtgtgag

SEQ ID NO: 51 - DNA, 34 bp, Artificial
  Oligonucleotide Primer
atatatctgcagagagtacatactttcgctttag

SEQ ID NO: 52 - DNA, 34 bp, Artificial
  Oligonucleotide Primer
atatatgagctccctaatcctagatattcatcac

SEQ ID NO: 53 - DNA, 33 bp, Artificial
  Oligonucleotide Primer
atatatctgcagttgtacaggtaacacatgtcg

SEQ ID NO: 54 - DNA, 32 bp, Artificial
  Oligonucleotide Primer
atatatgagctcctgctttcaaatctgctcag

SEQ ID NO: 55 - DNA, 21 bp, Artificial
  Oligonucleotide Primer
ctattgaaggagcttcatacg

SEQ ID NO: 56 - DNA, 21 bp, Artificial
  Oligonucleotide Primer
tacaagctctactaggtgttg

SEQ ID NO: 57 - DNA, 21 bp, Artificial
  Oligonucleotide Primer
cctaaatatgtaggagatgtc

SEQ ID NO: 58 - DNA, 21 bp, Artificial
  Oligonucleotide Primer
ttatctgcatcaggatgtttg

SEQ ID NO: 59 - DNA, 21 bp, Artificial
  Oligonucleotide Primer
ctgtagaatggttagcagaac

SEQ ID NO: 60 - DNA, 21 bp, Artificial
  Oligonucleotide Primer
caatcgtcacttttaactgcg

SEQ ID NO: 61 - DNA, 21 bp, Artificial
  Oligonucleotide Primer
acaagtgtatggctcgttttg

SEQ ID NO: 62 - DNA, 21 bp, Artificial
  Oligonucleotide Primer
atttgaacgttgctgtgtgag

SEQ ID NO: 63 - DNA, 21 bp, Artificial
  Oligonucleotide Primer
attacgatgaagatccaagtg

SEQ ID NO: 64 - DNA, 21 bp, Artificial
  Oligonucleotide Primer
ttttttcaactaaggctagcg

SEQ ID NO: 65 - DNA, 21 bp, Artificial
  Oligonucleotide Primer
gaattgtgatttaggcgaagc

SEQ ID NO: 66 - DNA, 21 bp, Artificial
  Oligonucleotide Primer
atgaatgcgacaaaacccttg

SEQ ID NO: 67 - DNA, 21 bp, Artificial
  Oligonucleotide Primer
ttgaaactaagtggtgaagcg

SEQ ID NO: 68 - DNA, 21 bp, Artificial
  Oligonucleotide Primer
gcagcaaaaataactacgcgt

SEQ ID NO: 69 - DNA, 731 bp, Artificial
  Nucleotide sequence of S. aureus coding region cloned for express ion in E. col
atgggattaaagtatgaacatattgctaagcaacttaatgcgtttatacatcaatctaatttcaaacccggtgataaattgccaagcgtgacgcaattaaaagaacgttatcaagtaagtaagagtactatcattaaagcattaggcttattggaacaagatggtttgatctatcaagcacaaggcagtggtatttatgtgagaaatattgctgatgccaatcgtatcaacgtctttaagactaatggtttctctaaaagtttaggtgaacaccgaatgacaagtaaggtacttgtttttaaggagattgcaacgccacctaaatctgtacaagatgagctccaattaaatgcagatgataccgtctactatttagagcgattaagattcgtggacgatgatgttttatgtatcgaatattcttattatcataaagaaatcgtgaaatatttaaatgatgatattgctaagggctctatcttcgacttttagaatcaaacatgaaacttcgtattggtttttcagatattttctttaatgtagatcaactcacttcaagtgaagcttcattactacaattgtctacaggtgaaccatgtttacgttaccaccagactttttatacaatgactggcaaaccctttgattcatctgacatcgtatttcattatcgtcatgcacagttttatattcctagtaaaaagagatctcatcaccatcaccatcactaa

SEQ ID NO: 70 - protein, 243 aa, Artificial
  Amino acid sequence encoded by S. aureus coding region cloned for expression in E. col
MGLKYEHIAKQLNAFIHQSNFKPGDKLPSVTQLKERYQVSKSTIIKALGLLEQDGLIYQAQGSGIYVRNIADANRINVFKTNGFSKSLGEHRMTSKVLVFKEIATPPKSVQDELQLNADDTVYYLERLRFVDDDVLCIEYSYYHKEIVKYLNDDIAKGSIFDYLESNMKLRIGFSDIFFNVDQLTSSEASLLQLSTGEPCLRYHQTFYTMTGKPFDSSDIVFHYRHAQFYIPSKKRSHHHHHH

SEQ ID NO: 71 - DNA, 855 bp, Artificial
  Nucleotide sequence of S. aureus coding region cloned for express ion in E. col
atgctggcactttatggatttgcccaaggacttattcaagaagcaggaattagaattaaacaattgatggagcaaaatttaacaattgaaacaaagtcaaatccgaatgaccttgttacaaatgtagataaagcaacagaagatttcatttttgatacaattttagaaacatatcccaatcatcaagtattaggtgaagaagggcatggtcatgacatcgatacttccaaaggtacggtatggattgttgacccaatagacggtacattgaattttgttcatcaacaagaaaatttcgcaatttcaattggtatttatatcgatggtaaaccttatgcaggttttgtatatgatgttatggctgatgtcttatatcatgctaaagtaggggaaggtgcatatcgtggtagccaacccttgaaaccattgaatgattctaatctaagacaaagcattattgggatcaatccgaactggttaactaaaccaattttaggagaaatctttaaagaaattgttaatgattctagaagtgcaagggcatatggtagtgcagcgcttgaaatcgtttcagttgctacaggtaatttagaagcatatatgacgccaagacttcaaccatgggattttgctggcggattggttattttatatgaagtaaatggacaagcttccaatttactaggaggaccattaacaattagtggtccaaattcaatcttagttggaaatcgtggtctccatcaagaaattagcaatgattatttagagccccaccatgatgcgttaatacaattacatgaacaacgatttaaaagaaaatcaaaaagatctcatcaccatcaccatcactaa

SEQ ID NO: 72 - protein, 284 aa, Artificial
  Amino acid sequence encoded by S. aureus coding region cloned for expression in E. col
MLALYGFAQGLIQEAGIRIKQLMEQNLTIETKSNPNDLVTNVDKATEDFIFDTILETYPNHQVLGEEGHGHDIDTSKGTVWIVDPIDGTLNFVHQQENFAISIGIYIDGKPYAGFVYDVMADVLYHAKVGEGAYRGSQPLKPLNDSNLRQSIIGINPNWLTKPILGEIFKEIVNDSRSARAYGSAALEIVSVATGNLEAYMTPRLQPWDFAGGLVILYEVNGQASNLLGGPLTISGPNSILVGNRGLHQEISNDYLEPHHDALIQLHEQRFKRKSKRSHHHHHH

SEQ ID NO: 73 - DNA, 567 bp, Artificial
  Nucleotide sequence of S. aureus coding region cloned for express ion in E. col
atgggattcaaaaacaatttaacatcaaatttaacaaataaaatcggtaattcagtctttaaaatagaaaatgttgacggaaaaggtgcaatgccaacgacgattcaagaattgagagaaagacgacaacgtgctgaagcaattgtaaagagaaagtctttaatgtcatcaacaatgagcgttgttccaattccgggtttagattttggtgttgatttaaaattaatgaaagatattatcgaagatgttaataaaatttatggtttagatcataagcaagttaatagccttggggatgatgtgaaagaaagaattatgtctgcagcagcaattcaaggtagtcaatttattggtaaaagaatttcaaatgcatttttaaaaattgtaattagagatgtagctaaacgtactgctgcaaaacaaacaaaatggtttcctgttgtaggacaagctgtgtctgcatctattagttactattttatgaataaaattggaaaagatcacattcaaaaatgcgaaaatgttattaaaaatgtcatgagatctcatcaccatcaccatcactaa

SEQ ID NO: 74 - protein, 188 aa, Artificial
  Amino acid sequence encoded by S. aureus coding region cloned for expression in E. col
MGFKNNLTSNLTNKIGNSVFKIENVDGKGAMPTTIQELRERRQRAEAIVKRKSLMSSTMSVVPIPGLDFGVDLKLMKDIIEDVNKIYGLDHKQVNSLGDDVKERIMSAAAIQGSQFIGKRISNAFLKIVIRDVAKRTAAKQTKWFPVVGQAVSASISYYFMNKIGKDHIQKCENVIKNVMRSHHHHHH

SEQ ID NO: 75 - DNA, 996 bp, Artificial
  Nucleotide sequence of S. aureus coding region cloned for express ion in E. col
atgggaataaataatcatgaattactaggtattcaccatgttactgcaatgacagatgatgcagaacgtaattataaattttttacagaagtactaggcatgcgtttagttaaaaagacagtcaatcaagatgatatttatacgtatcatactttttttgcagatgatgtaggttcggcaggtacagacatgacgttctttgattttccaaatattacaaaagggcaggcaggaacaaattccattacaagaccgtcttttagagtgcctaacgatgacgcattaacatattatgaacagcgctttgatgagtttggtgttaaacacgaaggtattcaagaattatttggtaaaaaagtgttgccatttgaagaagtcgatggccaagtgtatcaattaatttcagatgagttaaatgaaggggtagcacctggtgtaccttggaagaatggaccggttccagtagataaagcgatttatggattaggccccattgaaattaaagtaagttattttgacgactttaaaaatattttagagactgtttacggtatgacaactattgcgcatgaagataatgtcgcattacttgaagttggcgaaggaggcaatggtggccaggtaatcttaataaaagatgataaagggccagcagcacgtcaaggttatggtgaggtacatcatgtgtcatttcgtgtgaaagatcatgatgcaatagaagcgtgggcaacgaaatataaagaggtaggtattaataactcaggcatcgttaatcgtttctattttgaagcattatatgcacgtgtggggcatattttaatagaaatttcaacagatggaccaggatttatggaagatgaaccttatgaaacattaggcgaagggttatccttaccaccatttttagaaaataaaagagaatatattgaatcggaagttagaccttttaatacgaagcgtcaacatggtagatctcatcaccatcaccatcactaa

SEQ ID NO: 76 - protein, 331 aa, Artificial
  Amino acid sequence encoded by S. aureus coding region cloned for expression in E. col
MGINNHELLGIHHVTAMTDDAERNYKFFTEVLGMRLVKKTVNQDDIYTYHTFFADDVGSAGTDMTFFDFPNITKGQAGTNSITRPSFRVPNDDALTYYEQRFDEFGVKHEGIQELFGKKVLPFEEVDGQVYQLISDELNEGVAPGVPWKNGPVPVDKAIYGLGPIEIKVSYFDDFKNILETVYGMTTIAHEDNVALLEVGEGGNGGQVILIKDDKGPAARQGYGEVHHVSFRVKDHDAIEAWATKYKEVGINNSGIVNRFYFEALYARVGHILIEISTDGPGFMEDEPYETLGEGLSLPPFLENKREYIESEVRPFNTKRQHGRSHHHHHH

SEQ ID NO: 77 - DNA, 1113 bp, Artificial
  Nucleotide sequence of S. aureus coding region cloned for express ion in E. col
atgggacgtatcttaaaagagtccattattgtggcatttgcctttgttggtgttgtcgttggtgccggctttgctactggtcaagaaattttccagtttttcacaagtcatggcgcatatagcatttcaggcattattgtaacaggactattgattactttaggtggaatggttgtcatgcatacaggtcatcatctaaagtccagaaatcattctgattcaattaactatttcttatacccctctattgcaagaggttttgatattattttaacaatgtttatgttgtctttagctattattatgactgcaggtggtgcgtcaaccattcatcaaagtttcaacttaccgtattggctgagcgcactcatattagtcgcctttattttagcaacactgtttctaaaattcgatcgtttaattgctgtgcttggcggtgttaccccatttttaattgcgattgtcattatgattgcggtctactatttcacaacaagtcatcttgattttactgccgctaataatgatgctcagattcataagcagaaatcattatcacctggatggtggtttgatgcgattaactatgcaagcttgcaaattgctgctgccttcagcttcttatcagtgatgggtagtaaagttaaatatcgtgactcaacgttatacgggggcttgattggcggtttaatcattacatttttactcatgatgattaatctaggtttaatttctcaattcgataaaattaaacacgtagatctacctacattaaaattagcgacacaaatgtctccgtcaattggtattattatgtctgtcattatgatacttgtcatctacaatactgttgttggattaatgtatgcatttgcgtcacgtttcagcgttccattcagcagacgttacttcatcattattattacaatggctgtcatcacttatattagtacatttatcggtttcatttcattaattggaaaagtattccctattatgggattgttcggtttcatcttactcatacctgtactctataaaggtttaattaagcgtattaccggcaaatctcatatcgatggatccagatctcatcaccatcaccatcactaa

SEQ ID NO: 78 - protein, 370 aa, Artificial
  Amino acid sequence encoded by S. aureus coding region cloned for expression in E. col
MGRILKESIIVAFAFVGVVVGAGFATGQEIFQFFTSHGAYSISGIIVTGLLITLGGMVVMHTGHHLKSRNHSDSINYFLYPSIARGFDIILTMFMLSLAIIMTAGGASTIHQSFNLPYWLSALILVAFILATLFLKFDRLIAVLGGVTPFLIAIVIMIAVYYFTTSHLDFTAANNDAQIHKQKSLSPGWWFDAINYASLQIAAAFSFLSVMGSKVKYRDSTLYGGLIGGLIITFLLMMINLGLISQFDKIKHVDLPTLKLATQMSPSIGIIMSVIMILVIYNTVVGLMYAFASRFSVPFSRRYFIIIITMAVITYISTFIGFISLIGKVFPIMGLFGFILLIPVLYKGLIKRITGKSHIDGFRSHHHHHH

SEQ ID NO: 79 - DNA, 801 bp, Artificial
  Nucleotide sequence of S. aureus coding region cloned for expression in E. col
atgggattaatcgatacacatgtccatttaaatgatgagcaatacgatgatgatttgagtgaagtgattacacgtgctagagaagcaggtgttgatcgtatgtttgtagttggttttaacaaatcgacaattgaacgcgcgatgaaattaatcgatgagtatgattttttatatggcattatcggttggcatccagttgacgcaattgattttacagaagaacacttggaatggattgaatctttagctcagcatccaaaagtgattggtattggtgaaatgggattagattatcactgggataaatctcctgcagatgttcaaaaggaagtttttagaaagcaaattgctttagctaagcgtttgaagttaccaattatcattcataaccgtgaagcaactcaagactgtatcgatatcttattggaggagcatgctgaagaggtaggcgggattatgcatagctttagtggttctccagaaattgcagatattgtaactaataagctgaatttttatatttcattaggtggacctgtgacatttaaaaatgctaaacagcctaaagaagttgctaagcatgtgtcaatggagcgtttgctagttgaaaccgatgcaccgtatctttcgccacatccgtatagagggaagcgaaatgaaccggcgagagtaactttagtagctgaacaaattgctgaattaaaaggcttatcttatgaagaagtgtgcgaacaaacaactaaaaatgcagagaaattgtttaatttaaattcaagatctcatcaccatcaccatcactaa

SEQ ID NO: 80 - protein, 266 aa, Artificial
  Amino acid sequence encoded by S. aureus coding region cloned for expression in E. col
MGLIDTHVHLNDEQYDDDLSEVITRAREAGVDRMFVVGFNKSTIERAMKLIDEYDFLYGIIGWHPVDAIDFTEEHLEWIESLAQHPKVIGIGEMGLDYHWDKSPADVQKEVFRKQIALAKRLKLPIIIHNREATQDCIDILLEEHAEEVGGIMHSFSGSPEIADIVTNKLNFYISLGGPVTFKNAKQPKEVAKHVSMERLLVETDAPYLSPHPYRGKRNEPARVTLVAEQIAELKGLSYEEVCEQTTKNAEKLFNLNSRSHHHHHH

SEQ ID NO: 81 - DNA, 2150 bp, Artificial
  Nucleotide sequence of S. aureus coding region cloned for expression in E. col
atgggaataatatattggtgtatgacagttaatggagggaacgaaatgaaagctttattacttaaaacaagtgtatggctcgttttgctttttagtgtaatgggattatggcaagtctcgaacgcggctgagcagcatacaccaatgaaagcacatgcagtaacaacgatagacaaagcaacaacagataagcaacaagtaccgccaacaaaggaagcggctcatcattctggcaaagaagcggcaaccaacgtatcagcatcagcgcagggaacagctgatgatacaaacagcaaagtaacatccaacgcaccatctaacaaaccatctacagtagtttcaacaaaagtaaacgaaacacgcgacgtagatacacaacaagcctcaacacaaaaaccaactcacacagcaacgttcaaattatcaaatgctaaaacagcatcactttcaccacgaatgtttgctgctaatgcaccacaaacaacaacacataaaatattacatacaaatgatatccatggccgactagccgaagaaaaagggcgtgtcatcggtatggctaaattaaaaacagtaaaagaacaagaaaagcctgatttaatgttagacgcaggagacgccttccaaggtttaccactttcaaaccagtctaaaggtgaagaaatggctaaagcaatgaatgcagtaggttatgatgctatggcagtcggtaaccatgaatttgactttggatacgatcagttgaaaaagttagagggtatgttagacttcccgatgctaagtactaacgtttataaagatggaaaacgcgcgtttaagccttcaacgattgtaacaaaaaatggtattcgttatggaattattggtgtaacgacaccagaaacaaagacgaaaacaagacctgaaggcattaaaggcgttgaatttagagatccattacaaagtgtgacagcggaaatgatgcgtatttataaagacgtagatacatttgttgttatatcacatttaggaattgatccttcaacacaagaaacatggcgtggtgattacttagtgaaacaattaagtcaaaatccacaattgaagaaacgtattacagttattgatggtcattcacatacagtacttcaaaatggtcaaatttataacaatgatgcattggcacaaacaggtacagcacttgcgaatatcggtaagattacatttaattatcgcaatggagaggtatcgaatattaaaccgtcattgattaatgttaaagacgttgaaaatgtaacaccgaacaaagcattagctgaacaaattaatcaagctgatcaaacatttagagcacaaactgcagaggtaattattccaaacaataccattgatttcaaaggagaaagagatgacgttagaacgcgtgaaacaaatttaggaaacgcgattgcagatgctatggaagcgtatggcgttaagaatttctctaaaaagactgactttgccgtgacaaatggtggaggtattcgtgcctctatcgcaaaaggtaaggtgacacgctatgatttaatctcagtattaccatttggaaatacgattgcgcaaattgatgtaaaaggttcagacgtctggacggctttcgaacatagtttaggcgcaccaacaacacaaaaggacggtaagacagtgttaacagcgaatggcggtttactacatatctctgattcaatccgtgtttactatgatataaataaaccgtctggcaaacgaattaatgctattcaaattttaaataaagagacaggtaagtttgaaaatattgatttaaaacgtgtatatcacgtaacgatgaatgacttcacagcatcaggtgggacggatatagtatgttcggtggtcctagagaagaaggtatttcattagatcaagtactagcaagttatttaaaaacagctaacttagctaagtatgatacgacagaaccacaacgtatgttattaggtaaaccagcagtaagtgaacaaccagctaaaggacaacaaggtagcaaaggtagtaagtctggtaaagatacacaaccaattggtgacgacaaagtgatggatccagcgaaaaaaccagctccaggtaaagttgtattgttgagatctcatcaccatcaccatcactaa

SEQ ID NO: 82 - protein, 716 aa, Artificial
  Amino acid sequence encoded by S. aureus coding region cloned expression in E. col
MGIIYWCMTVNGGNEMKALLLKTSVWLVLLFSVMGLWQVSNAAEQHTPMKAHAVTTIDKATTDKQQVPPTKEAAHHSGKEAATNVSASAQGTADDTNSKVTSNAPSNKPSTVVSTKVNETRDVDTQQASTQKPTHTATFKLSNAKTASLSPRMFAANAPQTTTHKILHTNDIHGRLAEEKGRVIGMAKLKTVKEQEKPDLMLDAGDAFQGLPLSNQSKGEEMAKAMNAVGYDAMAVGNHEFDFGYDQLKKLEGMLDFPMLSTNVYKDGKRAFKPSTIVTKNGIRYGIIGVTTPETKTKTRPEGIKGVEFRDPLQSVTAEMMRIYKDVDTFVVISHLGIDPSTQETWRGDYLVKQLSQNPQLKKRITVIDGHSHTVLQNGQIYNNDALAQTGTALANIGKITFNYRNGEVSNIKPSLINVKDVENVTPNKALAEQINQADQTFRAQTAEVIIPNNTIDFKGERDDVRTRETNLGNAIADAMEAYGVKNFSKKTDFAVTNGGGIRASIAKGKVTRYDLISVLPFGNTIAQIDVKGSDVWTAFEHSLGAPTTQKDGKTVLTANGGLLHISDSIRVYYDINKPSGKRINAIQILNKETGKFENIDLKRVYHVTMNDFTASGGDGYSMFGGPREEGISLDQVLASYLKTANLAKYDTTEPQRMLLGKPAVSEQPAKGQQGSKGSKSGKDTQPIGDDKVMDPAKKPAPGKVVLLRSHHHHHH

SEQ ID NO: 83 - DNA, 1509 bp, Artificial
  Nucleotide sequence of S. aureus coding region cloned for expression in E. col
atgggacgatttacattttcaaacgatttaggaacgttatttactattattttagccattggattcatcattaatttagtattggcttttattattatctttttagaaagaaataggcgtacagcgagttcaacttgggcatggctatttgtactttttgtcttaccattgattggttttattctttacttgttttttggtagaaccgtttcggcacgcaaattgaataaaaacaatggtaacgtgttaacggatttcgatggacttttaaaacaacaaatagaaagctttgataaaggtaattatggtactgataacaaacaagttcaaaaacatcatgatttagtacgtatgcttttgatggatcaagatggttttttaactgaaaataataaagttgatcatttcattgatggaaatgatttatatgatcaagttttaaaagatattaaaaatgcaaaagaatatatccatttagagtactatactttcgctttagatggtttaggtaaaagaattttacatgctttagaagaaaaattgaaacaaggtctagaagtaaaaatattatatgatgatgttggatctaaaaatgttaagatggcaaattttgatcattttaaatcgttaggtggagaagttgaagcattttttgcttcaaaattaccgttattgaatttcagaatgaataatagaaatcatagaaaaatcatcgtaatcgatggtcaactaggttatgtcggaggatttaacattggtgatgaatatctaggattaggaaaattaggatattggagagatacgcatttacgtatacaaggggatgcggttgatgcactgcagttgcgatttattttagactggaattcgcaagcgcaccgtccacaatttgaatatgatgttaagtatttccctaaaaagaacggaccattgggcaattcaccaattcaaatagctgcaagtggcccggctagtgactggcatcaaattgaatacggttatacaaaaatgattatgagtgcaaagaaatctgtatatttacaatcaccatatttcattccggataattcatatataaatgccattaaaattgctgctaaatcaggtgtagatgtacatttaatgattccatgtaagccagatcatccattagtatattgggcgacattttcaaatgcctctgacttattatcaagtggtgttaaaatttatacgtatgaaaatggatttatacattctaaaatgtgcttaattgatgatgaaatcgtatcagtgggcacagcaaatatggactttagaagttttgaattaaattttgaagtaaatgcctttgtatatgatgaaaatcttgctaaagatttaagggtggcttatgaacatgatattacaaaatcaaaacaactaaccaaagaatcatatgccaatagaccgctgtctgttaaattcaaagaatcgttagcaaaattagtttcgccaattttaagatctcatcaccatcaccatcactaa

SEQ ID NO: 84 - protein, 502 aa, Artificial
  Amino acid sequence encoded by S. aureus coding region cloned for expression in E. col
MGRFTFSNDLGTLFTIILAIGFIINLVLAFIIIFLERNRRTASSTWAWLFVLFVLPLIGFILYLFFGRTVSARKLNKNNGNVLTDFDGLLKQQIESFDKGNYGTDNKQVQKHHDLVRMLLMDQDGFLTENNKVDHFIDGNDLYDQVLKDIKNAKEYIHLEYYTFALDGLGKRILHALEEKLKQGLEVKILYDDVGSKNVKMANFDHFKSLGGEVEAFFASKLPLLNFRMNNRNHRKIIVIDGQLGYVGGFNIGDEYLGLGKLGYWRDTHLRIQGDAVDALQLRFILDWNSQAHRPQFEYDVKYFPKKNGPLGNSPIQIAASGPASDWHQIEYGYTKMIMSAKKSVYLQSPYFIPDNSYINAIKIAAKSGVDVHLMIPCKPDHPLVYWATFSNASDLLSSGVKIYTYENGFIHSKMCLIDDEIVSVGTANMDFRSFELNFEVNAFVYDENLAKDLRVAYEHDITKSKQLTKESYANRPLSVKFKESLAKLVSPILRSHHHHHH

SEQ ID NO: 85 - DNA, 447 bp, Artificial
  Nucleotide sequence of S. aureus coding region cloned for expression in E. col
atgggaaagattttattcgtttgtacaggtaacacatgtcgtagcccattagcggaaagtattgcaaaagaggttatgccaaatcatcaatttgaatcaagaggtatattcgctgtgaacaatcaaggtgtttcgaattatgttgaagacttagttgaagaacatcatttagctgaaacgaccttatcgcaacaatttactgaagcagatttgaaagcagatattattttgacgatgtcgtattcgcacaaagaattaatagaggcacactttggtttgcaaaatcatgttttcacattgcatgaatatgtaaaagaagcaggagaagttatagatccatacggtggaacaaaagaaatgtatgtacatacctatgaagaacttgtaagtttaattttaaaattaaaagatattatttgcagatctcatcaccatcaccatcactaa

SEQ ID NO: 86 - protein, 148 aa, Artificial
  Amino acid sequence encoded by S. aureus coding region cloned for expression in E. col
MGKILFVCTGNTCRSPLAESIAKEVMPNHQFESRGIFAVNNQGVSNYVEDLVEEHHLAETTLSQQFTEADLKADIILTMSYSHKELIEAHFGLQNHVFTLHEYVKEAGEVIDPYGGTKEMYVHTYEELVSLILKLKDIICRSHHHHHH

SEQ ID NO: 87 - DNA, 744 bp, Staphylococcus aureus
atggctcaaatttctaaatataaacgtgtagttttgaaactaagtggtgaagcgttagctggagaaaaaggatttggcataaatccagtaattattaaaagtgttgctgagcaagtggctgaagttgctaaaatggactgtgaaatcgcagtaatcgttggtggcggaaacatttggagaggtaaaacaggtagtgacttaggtatggaccgtggaactgctgattacatgggtatgcttgcaactgtaatgaatgccttagcattacaagatagtttagaacaattggattgtgatacacgagtattaacatctattgaaatgaagcaagtggctgaaccttatattcgtcgtcgtgcaattagacacttagaaaagaaacgcgtagttatttttgctgcaggtattggaaacccatacttctctacagatactacagcggcattacgtgctgcagaagttgaagcagatgttattttaatgggcaaaaataatgtagatggtgtatattctgcagatcctaaagtaaacaaagatgcggtaaaatatgaacatttaacgcatattcaaatgcttcaagaaggtttacaagtaatggattcaacagcatcctcattctgtatggataataacattccgttaactgttttctctattatggaagaaggaaatattaaacgtgctgttatgggtgaaaagataggtacgttaattacaaaaagatctcatcaccatcaccatcac

SEQ ID NO: 88 - protein, 248 aa, Staphylococcus aureus
MAQISKYKRVVLKLSGEALAGEKGFGINPVIIKSVAEQVAEVAKMDCEIAVIVGGGNIWRGKTGSDLGMDRGTADYMGMLATVMNALALQDSLEQLDCDTRVLTSIEMKQVAEPYIRRRAIRHLEKKRVVIFAAGIGNPYFSTDTTAALRAAEVEADVILMGKNNVDGVYSADPKVNKDAVKYEHLTHIQMLQEGLQVMDSTASSFCMDNNIPLTVFSIMEEGNIKRAVMGEKIGTLITKRSHHHHHH

SEQ ID NO: 89 - DNA, 162 bp, Artificial
  DNA sequence of portion of pQE-60 vector
ctcgagaaatcataaaaaatttatttgctttgtgagcggataacaattataatagattcaattgtgagcggataacaatttcacacagaattcattaaagaggagaaattaaccatgggaggatccagatctcatcaccatcaccatcactaagcttaatta

SEQ ID NO: 90 - DNA, 20 bp, Artificial
  Oligonucleotide Primer
cccgggccatggctcaaatt

SEQ ID NO: 91 - DNA, 33 bp, Artificial
  Oligonucleotide Primer
ccatgggattaaagtatgaacatattgctaagc

SEQ ID NO: 92 - DNA, 40 bp, Artificial
  Oligonucleotide Primer
gagatctctttttactaggaatataaaactgtgcatgacg

SEQ ID NO: 93 - DNA, 31 bp, Artificial
  Oligonucleotide Primer
gcatgctggcactttatggatttgcccaagg

SEQ ID NO: 94 - DNA, 40 bp, Artificial
  Oligonucleotide Primer
gagatctttttgattttcttttaaatcgttgttcatgatt

SEQ ID NO: 95 - DNA, 28 bp, Artificial
  Oligonucleotide Primer
ccatgggattcaaaaacaatttaacatc

SEQ ID NO: 96 - DNA, 33 bp, Artificial
  Oligonucleotide Primer
gagatctcatgacatttttaataacattttcgc

SEQ ID NO: 97 - DNA, 31 bp, Artificial
  Oligonucleotide Primer
ccatgggaataaataatcatgaattactagg

SEQ ID NO: 98 - DNA, 36 bp, Artificial
  Oligonucleotide Primer
gagatctaccatgttgacgcttcgtattaaaaggtc

SEQ ID NO: 99 - DNA, 36 bp, Artificial
  Oligonucleotide Primer
ccatgggacgtatcttaaaagagtccattattgtgg

SEQ ID NO: 100 - DNA, 34 bp, Artificial
  Oligonucleotide Primer
ggatccatcgatatgagatttgccggtaatacgc

SEQ ID NO: 101 - DNA, 29 bp, Artificial
  Oligonucleotide Primer
ccatgggattaatcgatacacatgtccat

SEQ ID NO: 102 - DNA, 45 bp, Artificial
  Oligonucleotide Primer
gagatcttgaatttaaattaaacaatttctctgcatttttagttg

SEQ ID NO: 103 - DNA, 30 bp, Artificial
  Oligonucleotide Primer
ccatgggaataatatattggtgtatgacag

SEQ ID NO: 104 - DNA, 34 bp, Artificial
  Oligonucleotide Primer
gagatctcaacaatacaactttacctggagctgg

SEQ ID NO: 105 - DNA, 34 bp, Artificial
  Oligonucleotide Primer
ccatgggacgatttacattttcaaacgatttagg

SEQ ID NO: 106 - DNA, 36 bp, Artificial
  Oligonucleotide Primer
gagatcttaaaattggcgaaactaattttgctaacg

SEQ ID NO: 107 - DNA, 35 bp, Artificial
  Oligonucleotide Primer
ccatgggaaagattttattcgtttgtacaggtaac

SEQ ID NO: 108 - DNA, 43 bp, Artificial
  Oligonucleotide Primer
gagatctgcaaataatatcttttaattttaaaattaaagaatg

SEQ ID NO: 109 - DNA, 3198 bp, Staphylococcus aureus
atggtggcatatttaaatattcatacggcttatgatttgttaaattcaagcttaaaaatagaagatgccgtaagacttgctgtgtctgaaaatgttgatgcacttgccataactgacaccaatgtattgtatggttttcctaaattttatgatgcatgtatagcaaataacattaaaccgatttttggtatgacaatatatgtgacaaatggattaaatacagtcgaaacagttgttctagctaaaaataatgatggattaaaagatttgtatcaactatcatcggaaataaaaatgaatgcattagaacatgtgtcgtttgaattattaaaacgattttctaacaatatgattatcatttttaaaaaagtcggtgatcaacatcgtgatattgtacaagtgtttgaaacccataatgacacatatatggaccaccttagtatttcgattcaaggtagaaaacatgtttggattcaaaatgtttgttaccaaacacgtcaagatgccgatacgatttctgcattagcagctattagagacaatacaaaattagacttaattcatgatcaagaagattttggtgcacattttttaactgaaaaggaaattaatcaattagatattaaccaagaatatttaacgcaggttgatgttatagctcaaaagtgtgatgcagaattaaaatatcatcaatctctacttcctcaatatgagacacctaatgatgaatcagctaaaaaatatttgtggcgtgtcttagttacacaattgaaaaaattagaacttaattatgacgtctatttagagcgattgaaatatgagtataaagttattactaatatgggttttgaagattatttcttaatagtaagtgatttaatccattatgcgaaaacgaatgatgtgatggtaggtcctggtcgtggttcttcagctggctcactggtcagttatttattgggaattacaacgattgatcctattaaattcaatctattatttgaacgttttttaaacccagaacgtgtaacaatgcctgatattgatattgactttgaagatacacgccgagaaagggtcattcagtacgtccaagaaaaatatggcgagctacatgtatctggaattgtgactttcggtcatctgcttgcaagagcagttgctagagatgttggaagaattatggggtttgatgaagttacattaaatgaaatttcaagtttaatcccacataaattaggaattacacttgatgaagcatatcaaattgacgattttaaagagtttgtacatcgaaaccatcgacatgaacgctggttcagtatttgtaaaaagttagaaggtttaccaagacatacatctacacatgcggcaggaattattattaatgaccatccattatatgaatatgcccctttaacgaaaggggatacaggattattaacgcaatggacaatgactgaagccgaacgtattgggttattaaaaatagattttctagggttgagaaacttatcgattattcatcaaatcttaacacaagtcaaaaaagatttaggtattaatattgatatcgaaaagattccgtttgatgatcaaaaagtgtttgaattgttgtcgcaaggagatacgactggcatattccaattagagtctgacggtgtaagaagtgtattaaaaaaattaaagccggaacactttgaagatattgttgctgtaacttctttgtatagaccaggtccaatggaagaaattccaacttacattacaagaagacatgatccaagcaaagttcaatatttacatccgcatttagaacctatattaaaaaatacttacggtgttattatttatcaagagcaaattatgcaaatagcgagcacatttgcaaacttcagttatggtgaagcggatattttaagaagagcaatgagtaaaaaaaatagagctgttcttgaaagtgagcgtcaacattttatagaaggtgcaaagcaaaatggttatcacgaagacattagtaagcaaatatttgatttgattctgaaatttgctgattatggttttcctagagcacatgctgtcagctattctaaaattgcatacattatgagctttttaaaagtccattatccaaattatttttacgcaaatattttaagtaatgttattggaagtgagaagaaaactgctcaaatgatagaagaagcaaaaaaacaaggtatcactatattgccaccgaacattaacgaaagtcattggttttataaaccttcccaagaaggcatttatttatcaattggtacaattaaaggtgttggttatcaaagtgtgaaagtgattgttgatgaacgttatcagaacggcaaatttaaagatttctttgattttgctagacgtataccgaagagagtcaaaacgagaaagttacttgaagcactgattttagtgggagcgtttgatgcttttggtaaaacacgttcaacgttgttgcaagctattgatcaagtgttggatggcgattttaaaacattggaacaagatggttttttatttgatattttaacgccaaaacagatgtatgaagataaagaagaattgcctgatgcacttattagtcagtacgaaaaagaatatttaggattttatgtttcgcaacacccagtagataaaaagtttgttgccaaacaatatttaacgatatttaaattgagtaacgcgcagaattataaacctatattagtacagtttgataaagttaaacaaattcgaactaaaaatggtcaaaatatggcattcgtcacattaaatgatggcattgaaactttagatggtgtgattttccctaatcagtttaaaaagtacgaagagttgttatcacataatgacttgtttatagttagcgggaaatttgaccatagaaagcaacaacgtcaactaattataaatgagattcagacattagccacttttgaagaacaaaaattagcatttgccaaacaaattataattagaaataaatcacaaatagatatgtttgaagagatgattaaagctacgaaagagaatgctaatgatgttgtgttatccttttatgatgaaacgattaaacaaatgactactttaggctatattaatcaaaaagatagtatgtttaataattttatacaatcctttaaccctagtgatattaggcttata

SEQ ID NO: 110 - protein, 1066 aa, Staphylococcus aureus
MVAYLNIHTAYDLLNSSLKIEDAVRLAVSENVDALAITDTNVLYGFPKFYDACIANNIKPIFGMTIYVTNGLNTVETVVLAKNNDGLKDLYQLSSEIKMNALEHVSFELLKRFSNNMIIIFKKVGDQHRDIVQVFETHNDTYMDHLSISIQGRKHVWIQNVCYQTRQDADTISALAAIRDNTKLDLIHDQEDFGAHFLTEKEINQLDINQEYLTQVDVIAQKCDAELKYHQSLLPQYETPNDESAKKYLWRVLVTQLKKLELNYDVYLERLKYEYKVITNMGFEDYFLIVSDLIHYAKTNDVMVGPGRGSSAGSLVSYLLGITTIDPIKFNLLFERFLNPERVTMPDIDIDFEDTRRERVIQYVQEKYGELHVSGIVTFGHLLARAVARDVGRIMGFDEVTLNEISSLIPHKLGITLDEAYQIDDFKEFVHRNHRHERWFSICKKLEGLPRHTSTHAAGIIINDHPLYEYAPLTKGDTGLLTQWTMTEAERIGLLKIDFLGLRNLSIIHQILTQVKKDLGINIDIEKIPFDDQKVFELLSQGDTTGIFQLESDGVRSVLKKLKPEHFEDIVAVTSLYRPGPMEEIPTYITRRHDPSKVQYLHPHLEPILKNTYGVIIYQEQIMQIASTFANFSYGEADILRRAMSKKNRAVLESERQHFIEGAKQNGYHEDISKQIFDLILKFADYGFPRAHAVSYSKIAYIMSFLKVHYPNYFYANILSNVIGSEKKTAQMIEEAKKQGITILPPNINESHWFYKPSQEGIYLSIGTIKGVGYQSVKVIVDERYQNGKFKDFFDFARRIPKRVKTRKLLEALILVGAFDAFGKTRSTLLQAIDQVLDGDFKTLEQDGFLFDILTPKQMYEDKEELPDALISQYEKEYLGFYVSQHPVDKKFVAKQYLTIFKLSNAQNYKPILVQFDKVKQIRTKNGQNMAFVTLNDGIETLDGVIFPNQFKKYEELLSHNDLFIVSGKFDHRKQQRQLIINEIQTLATFEEQKLAFAKQIIIRNKSQIDMFEEMIKATKENANDVVLSFYDETIKQMTTLGYINQKDSMFNNFIQSFNPSDIRLI

SEQ ID NO: 111 - DNA, 21 bp, Artificial
  Oligonucleotide Primer
tagaagatgccgtaagacttg

SEQ ID NO: 112 - DNA, 21 bp, Artificial
  Oligonucleotide Primer
atatcacgatgttgatcaccg

SEQ ID NO: 113 - DNA, 825 bp, Staphylococcus aureus
atgaagaaaaaagcgttactaccattatttttaggtattatggtctttttggctggttgtgactattctaaacctgaaaaacgtagtgggtttttctacaatacattcgtagatccaatgaaaaatgtattggattggttgggaaataacttattaaacgacaattatggtttagctattattatccttgtattggtaattcgtattattttattaccattcatgttgtcaaactataaaaatagtcatatgatgcgtcaaaaaatgaaagttgcaaagccagaagttgaaaaaattcaagaaaaagtgaaacgtgcgcgtacacaagaagaaaaaatggctgcaaaccaagaattaatgcaagtatataaaaagtatgacatgaacccgattaagagtatgttgggttgtttaccaatgctaatccaattaccaatcatcatgggattatactttgtacttaaagaccaacttgtagatggtttgtttaaatatccacacttcttatggttcgatttaggacgtcctgatatttggattacaattattgccggtgttttatactttatccaagcatatgtatcaagtaaaacgatgccagacgaacaacgtcaaatgggttacatgatgatggtcatttcaccaattatgattatctggatttcattaagctcagcatcagcacttggtttgtactggtcagtcagtgcggcgttccttgtagttcaaacacactttgcgaacatttattatgaaaaagtcgctaaaaaagaagtacaacctttcattgaagcgtatgaaagagagcacaacggcggcagcaat

SEQ ID NO: 114 - protein, 275 aa, Staphylococcus aureus
MKKKALLPLFLGIMVFLAGCDYSKPEKRSGFFYNTFVDPMKNVLDWLGNNLLNDNYGLAIIILVLVIRIILLPFMLSNYKNSHMMRQKMKVAKPEVEKIQEKVKRARTQEEKMAANQELMQVYKKYDMNPIKSMLGCLPMLIQLPIIMGLYFVLKDQLVDGLFKYPHFLWFDLGRPDIWITIIAGVLYFIQAYVSSKTMPDEQRQMGYMMMVISPIMIIWISLSSASALGLYWSVSAAFLVVQTHFANIYYEKVAKKEVQPFIEAYEREHNGGSN

SEQ ID NO: 115 - DNA, 21 bp, Artificial
  Oligonucleotide Primer
tatggtctttttggctggttg

SEQ ID NO: 116 - DNA, 21 bp, Artificial
  Oligonucleotide Primer
tttttcttcttgtgtacgcgc

SEQ ID NO: 117 - DNA, 807 bp, Staphylococcus aureus
atggatttttccaacttttttcaaaacctcagtacgttaaaaattgtaacgagtatccttgatttactgatagtttggtatgtactttatcttctcatcacggtctttaagggaactaaagcgatacaattacttaaagggatattagtaattgttattggtcagcagataagtatgatattgaacttgactgcaacatctaaattattcgatatcgttattcaatggggggtattagctttaatagtaatattccaaccagaaattagacgtgcgttagaacaacttggtagaggtagctttttaaaacgctatacttctaatacgtatagtaaagatgaagagaaattgattcaatcggtttcaaaggctgtgcaatatatggctaaaagacgtataggtgcattaattgtctttgaaaaagaaacaggtcttcaagattatattgaaacaggtattgcaatggattcaaatatttcgcaagaacttttaattaatgtctttatacctaacacacctttacatgatggtgcaatgattattcaaggcacgaagattgcagcagcagcaagttatttgccattgtctgatagtcctaagatatctaaaagtttgggtacaagacatagagctgcggttggtatttcagaagtatctgatgcatttaccgttattgtatctgaagaaactggtgatatttcggtaacatttgatggaaaattacgacgagacatttcaaacgaaatttttgaagagttgcttgctgaacattggtttggcacacgctttcaaaagaaaggtgtgaaa

SEQ ID NO: 118 - protein, 269 aa, Staphylococcus aureus
MDFSNFFQNLSTLKIVTSILDLLIVWYVLYLLITVFKGTKAIQLLKGILVIVIGQQISMILNLTATSKLFDIVIQWGVLALIVIFQPEIRRALEQLGRGSFLKRYTSNTYSKDEEKLIQSVSKAVQYMAKRRIGALIVFEKETGLQDYIETGIAMDSNISQELLINVFIPNTPLHDGAMIIQGTKIAAAASYLPLSDSPKISKSLGTRHRAAVGISEVSDAFTVIVSEETGDISVTFDGKLRRDISNEIFEELLAEHWFGTRFQKKGVK

SEQ ID NO: 119 - DNA, 21 bp, Artificial
  Oligonucleotide Primer
ctttatcttctcatcacggtc

SEQ ID NO: 120 - DNA, 21 bp, Artificial
  Oligonucleotide Primer
tgcacctatacgtcttttagc

SEQ ID NO: 121 - DNA, 606 bp, Staphylococcus aureus
atgatgataatcgtcatgttactactaagttatcttatcggcgctttcccaagtggattcgtaattggaaaattatttttcaaaaaagatattagacaatttggtagtggtaatactggcgctactaatagctttagagtattaggtcgtcctgcaggattcttggtaacatttctagatattttcaaagggttcataactgttttcttccctttatggttacaagttcacgcagatggccctattagtactttttttacaaatggtttaattgttggcttattcgctatacttggacacgtttatcctgtttatttaaaattccaaggtggcaaagctgttgcaactagtgcaggtgtcgtcttgggagtcaatccgatacttttactaatacttgcaattatcttctttattgtattgaagatttttaaatatgtttctttagcaagtatcgttgcagcaatttgctgtgtgattggctcgcttatcattcaagactatattttattagtcgttagtttcttagtttcaatcatattgataattagacatcgctctaatatcgcaaggatttttagaggcgaagaacctaaaataaaatggatg

SEQ ID NO: 122 - protein, 202 aa, Staphylococcus aureus
MMIIVMLLLSYLIGAFPSGFVIGKLFFKKDIRQFGSGNTGATNSFRVLGRPAGFLVTFLDIFKGFITVFFPLWLQVHADGPISTFFTNGLIVGLFAILGHVYPVYLKFQGGKAVATSAGVVLGVNPILLLILAIIFFIVLKIFKYVSLASIVAAICCVIGSLIIQDYILLVVSFLVSIILIIRHRSNIARIFRGEEPKIKWM

SEQ ID NO: 123 - DNA, 21 bp, Artificial
  Oligonucleotide Primer
gttatcttatcggcgctttcc

SEQ ID NO: 124 - DNA, 21 bp, Artificial
  Oligonucleotide Primer
gcactagttgcaacagctttg

SEQ ID NO: 125 - DNA, 1293 bp, Staphylococcus aureus
atgcagttaaatagtaatggttggcatgttgatgaccatattgttgtcgctgtttctacaggtattgatagtatgtgtttattgtatcaactactaaatgattataaagatagttatagaaaactaacatgcttacatgtcaatcatggcgttaggtcagcttcaattgaggaagccagatttttagaagcatactgcgaacgtcatcacatcgatttacatatcaaaaagttagatttgtcgcatagtctcgaccgaaataacagcattcagaatgaagctcgaattaaacgttacgaatggtttgatgaaatgatgaatgtattagaagcggatgtattgctaacggcgcatcatttggacgatcaattagaaactattatgtatcgtatttttaatgggaaatcaacacgtaataaactaggatttgatgagttatcgaagcgaaaaggttatcagatttatcgaccacttttagctgtctctaaaaaagaaataaaacaattccaagagagatatcatattccatattttgaagatgaatctaataaagataacaaatatgttagaaatgatattcgtaatagaattattccagctattgatgaaaataatcaacttaaagtatcgcatttattaaaattaaaacaatggcatgatgaacaatatgatattttgcaatattcagctaaacaatttattcaagaatttgtgaagtttgatgaacagtcaaaatatttagaggtttctagacaagcttttaataacttaccaaactcattaaagatggttgtgttggactgcctattatcaaagtattatgagttgtttaatattagtgctaaaacatacgaagagtggtttaaacaatttagtagtaagaaagcacaattcagtattaatctcacggataaatggataattcaaatcgcatatggtaaattaataataatggctaaaaataatggcgatacatattttagagttcaaacaattaaaaagccaggtaattatatttttaacaaatatcgattagagatacattctaatttaccaaaatgtttatttccgcttacagtgagaacacgacaaagtggcgatacatttaaactgaatgggcgcgatggttataagaaagtgaatcgcctgtttatagattgtaaagtgccacagtgggttcgggatcaaatgccaatcgtattggataaacaacagcgcattattgcggtaggagatttatatcaacaacaaacaataaaaaaatggattataattagtaaaaatggagatgaa

SEQ ID NO: 126 - protein, 431 aa, Staphylococcus aureus
MQLNSNGWHVDDHIVVAVSTGIDSMCLLYQLLNDYKDSYRKLTCLHVNHGVRSASIEEARFLEAYCERHHIDLHIKKLDLSHSLDRNNSIQNEARIKRYEWFDEMMNVLEADVLLTAHHLDDQLETIMYRIFNGKSTRNKLGFDELSKRKGYQIYRPLLAVSKKEIKQFQERYHIPYFEDESNKDNKYVRNDIRNRIIPAIDENNQLKVSHLLKLKQWHDEQYDILQYSAKQFIQEFVKFDEQSKYLEVSRQAFNNLPNSLKMVVLDCLLSKYYELFNISAKTYEEWFKQFSSKKAQFSINLTDKWIIQIAYGKLIIMAKNNGDTYFRVQTIKKPGNYIFNKYRLEIHSNLPKCLFPLTVRTRQSGDTFKLNGRDGYKKVNRLFIDCKVPQWVRDQMPIVLDKQQRIIAVGDLYQQQTIKKWIIISKNGDE

SEQ ID NO: 127 - DNA, 21 bp, Artificial
  Oligonucleotide Primer
aatggttggcatgttgatgac

SEQ ID NO: 128 - DNA, 21 bp, Artificial
  Oligonucleotide Primer
attgatcgtccaaatgatgcg

SEQ ID NO: 129 - DNA, 429 bp, Staphylococcus aureus
atgttcatgggagaatacgatcatcaattagatacaaaaggacgtatgattataccgtccaagtttcgttatgacttaaatgagcgttttattatcacaagaggccttgataaatgtttattcggttacactctagacgaatggcaacagattgaagagaaaatgaaaaccttacctatgacaaaaaaagacgcacgtaagtttatgcgtatgttcttctctggtgctgttgaagtagaacttgataagcaagggcgtattaacatccctcaaaacttgaggaaatacgctaatttaactaaagaatgtacagtaatcggtgtttcaaatcgtattgagatttgggatagagaaacttggaatgatttctatgaagaatctgaagaaagtttcgaagatattgctgaagatttaatagattttgatttt

SEQ ID NO: 130 - protein, 143 aa, Staphylococcus aureus
MFMGEYDHQLDTKGRMIIPSKFRYDLNERFIITRGLDKCLFGYTLDEWQQIEEKMKTLPMTKKDARKFMRMFFSGAVEVELDKQGRINIPQNLRKYANLTKECTVIGVSNRIEIWDRETWNDFYEESEESFEDIAEDLIDFDF

SEQ ID NO: 131 - DNA, 22 bp, Artificial
  Oligonucleotide Primer
ttcatgggagaatacgatcatc

SEQ ID NO: 132 - DNA, 22 bp, Artificial
  Oligonucleotide Primer
gtatttcctcaagttttgaggg

SEQ ID NO: 133 - DNA, 741 bp, Staphylococcus aureus
atgctagaggcacaattttttactgatactggacaacatagagataagaatgaagatgcgggtggtattttttataatcaaactaatcaacaacttttagttctgtgtgatggtatgggtggccataaagcaggagaagttgcaagtaaatttgttacagatgagttgaaatcccgttttgaagcggaaaatcttatagaacaacatcaagctgaaaattggttgcgtaataatataaaagatataaattttcagttatatcactatgcacaagaaaatgcagaatataaaggtatgggtacaacatgtgtttgtgcacttgtttttgaaaaatcagttgtgatagcaaatgtcggtgattctagagcctatgttattaatagtcgacaaattgaacaaattactagtgatcactcatttgttaatcatcttgttttaacgggtcaaattacgccggaagaagcatttacacatccacaacgtaatattattacgaaggtgatgggcacagataaacgtgtgagtccagatttgtttattaagcgattaaatttttatgattatttattattaaattcagatggattaactgattatgttaaagacaatgaaattaagcgtttgttagtaaaagaaggtacaatagaagatcatggtgatcaattaatgcaattggcattagataaccattcgaaagataacgttactttcatactcgcggctattgaaggtgataaagta

SEQ ID NO: 134 - protein, 247 aa, Staphylococcus aureus
MLEAQFFTDTGQHRDKNEDAGGIFYNQTNQQLLVLCDGMGGHKAGEVASKFVTDELKSRFEAENLIEQHQAENWLRNNIKDINFQLYHYAQENAEYKGMGTTCVCALVFEKSVVIANVGDSRAYVINSRQIEQITSDHSFVNHLVLTGQITPEEAFTHPQRNIITKVMGTDKRVSPDLFIKRLNFYDYLLLNSDGLTDYVKDNEIKRLLVKEGTIEDHGDQLMQLALDNHSKDNVTFILAAIEGDKV

SEQ ID NO: 135 - DNA, 21 bp, Artificial
  Oligonucleotide Primer
ctgatactggacaacatagag

SEQ ID NO: 136 - DNA, 21 bp, Artificial
  Oligonucleotide Primer
accgacatttgctatcacaac

SEQ ID NO: 137 - DNA, 567 bp, Staphylococcus aureus
atgaaaaagatagtactttacggcggtcagtttaaccctatccatactgcacatatgatagtagctagcgaagtatttcatgaattacagccagatgaattttattttttacctagttttatgtctccattgaaaaagcaccatgattttatagacgttcagcacagattaacaatgatacagatgattatcgacgagcttggttttggagatatttgtgacgatgaaattaaacgtggtggtcaaagttatacctatgacacgatcaaggcattcaaggagcaacacaaagacagtgagttgtactttgttattgggacggatcagtataaccaactagagaaatggtatcaaattgaatacttaaaagaaatggttacttttgtagttgtaaatcgagacaaaaatagtcaaaatgttgaaaatgctatgattgcaattcagatacctagggtagatataagttcgacaatgattcgacaaagagttagtgaagggaaatctatccaagttcttgttcctaaatccgttgaaaactatattaagggggaaggattatatgaacat

SEQ ID NO: 138 - protein, 189 aa, Staphylococcus aureus
MKKIVLYGGQFNPIHTAHMIVASEVFHELQPDEFYFLPSFMSPLKKHHDFIDVQHRLTMIQMIIDELGFGDICDDEIKRGGQSYTYDTIKAFKEQHKDSELYFVIGTDQYNQLEKWYQIEYLKEMVTFVVVNRDKNSQNVENAMIAIQIPRVDISSTMIRQRVSEGKSIQVLVPKSVENYIKGEGLYEH

SEQ ID NO: 139 - DNA, 21 bp, Artificial
  Oligonucleotide Primer
gtttaaccctatccatactgc

SEQ ID NO: 140 - DNA, 21 bp, Artificial
  Oligonucleotide Primer
ctagttggttatactgatccg

SEQ ID NO: 141 - DNA, 393 bp, Staphylococcus aureus
atggtaacattatttacttcaccaagttgcacatcttgccgtaaagcgaaagcatggttacaagaacatgacattccgtatacggagcgtaatattttttctgaacatttaacaattgatgaaattaagcaaatattaaaaatgactgaagacggtactgatgaaatcatttctacacgttctaaaacataccaaaaattaaatgttgatattgattcactaccattacaagacttatattcaatcattcaagataatcctggcttattacgtcgtccaattattttagataataaacgactacaagttggttataatgaggacgagattcgacgtttcttacctagaaaagttcgtacgttccaattacaagaagcacaacgtatggttgac

SEQ ID NO: 142 - protein, 131 aa, Staphylococcus aureus
MVTLFTSPSCTSCRKAKAWLQEHDIPYTERNIFSEHLTIDEIKQILKMTEDGTDEIISTRSKTYQKLNVDIDSLPLQDLYSIIQDNPGLLRRPIILDNKRLQVGYNEDEIRRFLPRKVRTFQLQEAQRMVD

SEQ ID NO: 143 - DNA, 21 bp, Artificial
  Oligonucleotide Primer
atttacttcaccaagttgcac

SEQ ID NO: 144 - DNA, 21 bp, Artificial
  Oligonucleotide Primer
taattggacgacgtaataagc

SEQ ID NO: 145 - DNA, 20 bp, Artificial
  Oligonucleotide Primer
gggcccaagcttagtgatgg

(19) United States
(12) Patent Application Publication
(10) Pub. No.: US 2003/0180821 A1
(43) Pub. Date: Sep. 25, 2003

(54) Antimicrobial methods and materials

(75) Inventors: Che-Shen C. Tomich, Kalamazoo, MI (US); Cheryl L. Quinn, Kalamazoo, MI (US); Staffan Arvidson, Akersberga (SE); John E. Mott, Kalamazoo, MI (US); Douglas W. Harris, Portage, MI (US)

Correspondence Address: MUETING, RAASCH & GEBHARDT, P.A., P.O. BOX 581415, MINNEAPOLIS, MN, 55458, US

(73) Assignee: PHARMACIA & UPJOHN COMPANY, Kalamazoo, MI (US)

(21) Appl. No.: 10/429,094

(22) Filed: May 2, 2003

Related U.S. Application Data
(63) Continuation of application No. 09/966,521, filed on Sep. 28, 2001.

(51) Int. Cl. G01N 33/554; G01N 33/569; C12Q 1/18

(52) U.S. Cl. 435/7.32; 435/32

(57) ABSTRACT

The present invention provides methods of identifying agents that bind gene products critical for the survival of microbes, preferably Staphylococcus microbes, including agents that interfere with the expression of such gene products and agents that interfere with the function of such gene products.

### BACKGROUND

The staphylococci, of which Staphylococcus aureus is the most important human pathogen, are hardy, gram-positive bacteria that colonize the skin of most humans. Staphylococcal strains that produce coagulase are designated S. aureus; other clinically important coagulase-negative staphylococci are S. epidermidis and S. saprophyticus. When the skin or mucous membrane barriers are disrupted, staphylococci can cause localized and superficial infections that are commonly harmless and self-limiting. However, when staphylococci invade the lymphatics and the blood, potentially serious complications may result, such as bacteremia, septic shock, and serious metastatic infections, including endocarditis, arthritis, osteomyelitis, pneumonia and abscesses in virtually any organ. Certain strains of S. aureus produce toxins that cause skin rashes, food poisoning, or multisystem dysfunction (as in toxic shock syndrome). S. aureus and S. epidermidis together have become the most common cause of nosocomial non-urinary tract infection in U.S. hospitals. They are the most frequently isolated pathogens in both primary and secondary bacteremias and in cutaneous and surgical wound infections. See generally Harrison's Principles of Internal Medicine, 13th ed., Isselbacher et al., eds., McGraw-Hill, New York (1994), particularly pages 611-617.

Transient colonization of the nose by S. aureus is seen in 70 to 90 percent of people, of which 20 to 30 percent carry the bacteria for relatively prolonged periods of time. Independent colonization of the perineal area occurs in 5 to 20 percent of people. Higher carriage rates of S. aureus have been documented in persons with atopic dermatitis, hospital employees, hospitalized patients, patients whose care requires frequent puncture of the skin, and intravenous drug abusers.

Infection by staphylococci usually results from a combination of bacterial virulence factors and a diminution in host defenses. Important microbial factors include the ability of the staphylococcus to survive under harsh conditions, its cell wall constituents, the production of enzymes and toxins that promote tissue invasion, its capacity to persist intracellularly in certain phagocytes, and its potential to acquire resistance to antimicrobial agents. Important host factors include an intact mucocutaneous barrier, an adequate number of functional neutrophils, and removal of foreign bodies or dead tissue.

Cell wall components of S. aureus include a large peptidoglycan complex that confers rigidity on the organism and enables it to survive under unfavorable osmotic conditions, a unique teichoic acid linked to peptidoglycan, and protein A, which is found both attached to peptidoglycan over the outermost parts of the cell and released in soluble form. Proteins designated femA and femB are involved in the formation of cell wall peptidoglycan pentaglycine cross-bridges and are factors in methicillin resistance (Berger-Bachi et al., Mol. Gen. Genet., 219, 263-269 (1989)). S. aureus also has specific receptors for laminin and fibronectin that may mediate the organism's spread through the bloodstream to other tissues. Both peptidoglycan and teichoic acid are capable of activating the complement cascade via the alternative pathway. S. aureus also appears to activate tissue factor in the coagulation pathway.

Certain enzymes produced by S. aureus may play a role in virulence. Catalase degrades hydrogen peroxide and may protect the organism during phagocytosis. Coagulase is present in both soluble and cell-bound forms and causes plasma to clot by formation of thrombin-like material. The high correlation between coagulase production and virulence suggests that this substance is important in the pathogenesis of staphylococcal infections, but its precise role as a determinant of pathogenicity has not been determined. Many strains also produce hyaluronidase, an enzyme that degrades hyaluronic acid in the connective tissue matrix and that may promote spreading of infection. A trypsin-like protease from some strains enhances influenza virus infection by proteolytic cleavage of the viral precursor hemagglutinin into its active fragments and may contribute to the morbidity of such co-infections. S. aureus produces numerous extracellular exotoxins that have been implicated in disease processes. The exfoliatin toxins A and B, the staphylococcal enterotoxins, and the toxic shock syndrome toxin, TSST-1, belong to the growing family of microbial superantigens that activate T cells and monocytes/macrophages, resulting in the production of cytokines that mediate local or systemic effects depending on the amount of toxin formed, the immune status of the host, and the access of the toxin to the circulation. The exfoliatin toxins mediate the dermatologic manifestations of the staphylococcal scalded-skin syndrome and bullous impetigo. These toxins cause intraepidermal cleavage of the skin at the stratum granulosum, leading to bullae formation and denudation. Seven distinct enterotoxins (A, B, C1, C2, C3, D, and E) have been implicated in food poisoning due to S. aureus. These toxins enhance intestinal peristalsis and appear to induce vomiting by a direct effect on the central nervous system. Toxic shock syndrome (TSS) is most commonly mediated by TSST-1, which is present in 5 to 25 percent of clinical isolates of S. aureus. TSS is also mediated less frequently by enterotoxin B and, rarely, enterotoxin C1.

S. aureus produces other toxins whose role in virulence is incompletely understood. Four different red blood cell hemolysins, which are designated alpha, beta, gamma, and delta toxins, have been identified. Alpha toxin also causes necrosis of the skin when injected subcutaneously into animals, while delta toxin also inhibits water absorption in the intestines and may play a role in the acute watery diarrhea seen in some cases of staphylococcal infection. Leukocidin lyses granulocyte and macrophage membranes by producing membrane pores permeable to cations.

The agr, xpr, sae and sar coding sequences have been identified as being involved in the regulation of staphylococcal exotoxins. See U.S. Pat. No. 5,587,228 and International Patent Publication Nos. WO 96/10579 and WO 97/11690. Of interest is the report in WO 97/11690 of screening for inhibitors of these regulatory systems.

Staphylococci can invade the skin or mucosa through plugged hair follicles and sebaceous glands or areas traumatized by burns, wounds, abrasions, insect bites, or dermatitis. Staphylococci often colonize prosthetic devices and intravenous catheters; S. aureus infection of the vascular access site is a major cause of morbidity and death among patients on hemodialysis. Colonization and invasion of the lungs may occur with endotracheal intubation, or when the lungs' clearance mechanisms are depressed, e.g., after viral infections, after aspiration, or in patients with cystic fibrosis. Mucosal damage to the gastrointestinal tract following cytotoxic chemotherapy or radiotherapy predisposes to invasion from that site.

Once the skin or mucosa have been breached, local bacterial multiplication is accompanied by inflammation, neutrophil accumulation, tissue necrosis, thrombosis and fibrin deposition at the site of infection. Later, fibroblasts create a relatively avascular wall about the area. When host mechanisms fail to contain the cutaneous or submucosal infection, staphylococci may enter the lymphatics and the bloodstream. Common sites of metastatic spread include the lungs, kidneys, cardiac valves, myocardium, liver, spleen, bones and brain.

Bacteremia due to S. aureus may arise from any local infection, at either extravascular (cutaneous infections, burns, cellulitis, osteomyelitis, arthritis) or intravascular foci (intravenous catheters, dialysis access sites, intravenous drug abuse). Commonly, the disease progresses more slowly, with hectic fever and metastatic abscess formation. Rarely, patients with bacteremia die within 12 to 24 hours with high fever, tachycardia, cyanosis, and vascular collapse. Disseminated intravascular coagulation may produce a disease mimicking meningococcemia.

A major complication of S. aureus bacteremia is endocarditis. S. aureus is the second most common cause of endocarditis and the most common cause among drug addicts. The disease is typically acute, with high fever, progressive anemia, and frequent embolic and extracardiac septic complications. Valve ring and myocardial abscesses are common. The mortality rate is 20 to 30 percent.

Staphylococcal scalded-skin syndrome (SSSS) is a generalized exfoliative dermatitis that is a complication of infection by exfoliatin toxin-producing strains of S. aureus. The disease typically occurs in newborns (Ritter's disease) and in children under the age of five. A scarlatiniform rash begins in the perioral area, becomes generalized over the trunk and extremities, and finally desquamates. The disease may consist of rash alone (staphylococcal scarlet fever), or large, flaccid bullae develop that may be localized (more common in adults) or generalized. The bullae burst, resulting in red, denuded skin resembling a burn. Most adults with SSSS are immunosuppressed or have renal insufficiency. Blood cultures are frequently positive, and mortality is significant.

Toxic shock syndrome (TSS) is a multisystem disease mediated by toxins (generally TSST-1, and less frequently enterotoxins B and C1) produced by certain strains of S. aureus. It was first described in children, but in 1980 became epidemic among young women, with onset during menstruation. The diagnosis of TSS is based on clinical criteria that include high fever, a diffuse rash that desquamates on the palms and soles over the subsequent one or two weeks, hypotension that may be orthostatic, and evidence of involvement in three or more organ systems. Such involvement commonly includes gastrointestinal dysfunction (vomiting or diarrhea), renal or hepatic insufficiency, mucous membrane hyperemia, thrombocytopenia, myalgias with elevated creatine phosphokinase (CK) levels, and disorientation with a normal cerebrospinal fluid examination. The mortality rate of TSS is three percent.

S. aureus causes approximately three percent of community-acquired bacterial pneumonias. This disease occurs sporadically except during influenza outbreaks, when staphylococcal pneumonia is relatively more common, although still less frequent than pneumococcal pneumonia. Primary staphylococcal pneumonia in infants and children frequently presents with high fever and cough. Multiple thin-walled abscesses are seen on the chest X-ray, and empyema formation is common. In older children and healthy adults, staphylococal pneumonia is generally preceded by an influenza-like respiratory infection. Onset of staphylococcal involvement is abrupt, with chills, high fever, progressive dyspnea, cyanosis, cough, pleural pain, and sometimes bloody sputum. Staphylococcal pneumonia is seen more frequently in patients with cystic fibrosis, in intubated patients in intensive care units and in debilitated patients who are prone to aspiration.

S. aureus is responsible for the majority of cases of acute osteomyelitis. Although the disease is most common in people under the age of 20, it is becoming increasingly prevalent in adults over 50, particularly with involvement of the spine. A primary portal of entry is frequently not identified, although many patients give a history of preceding trauma to the involved area. Once established, infection spreads through the bone to the periosteum or along the marrow cavity. Rarely, the joint capsule is penetrated, producing pyogenic arthritis. Osteomyelitis in children may present as an acute process beginning abruptly with chills, high fever, nausea, vomiting, and progressive pain at the site of bony involvement.

S. aureus causes 1 to 9 percent of cases of bacterial meningitis and 10 to 15 percent of brain abscesses. Most commonly, the bacteria are spread from a focus outside the central nervous system, typically from infective endocarditis, by extension from a paraspinal or parameningeal abscess, or by nosocomial infection following neurosurgical procedures. Over 50 percent of epidural abscesses are due to S. aureus; up to half of these cases may be associated with vertebral osteomyelitis. Patients present with either acute or chronic back pain, usually with low-grade fever and malaise. The onset of radicular pain is an ominous sign that the disease may progress to neurologic dysfunction and ultimate paralysis.

Antimicrobial resistance by staphylococci favors their persistence in the hospital environment. Over 90 percent of both hospital and community strains of S. aureus causing infection are resistant to penicillin. This resistance is due to the production of β-lactamase enzymes; the nucleotides encoding these enzymes are usually carried by plasmids. Infections due to organisms with such acquired resistance can sometimes be treated with penicillinase-resistant β-lactam antimicrobial agents. However, the true penicillinase-resistant S. aureus organisms, called methicillin-resistant S. aureus (MILSA), are resistant to all the β-lactam antimicrobial agents as well as the cephalosporins. MRSA resistance is chromosomally mediated and involves production of an altered penicillin-binding protein (PBP 2a or PBP 2′) with a low binding affinity for β-lactams. MRSA frequently also have acquired plasmids mediating resistance to erythromycin, tetracycline, chloramphenicol, clindamycin, and aminoglycosides. MRSA have become increasingly common worldwide, particularly in tertiary-care referral hospitals. In the United States, approximately 5 percent of hospital isolates of S. aureus are methicillin-resistant.

Thus, there continues to exist a need for new agents useful for treating bacterial infections, particularly those caused by antibiotic-resistant bacteria, and for methods of identifying such new agents. Such methods ideally would identify agents that are unrelated to existing antimicrobials and that target different aspects of staphylococcal invasion of and replication in the host, compared to existing antimicrobials.

### SUMMARY OF THE INVENTION

The present invention provides a method for identifying an agent that binds a polypeptide. The method includes contacting a polypeptide and an agent to form a mixture, and determining whether the agent binds the polypeptide. In some aspects, the polypeptide may be encoded by a coding sequence having a nucleotide sequence of SEQ ID NO:7, 21, 23, 25, 27, 29, 31, 33, 109, 113, 117, 121, 125, 129, 133, 137, or 141. In another aspect of the invention, the polypeptide is encoded by an essential coding sequence having a nucleotide sequence with at least about 57 percent structural similarity to a nucleotide sequence of SEQ ID NO:7, 21, 23, 25, 27, 29, 31, 33, 109, 113, 117, 121, 125, 129, 133, or 137. In another aspect, the polypeptide is encoded by a critical coding sequence having a nucleotide sequence with at least about 57 percent structural similarity to the nucleotide sequence SEQ ID NO:141.

Determining whether the agent binds the polypeptide may be accomplished by conducting an enzyme assay, a binding assay, or a ligand binding assay. The method may further include determining whether the agent decreases the growth rate of a microbe. This includes contacting a microbe with the agent, incubating the microbe and the agent under conditions suitable for growth of the microbe that is not contacted with the agent, and determining the growth rate of the microbe contacted with the agent. A decrease in growth rate compared to the microbe that is not contacted with the agent indicates the agent decreases the growth rate of the microbe. The microbe may be in vitro or in vivo, and the microbe may be a Staphylococcus aureus. The present invention also provides an agent identified by the method.

The present invention also provides a method for identifying an agent that decreases the growth rate of a microbe. The method includes contacting a microbe with an agent, incubating the microbe and the agent under conditions suitable for growth of the microbe that is not contacted with the agent, and determining the growth rate of the microbe contacted with the agent. A decrease in growth rate compared to the microbe that is not contacted with the agent indicates the agent decreases the growth rate of the microbe. In some aspects, the polypeptide may be encoded by a coding sequence having a nucleotide sequence of SEQ ID NO:7, 21, 23, 25, 27, 29, 31, 33, 109, 113, 117, 121, 125, 129, 133, 137, or 141. In another aspect of the invention, the polypeptide is encoded by an essential coding sequence having a nucleotide sequence with at least about 57 percent structural similarity to a nucleotide sequence of SEQ ID NO:7, 21, 23, 25, 27, 29, 31, 33, 109, 113, 117, 121, 125, 129, 133, or 137. In another aspect, the polypeptide is encoded by a critical coding sequence having a nucleotide sequence with at least about 57 percent structural similarity to the nucleotide sequence SEQ ID NO:141. The microbe may be in vitro or in vivo, and the microbe may be a Staphylococcus aureus. The present invention also provides an agent identified by the method.

Also provided by the present invention is a method for decreasing the growth rate of a microbe. The method includes contacting a microbe with an agent that binds to a polypeptide. In some aspects, the polypeptide may be encoded by a coding sequence having a nucleotide sequence of SEQ ID NO:7, 21, 23, 25, 27, 29, 31, 33, 109, 113, 117, 121, 125, 129, 133, 137, or 141. In another aspect of the invention, the polypeptide is encoded by an essential coding sequence having a nucleotide sequence with at least about 57 percent structural similarity to a nucleotide sequence of SEQ ID NO:7, 21, 23, 25, 27, 29, 31, 33, 109, 113, 117, 121, 125, 129, 133, or 137. In another aspect, the polypeptide is encoded by a critical coding sequence having a nucleotide sequence with at least about 57 percent structural similarity to the nucleotide sequence SEQ ID NO:141. The microbe may be in vitro or in vivo, and the microbe may be a Staphylococcus aureus.

The present invention provides a method for making an S. aureus with reduced virulence. The method includes altering a coding sequence in an S. aureus to include a mutation, and determining if the S. arueus having the mutation has reduced virulence compared to an S. arueus that does not have the mutation. The coding sequence that is altered to include a mutation may include a nucleotide sequence of SEQ ID NO:7, 21, 23, 25, 27, 29, 31, 33, 109, 113, 117, 121, 125, 129, 133, 137, or 141. In another aspect of the invention, the coding sequence that is altered to include a mutation is an essential coding sequence that may include a nucleotide sequence having at least about 57 percent structural similarity to a nucleotide sequence of SEQ ID NO:7, 21, 23, 25, 27, 29, 31, 33, 109, 113, 117, 121, 125, 129, 133, or 137. In yet another aspect, the coding sequence that is altered to include a mutation is a critical coding sequence that may include a nucleotide sequence having at least about 57 percent structural similarity to a nucleotide sequence of SEQ ID NO:141. The invention also provides the S. aureus having reduced virulence, and a vaccine composition that includes the S. aureus having reduced virulence.

The present invention further provides isolated polynucleotides. A polynucleotide may include a nucleotide sequence of SEQ ID NO:7, 21, 23, 25, 27, 29, 31, 33, 109, 113, 117, 121, 125, 129, 133, 137, or 141. In another aspect, a polynucleotide may include a nucleotide sequence having at least about 57 percent structural similarity with a nucleotide sequence of SEQ ID NO:7, 21, 23, 25, 27, 29, 31, 109, 113, 117, 121, 125, 129, 133, or 137, wherein the isolated polynucleotide includes an essential coding sequence. In yet another aspect, a polynucleotide may include a nucleotide sequence having at least about 57 percent structural similarity with a nucleotide sequence of SEQ ID NO:141, wherein the isolated polynucleotide includes a critical coding sequence. A polynucleotide may also consist essentially of the above described nucleotide sequences, and the polynucleotide may optionally further include from zero to up to about 5,000 nucleotides upstream and/or downstream of the nucleotide sequence.

Also provided by the present invention are isolated polypeptides that include an amino acid sequence of SEQ ID NO:2, 4, 6, 8, 10, 12, 14, 16, 18, 20, 22, 24, 26, 28, 30, 32, 34, 110, 114, 118, 122, 126, 130, 134, 138, or 142.

Definitions

As used herein, the term “agent” refers to chemical compounds, including, for instance, an organic compound, an inorganic compound, a metal, a polypeptide, a non-ribosomal polypeptide, a polyketide, or a peptidomimetic compound that binds to a particular polypeptide.

As used herein, the term “polypeptide” refers to a polymer of amino acids and does not refer to a specific length of a polymer of amino acids. Thus, for example, the terms peptide, oligopeptide, protein, and enzyme are included within the definition of polypeptide. This term also includes post-expression modifications of the polypeptide, for example, glycosylations, acetylations, phosphorylations and the like.

The term “binds to a polypeptide” refers to a condition of proximity between an agent and a polypeptide. The association may be non-covalent, wherein the juxtaposition is energetically favored by hydrogen bonding, van der Waals forces, or electrostatic interactions, or it may be covalent.

As used herein, growth of a microbe “in vitro” refers to growth, for instance, in a test tube or on an agar plate. Growth of a microbe “in vivo” refers to growth, for instance, in a cultured cell or in an animal.

As used herein, the term “microbe” and “bacteria” are used interchangeably and include single celled prokaryotic and lower eukaryotic (e.g., fungi) organisms, preferably prokaryotic organisms.

### BRIEF DESCRIPTION OF THE FIGURES

FIGS. 1a-z. The nucleotide sequence of the coding sequences of 26 S. aureus coding sequences (SEQ ID NO: 1, 3, 5, 7, 9, 11, 13, 15, 17, 19, 21, 23, 25, 27, 29, 31, 33, 109, 113, 117, 121, 125, 129, 133, 137, and 141), the predicted sequence of the peptide (SEQ ID NO: 2, 4, 6, 8, 10, 12, 14, 16, 18, 20, 22, 24, 26, 28, 30, 32, 34, 110, 114, 118, 122, 126, 130, 134, 138, or 142, respectively) encoded by each coding sequence, and primer pairs used for preparing fragments for insertion into a temperature sensitive plasmid (SEQ ID NO: 35-68, 111-112, 115-116, 119-120, 123-124, 127-128, 131-132, 135-136, 139-140, and 143-144). The two underlined sequences in each coding sequence correspond to the primers listed below the coding sequence.

FIGS. 2a-i. The nucleotide sequence of each of 9 S. aureus coding sequences to be cloned for expression in E. coli (SEQ ID NO:69, 71, 73, 75, 77, 79, 81, 83, and 85), the predicted sequence of the peptide (SEQ ID NO:70, 72, 74, 76, 78, 80, 82, 84, and 86, respectively) encoded by each coding sequence after insertion into the appropriate expression plasmid, and the sequence of the primer pair (SEQ ID NO:91-108) used to clone the S. aureus coding sequences by amplification. The top primer and bottom primer of each primer pair is the forward primer and the reverse primer, respectively. The underlined ATGG in SEQ ID NO:69, 73, 75, 77, and 79 shows the location of a portion of the NcoI restriction site added to the coding sequence by the forward primer for cloning into the expression vector pQE-60. The underlined AGATCT in SEQ ID NOS:69, 71, 73, 75, 79, 81, 83, and 85 shows the location of the BglII restriction site added to the coding sequence by the reverse primer. The underlined GGATCT in SEQ ID NO:77 shows the location of the ligation of the digested BamHI restriction site of the amplified fragment with the digested BglII restriction site of the vector.

FIG. 3. Nucleotide sequence (SEQ ID NO:87) and the predicted amino acid sequence (SEQ ID NO:88) of the S. aureus uridylate kinase.

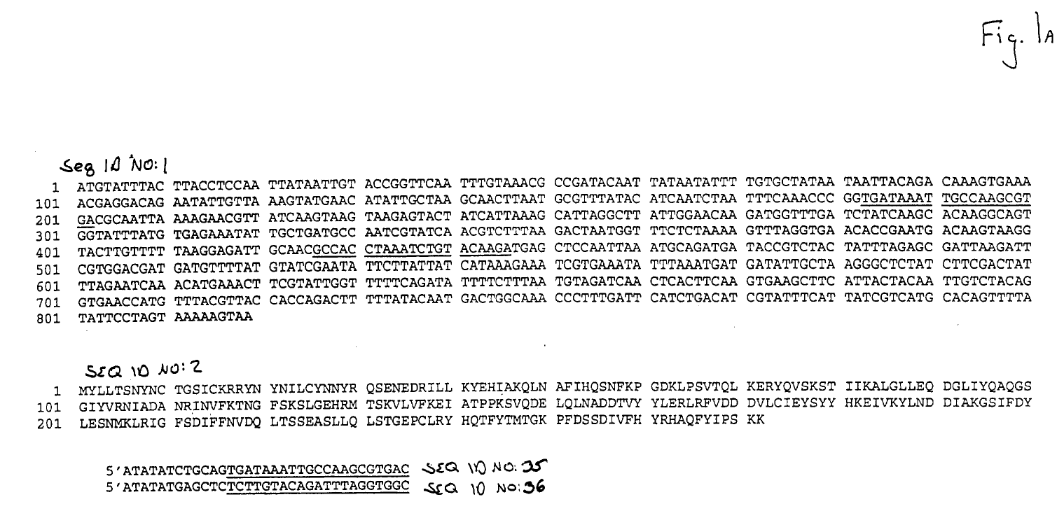

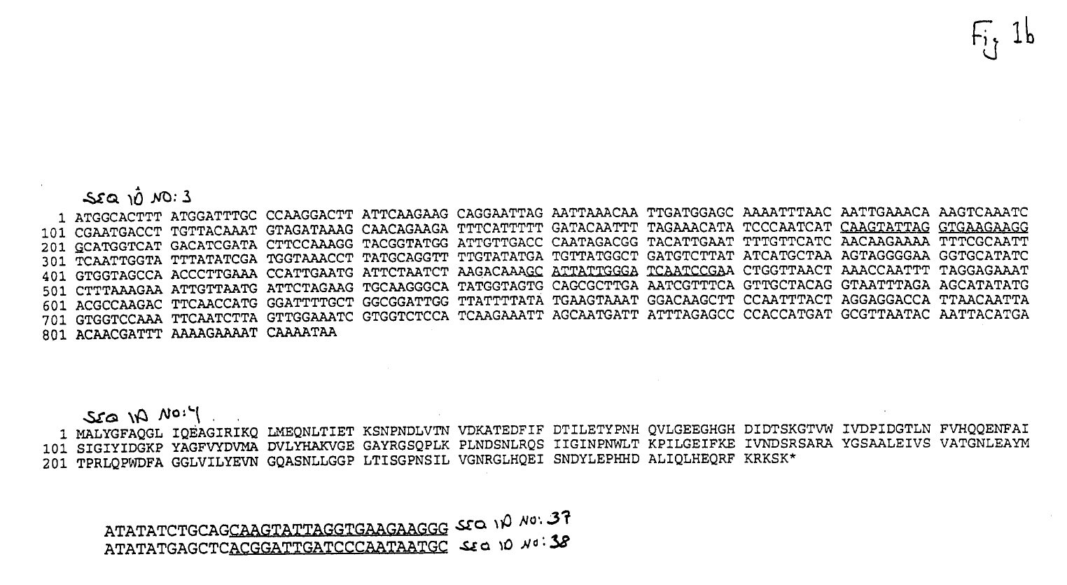

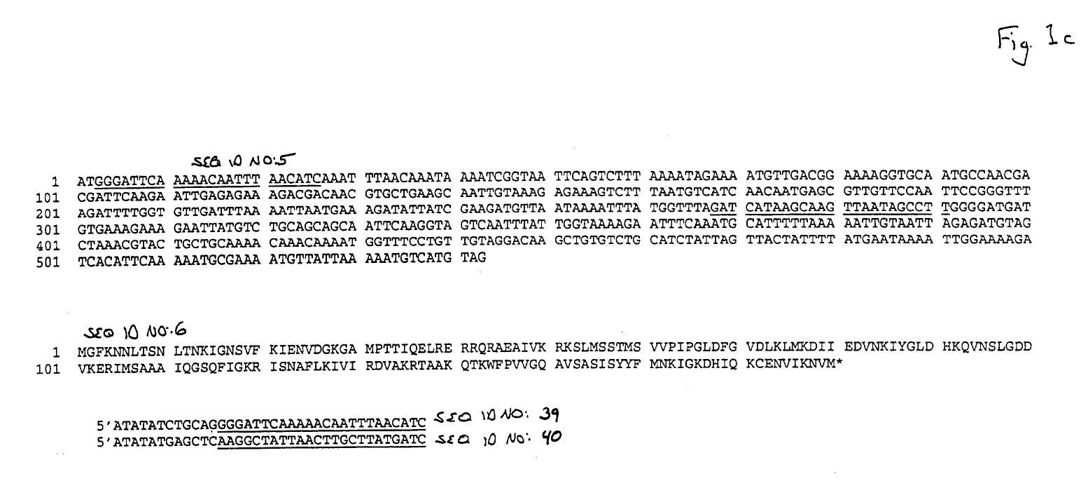

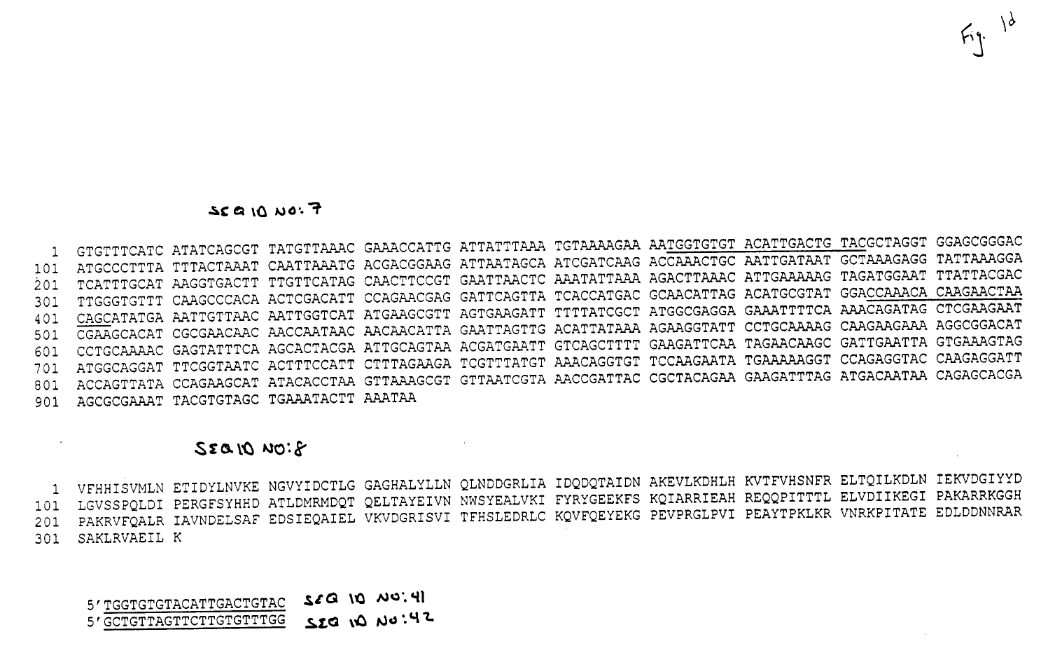

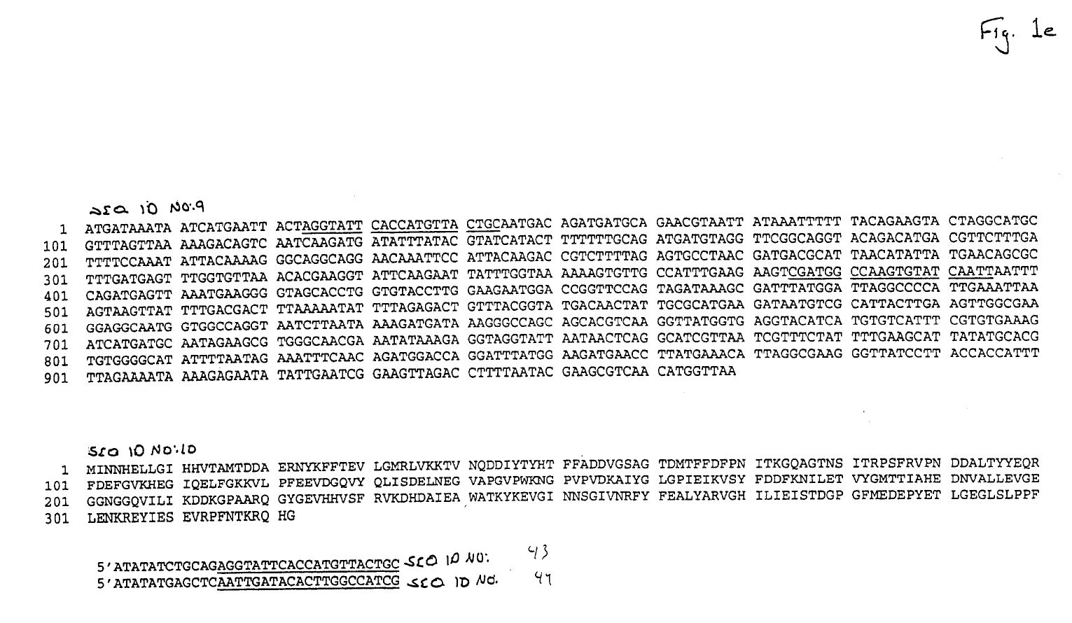

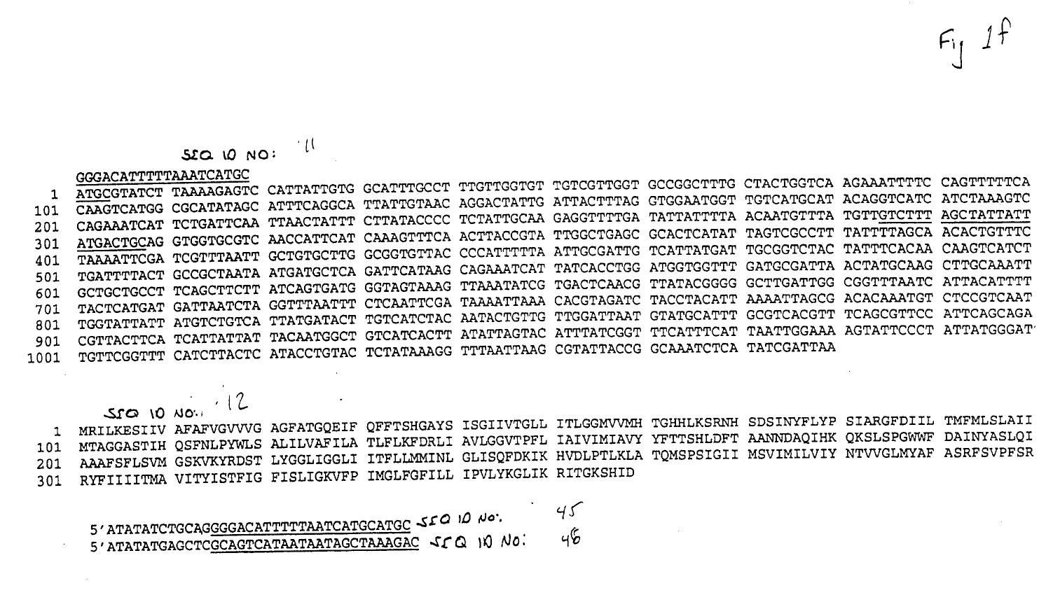

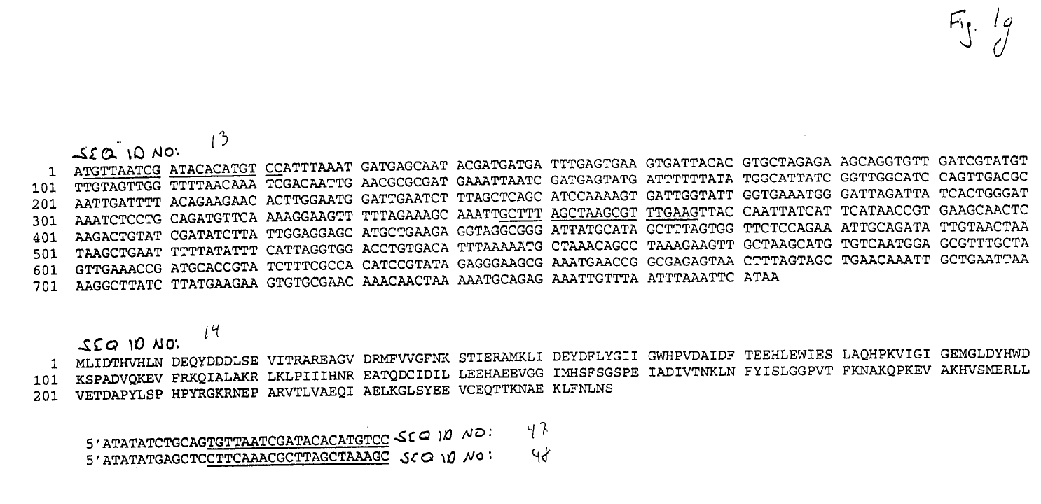

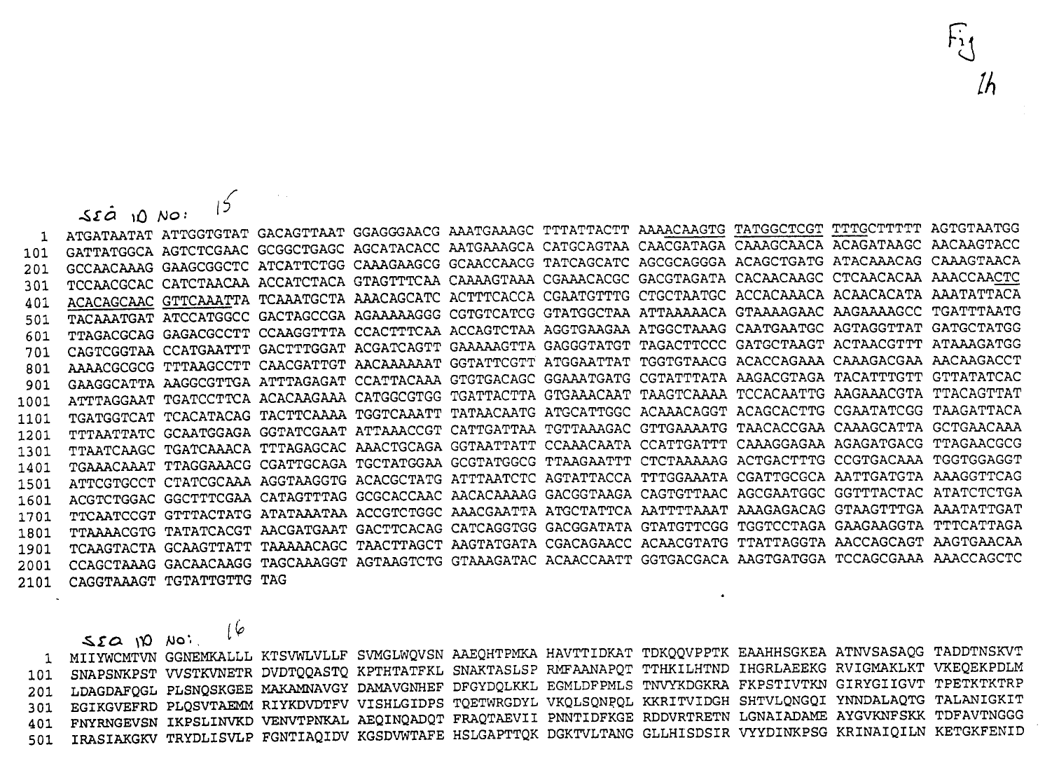

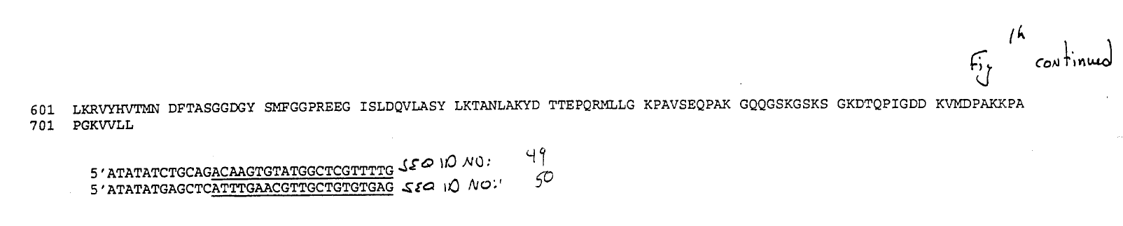

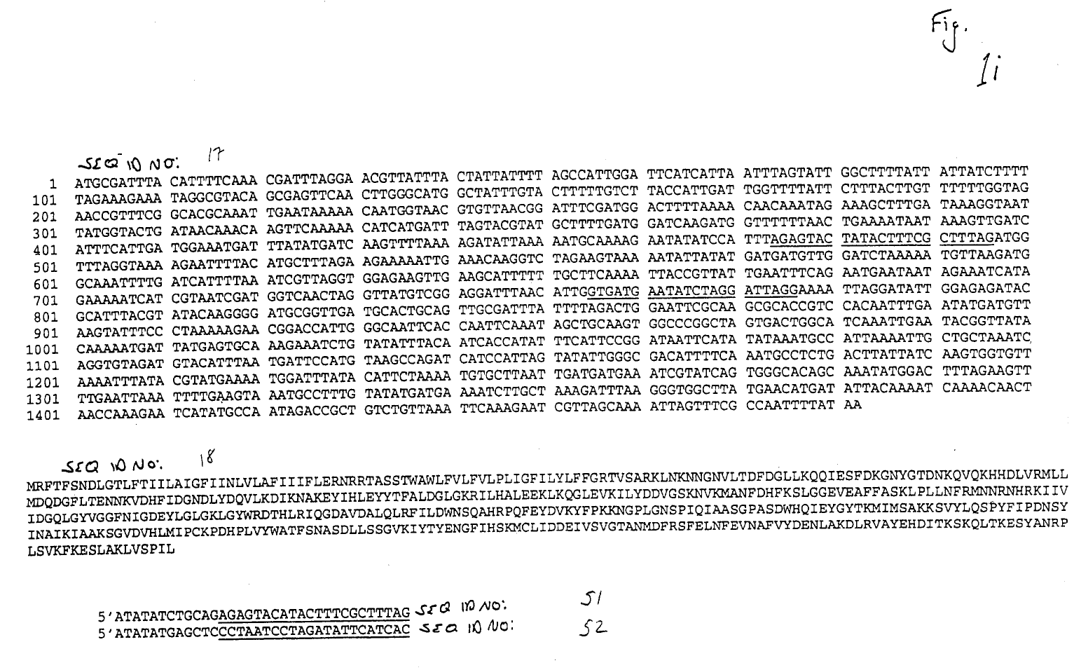

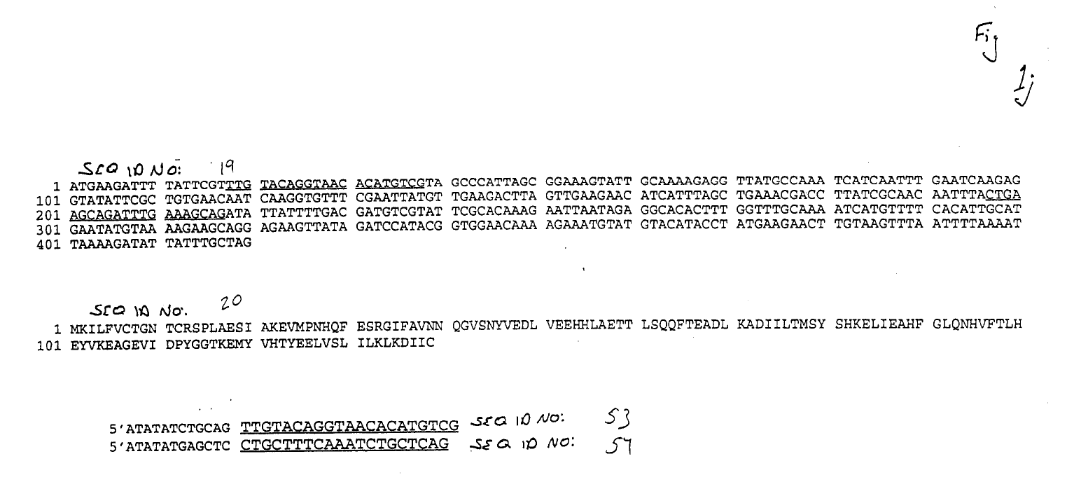

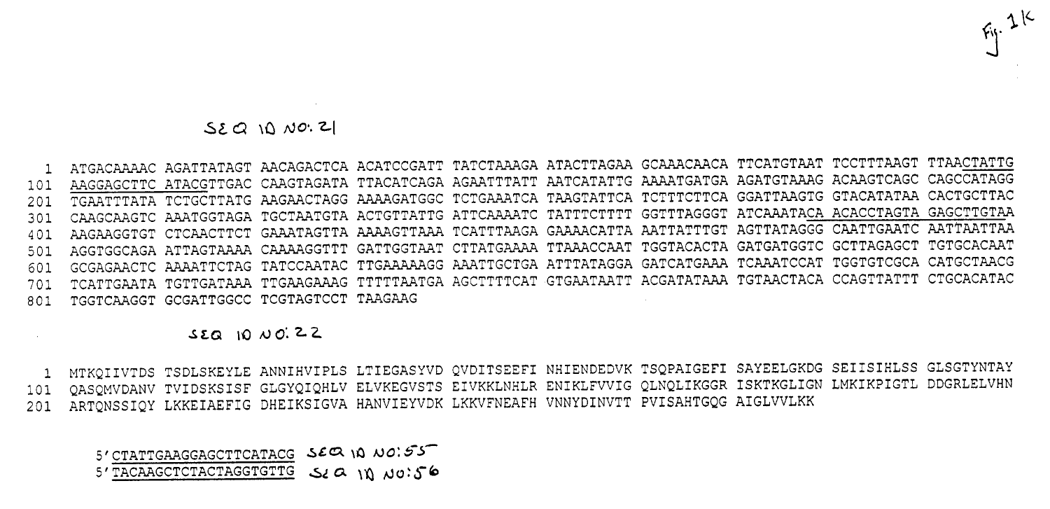

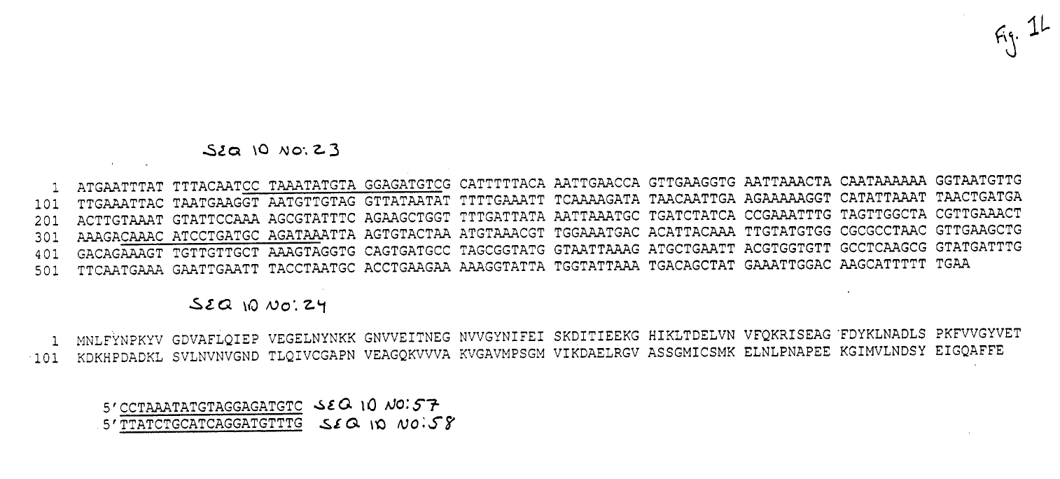

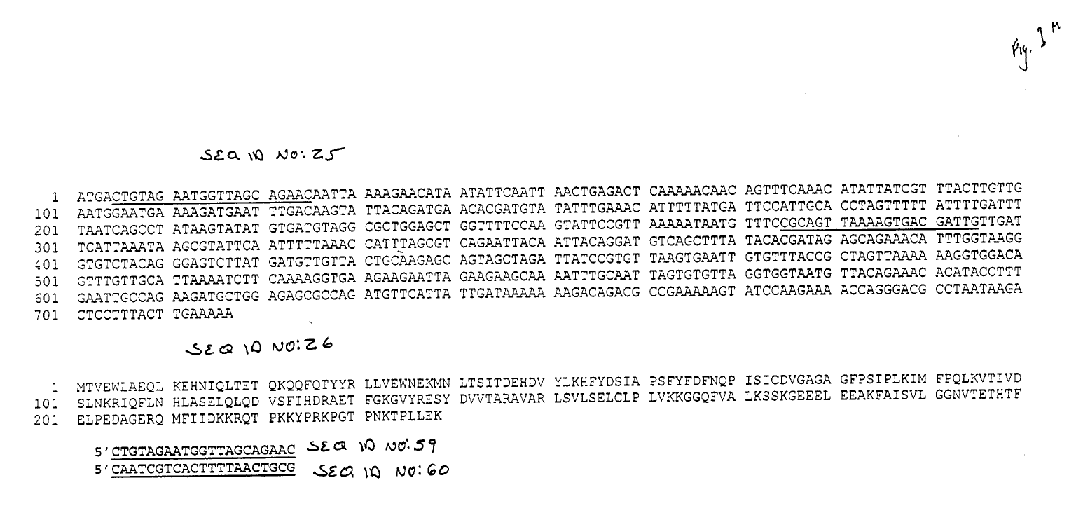

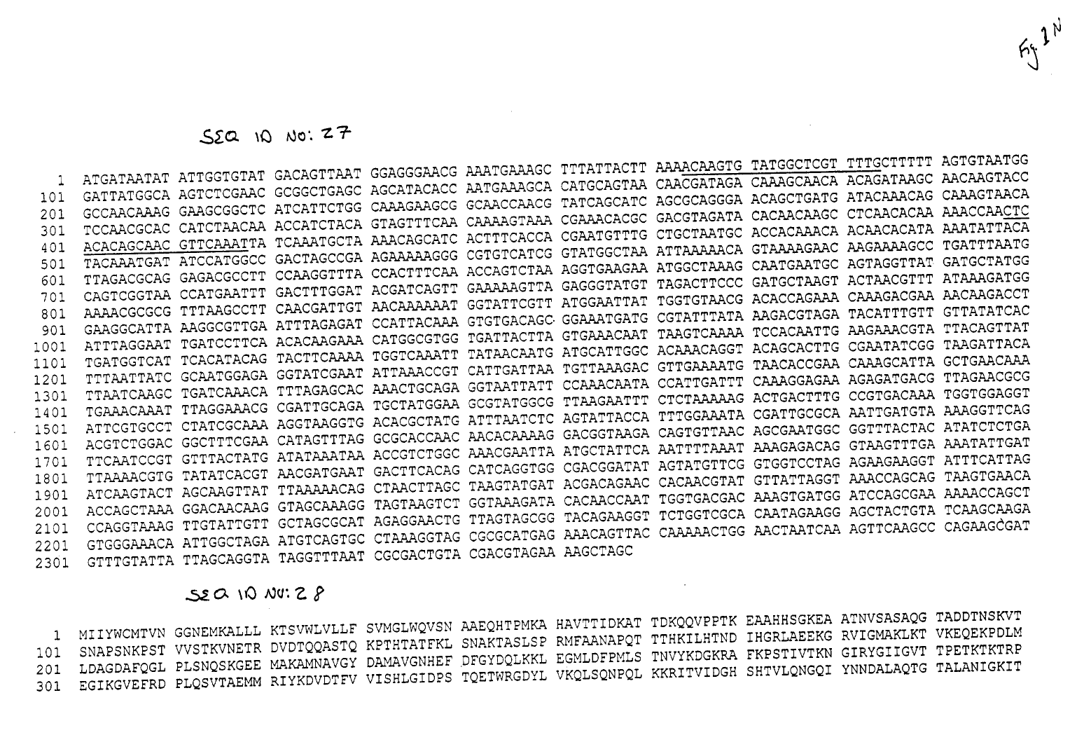

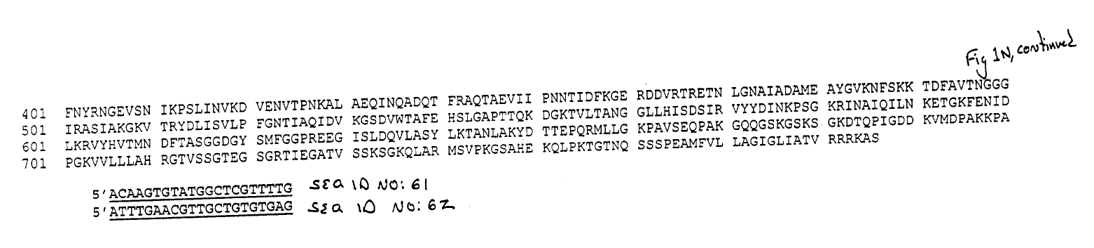

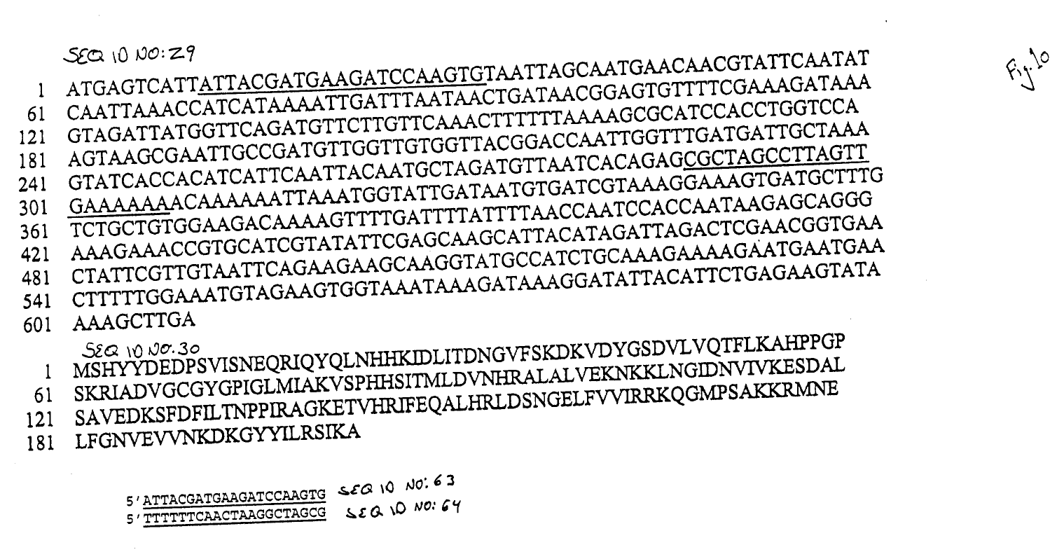

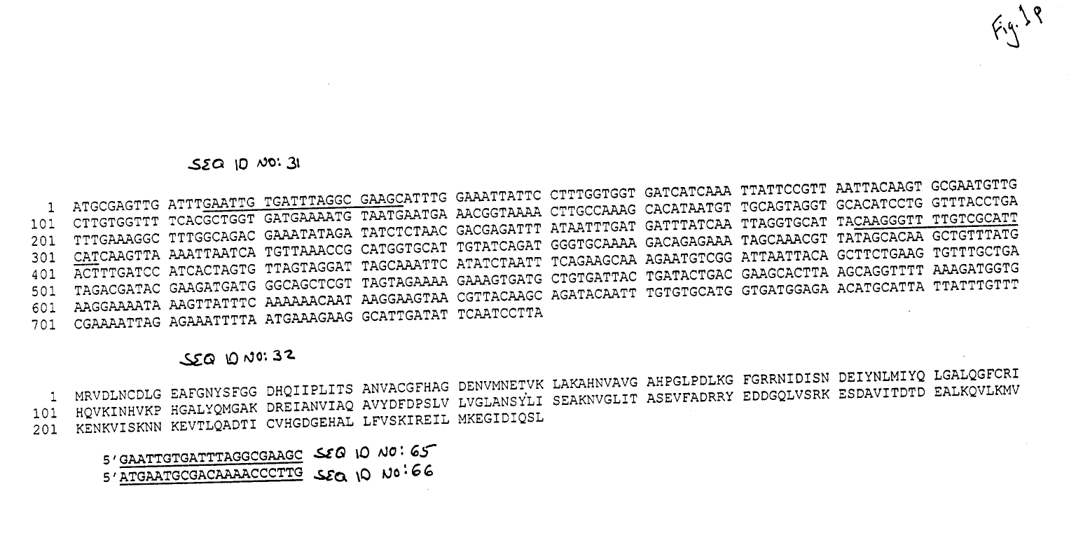

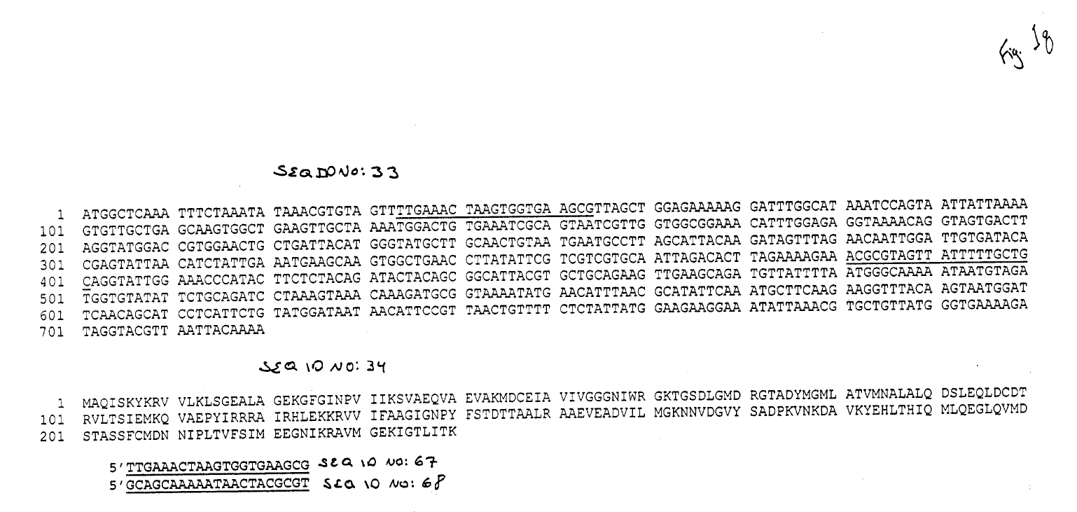

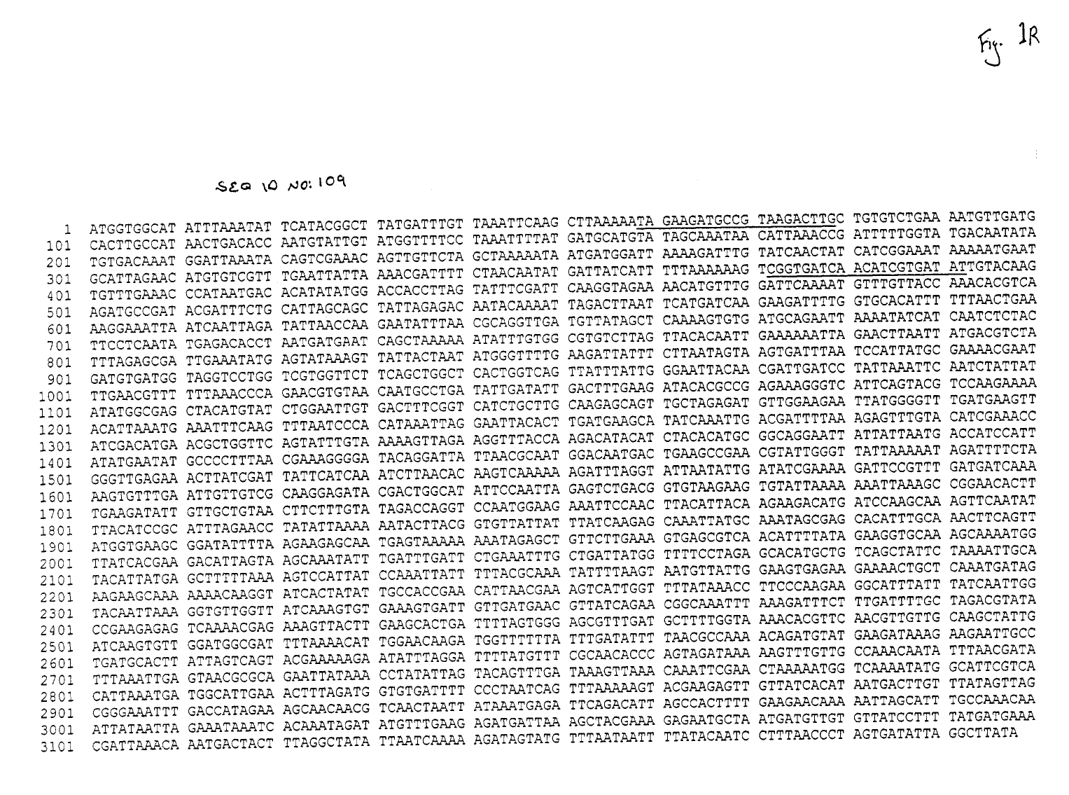

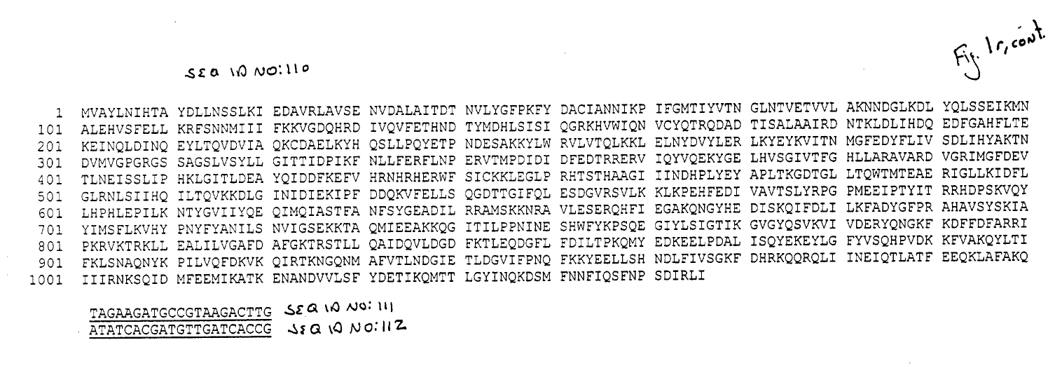

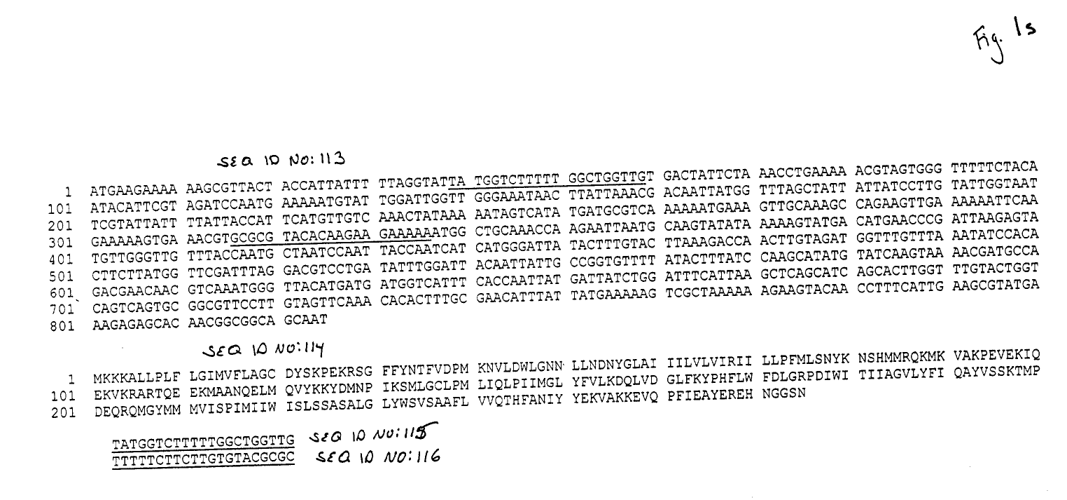

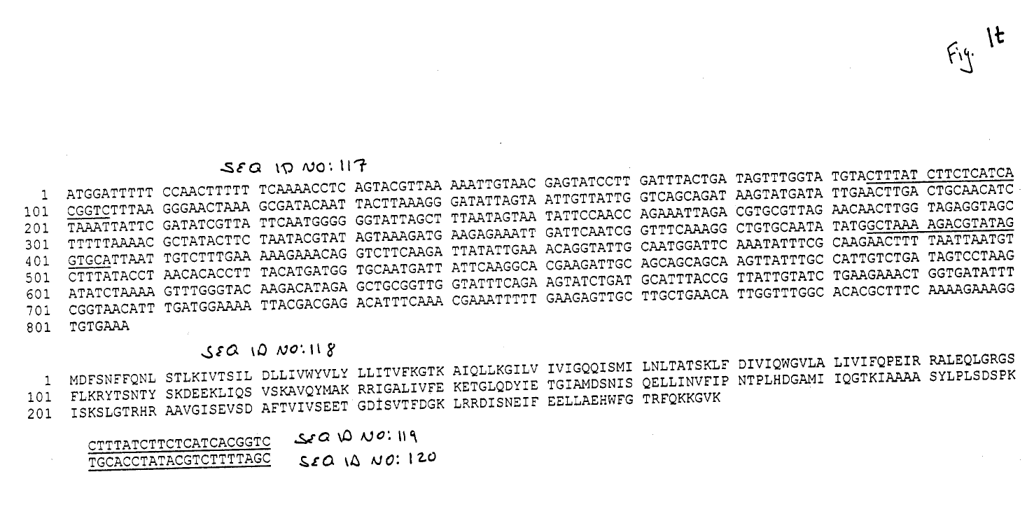

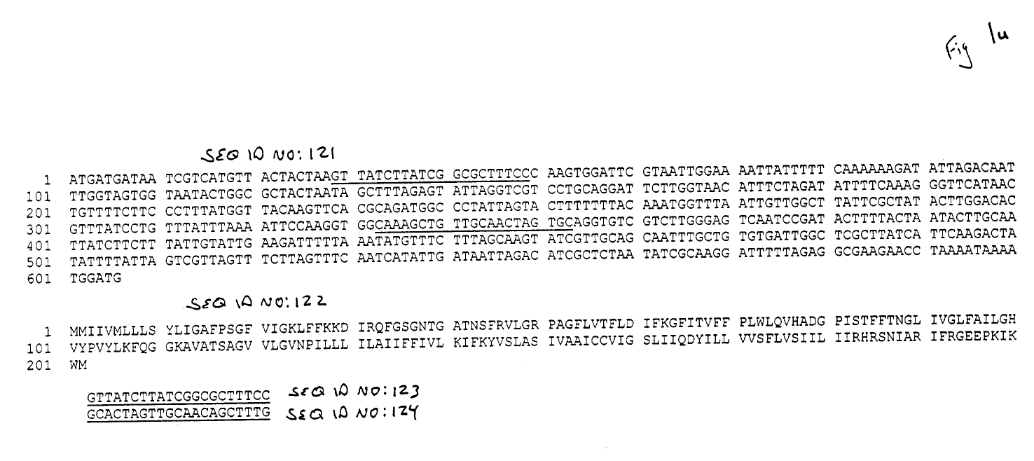

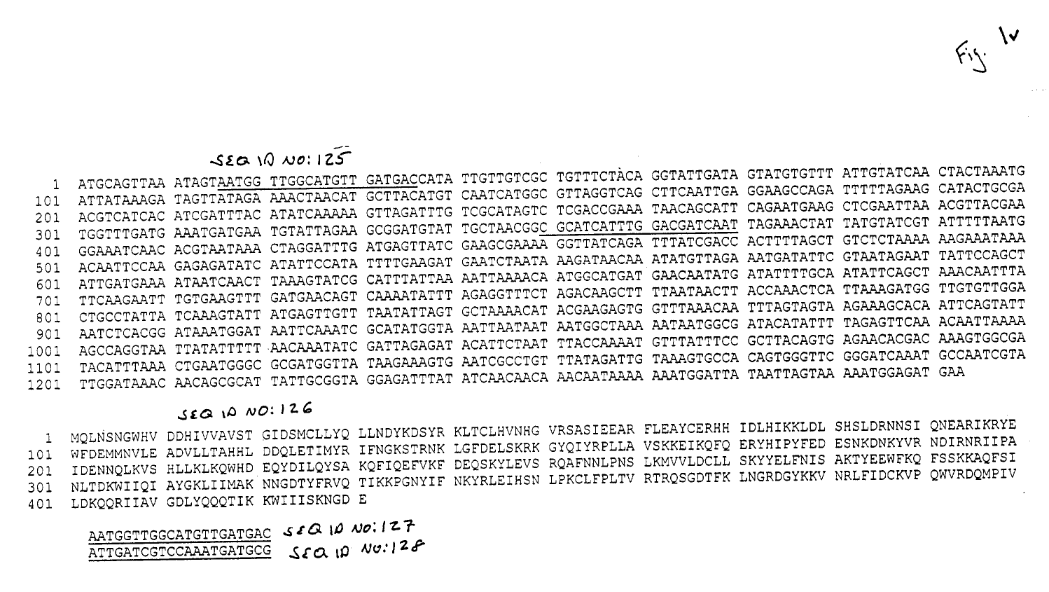

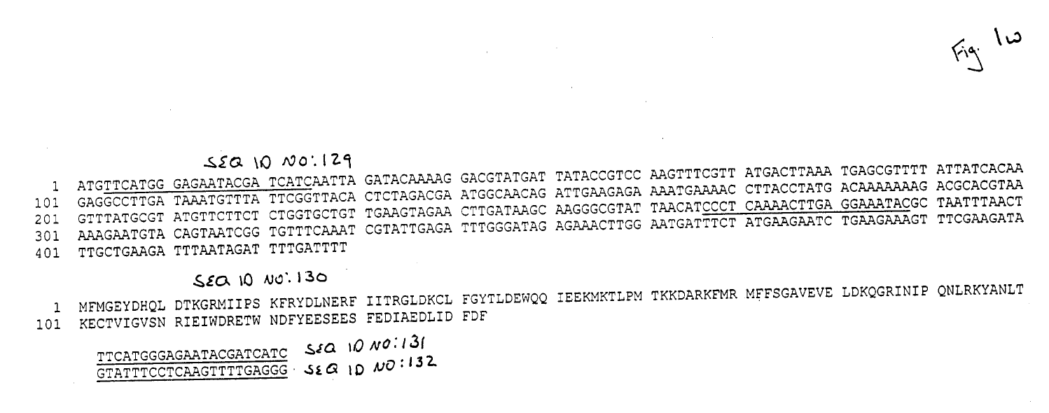

### DETAILED DESCRIPTION OF PREFERRED EMBODIMENTS OF THE INVENTION

The sequence of the S. aureus genome has been determined and includes about 3,500 coding sequences (see, for instance, Kunsch et al., EP 0 786 519 A2). As used herein, the terms “coding sequence,” “coding region,” and “open reading frame” are used interchangeably herein and refer to a nucleotide sequence that encodes a polypeptide and, when placed under the control of appropriate regulatory sequences, expresses the encoded polypeptide. The boundaries of a coding region are generally determined by a translation start codon at its 5′ end and a translation stop codon at its 3′ end. A regulatory sequence is a nucleotide sequence that regulates expression of a coding region to which it is operably linked. Nonlimiting examples of regulatory sequences include promoters, transcription initiation sites, translation start sites, translation stop sites, and terminators. “Operably linked” refers to a juxtaposition wherein the components so described are in a relationship permitting them to function in their intended manner. A regulatory sequence is “operably linked” to a coding region when it is joined in such a way that expression of the coding region is achieved under conditions compatible with the regulatory sequence.

At this time, it is not possible to predict the function of some of the polypeptides that the approximately 3,500 coding sequences of the S. aureus genome are predicted to encode. This subset of coding sequences are referred to herein as “unknown coding sequences.” Among the large number of unknown coding sequences in the S. aureus genome, those that are required for cell growth are potential novel targets for antimicrobial therapy. The function of other coding sequences of the S. aureus genome can be hypothesized by comparing an S. aureus coding sequence with a second coding sequence from another organism, where the second coding sequence has a known function. This subset of coding sequences are referred to herein as “known coding sequences.” However, even though the function of these coding sequences can be hypothesized, for many it is unknown if they are required for bacterial growth. Those known coding sequences that are required for bacterial growth are potential novel targets for antimicrobial therapy.

As used herein, a “critical coding sequence” encodes a polypeptide that is required for a bacterial cell, preferably S. epidermidis, S. saprophyticus, or S. aureus, more preferably, an S. aureus cell, to grow at a normal growth rate in vitro or in vivo, preferably in vitro. Such polypeptides are referred to herein as “critical polypeptides.” A coding sequence is a critical coding sequence when mutagenesis of the coding sequence in a bacterial cell decreases the growth rate of the bacterial cell to, in increasing levels of preference, less than about 50%, less than about 60%, less than about 80%, most preferably, less than about 90% of the growth rate of the bacterial cell that does not contain the mutated coding sequence. Methods of measuring the growth rate of microbes are well known and routine in the art. A critical coding sequence may encode a polypeptide having an unknown function, or in some aspects of the invention, encode a polypeptide having a known function. Preferably, a critical coding sequence encodes a polypeptide having an unknown function.

Preferably, a critical coding sequence is an essential coding sequence. An “essential coding sequence,” as used herein, is a coding sequence that encodes a polypeptide that is essential for the bacterial cell, preferably S. epidermidis, S. saprophyticus, or S. aureus, more preferably, an S. aureus cell, to grow in vitro or in vivo, preferably in vitro. Such polypeptides are referred to herein as “essential polypeptides.” An essential coding sequence may encode a polypeptide having an unknown function, or in some aspects of the invention, encode a polypeptide having a known function. Preferably, an essential coding sequence encodes a polypeptide having an unknown function.

Identification of these critical coding sequences, preferably essential coding sequences, provides a means for discovering new agents with different targets and mechanisms of action compared to existing agents that are used to inhibit bacteria, preferably S. epidermidis, S. saprophyticus, or S. aureus, more preferably S. aureus. The identification of essential coding sequences of microbes, preferably S. epidermidis, S. saprophyticus, or S. aureus, more preferably S. aureus, that are useful in the present invention can begin by identifying coding sequences encoding a polypeptide, preferably, a polypeptide having no known function. The coding sequences can be identified in databases, including, for instance, the S. aureus databases available from the University of Oklahoma, TIGR, NCBI, Sanger, the HGS contig database, and the HGS GSTS database. The identification of such coding sequences can include constructing contigs from data present in such databases.

As described herein, unknown coding sequences were typically identified by analyzing publicly known polynucleotide sequences. The data obtained from the database contained the nucleotide sequence of genomic clones and predicted open reading frames. However, even though the putative coding sequences may have been known, there was no indication that the coding sequences were in fact expressed, or in fact critical coding sequences. For instance, there is limited data known to the art regarding regulatory regions required for the transcription of a nucleotide sequence in S. aureus. Moreover, there is generally no evidence that the critical coding sequences and essential coding sequences identified herein are actually expressed. Thus, a person of ordinary skill, having the polynucleotide sequence of a genomic clone, would not be able to predict that an open reading frame would be transcribed, or that a coding sequence was critical, preferably, essential.

Typically, whether a coding sequence is a critical coding sequence, preferably, an essential coding sequence, can be determined by inactivating the coding sequence in a bacterial cell and determining the growth rate of the bacterial cell. Growth can be measured in vitro or in vivo, preferably in vitro. Inactivating a coding sequence is done by mutating a coding sequence present in a bacterial cell. Mutations include, for instance, a deletion mutation (i.e., the deletion of nucleotides from the coding sequence), an insertion mutation (i.e., the insertion of additional nucleotides into the coding sequence), a nonsense mutation (i.e., changing a nucleotide of a codon so the codon encodes a different amino acid), and a missense mutation (i.e., changing a nucleotide of a codon so the codon functions as a stop codon). Some insertion mutations and some deletion mutations result in frame-shift mutations. Preferably, a coding sequence in a bacterial cell is engineered to contain a deletion.

In general, an internal fragment of a selected coding sequence can be isolated or synthesized by methods known in the art, including, for instance, the polymerase chain reaction (PCR). Typically, the internal fragment is about 150 base pairs to about 350 base pairs in length, preferably about 300 base pairs. The internal fragment preferably corresponds to the 5′ end of the coding sequence. Preferably, the primers used to amplify the internal fragment contain a restriction site to allow ligation of the amplified internal fragment into a vector. For instance, when the vector is pSPT246 (described hereinbelow), one primer may contain a PstI site and the other primer may contain a SacI site.

The internal fragment is typically ligated into a vector that can be used to inactivate the coding sequence in the bacterial cell and determine if the coding sequence is a critical coding sequence or an essential coding sequence. Useful vectors include those that are unable to replicate under certain conditions in the bacterial cell that contains the coding sequence to be inactivated. Preferably, a vector is a temperature sensitive vector, i.e., it is unable to replicate in S. aureus at higher temperatures of, for instance, at least about 42° C., or a vector is a suicide vector, i.e., it is unable to replicate in S. aureus. Preferably, a temperature sensitive vector is a shuttle vector, i.e., it is able to replicate in E. coli and S. aureus under the appropriate conditions. Examples of temperature sensitive plasmids that can be used to inactivate a coding sequence in S. aureus include pSPT181 (Janzon and Arvidson, EMBO J., 9, 1391-1399 (1990)), and pSPT246 (described hereinbelow). An example of a suicide plasmid that can be used to inactivate a coding sequence in S. aureus includes pKT4 (Tegmark et al., Mol. Microbiol., 37, 398-409 (2000)).

Using these methods, the following essential coding sequences have been identified: SEQ ID NOS: 1, 3, 5, 7, 9, 11, 13, 15, 17, 19, 21, 23, 25, 27, 29, 31, 33, 109, 113, 117, 121, 125, 129, 133, and 137. The polypeptides encoded by the coding sequences are SEQ ID NOS: 2, 4, 6, 8, 10, 12, 14, 16, 18, 20, 22, 24, 26, 28, 30, 32, 34, 110, 114, 118, 122, 126, 130, 134, and 138, respectively. Of these essential coding sequences, one coding sequence (SEQ ID NO:33) encodes uridylate kinase, and is thus a known coding sequence. Prior to this invention the uridylate kinase coding sequence was not known to be essential for the growth of S. aureus. Using these methods, a critical coding sequence having the DNA sequence set forth at SEQ ID NO:141 has been identified. The polypeptide encoded by the critical coding sequence is SEQ ID NO:142.

The coding sequences of the present invention include coding sequences that are similar to the coding sequences present in SEQ ID NO:1, 3, 5, 7, 9, 11, 13, 15, 17, 19, 21, 23, 25, 27, 29, 31, 33, 109, 113, 117, 121, 125, 129, 133, 137, 141, or the complement thereof. The similarity is referred to as structural similarity and is determined by aligning the residues of the two polynucleotides (i.e., the nucleotide sequence of the candidate coding sequence and the nucleotide sequence of the coding region of SEQ ID NO:1, 3, 5, 7, 9, 11, 13, 15, 17, 19, 21, 23, 25, 27, 29, 31, 33, 109, 113, 117, 121, 125, 129, 133, 137, 141, or the complement thereof) to optimize the number of identical nucleotides along the lengths of their sequences; gaps in either or both sequences are permitted in making the alignment in order to optimize the number of shared nucleotides, although the nucleotides in each sequence must nonetheless remain in their proper order. A candidate coding region is the coding region being compared to a coding region present in SEQ ID NO:1, 3, 5, 7, 9, 11, 13, 15, 17, 19, 21, 23, 25, 27, 29, 31, 33, 109, 113, 117, 121, 125, 129, 133, 137, or 141, or the complement thereof. A candidate nucleotide sequence can be isolated from a microbe, preferably S. aureus, or can be produced using recombinant techniques, or chemically or enzymatically synthesized. Preferably, two nucleotide sequences are compared using the Blastn program of the BLAST 2 search algorithm, as described by Tatusova, et al. (FEMS Microbiol Lett 1999, 174:247-250), and available at www.ncbi.nlm.nih.gov/gorf/b12.html. Preferably, the default values for all BLAST 2 search parameters are used, including reward for match=1, penalty for mismatch=−2, open gap penalty=5, extension gap penalty=2, gap x_dropoff=50, expect=10, wordsize=11, and filter on. In the comparison of two nucleotide sequences using the BLAST search algorithm, structural similarity is referred to as “identities.” Preferably, a polynucleotide includes a nucleotide sequence having a structural similarity with the coding region of SEQ ID NO:1, 3, 5, 7, 9, 11, 13, 15, 17, 19, 21, 23, 25, 27, 29, 31, 33, 109, 113, 117, 121, 125, 129, 133, 137, 141, or the complement thereof, of, in increasing order of preference, at least about 57%, at least about 60%, at least about 70%, at least about 80%, at least about 90%, most preferably at least about 95% identity.

The present invention includes isolated polynucleotides that include a nucleotide sequence SEQ ID NO:1, 3, 5, 7, 9, 11, 13, 15, 17, 19, 21, 23, 25, 27, 29, 31, 33, 109, 113, 117, 121, 125, 129, 133, 137, 141, or the complement thereof. As used herein, an “isolated” polypeptide or polynucleotide means a polypeptide or polynucleotide that has been either removed from its natural environment, produced using recombinant techniques, or chemically or enzymatically synthesized. Preferably, a polypeptide or polynucleotide of this invention is purified, i.e., essentially free from any other polypeptides or polynucleotides and associated cellular products or other impurities. An isolated polynucleotide of the invention may include a nucleotide sequence having, in increasing order of preference, at least about 57%, at least about 60%, at least about 70%, at least about 80%, at least about 90%, most preferably at least about 95% structural similarity with a nucleotide sequence SEQ ID NO:1, 3, 5, 7, 9, 11, 13, 15, 17, 19, 21, 23, 25, 27, 29, 31, 33, 109, 113, 117, 121, 125, 129, 133, 137, 141, or the complement thereof, where the isolated polynucleotide includes a critical coding sequence, preferably, an essential coding sequence. The present invention also includes the polypeptides encoded by the coding sequences.

Another aspect of the invention includes isolated polynucleotides consisting essentially of a nucleotide sequence SEQ ID NO:1, 3, 5, 7, 9, 11, 13, 15, 17, 19, 21, 23, 25, 27, 29, 31, 33, 109, 113, 117, 121, 125, 129, 133, 137, or 141, or the complement thereof. The polynucleotide optionally further includes from zero to up to about 5,000 nucleotides upstream and/or downstream of the nucleotide sequence SEQ ID NO:1, 3, 5, 7, 9, 11, 13, 15, 17, 19, 21, 23, 25, 27, 29, 31, 33, 109, 113, 117, 121, 125, 129, 133, 137, 141, or the complement thereof. An isolated polynucleotide of the invention may consist essentially of a nucleotide sequence having, in increasing order of preference, at least about 57%, at least about 60%, at least about 70%, at least about 80%, at least about 90%, most preferably at least about 95% structural similarity with a nucleotide sequence SEQ ID NO:1, 3, 5, 7, 9, 11, 13, 15, 17, 19, 21, 23, 25, 27, 29, 31, 33, 109, 113, 117, 121, 125, 129, 133, 137, 141, or the complement thereof, where the isolated polynucleotide includes an essential coding sequence. The polynucleotide optionally further includes from zero to up to about 5,000 nucleotides upstream and/or downstream of the nucleotide sequence SEQ ID NO:1, 3, 5, 7, 9, 11, 13, 15, 17, 19, 21, 23, 25, 27, 29, 31, 33, 109, 113, 117, 121, 125, 129, 133, 137, 141, or the complement thereof. The present invention also includes the polypeptides encoded by the coding sequences.

Insertional inactivation of critical coding sequences, preferably, essential coding sequences, allows different classes of coding sequences to be identified. Examples of different classes include, for instance, coding sequences encoding proteins involved in cell surface metabolism, enzymes involved in cellular biosynthetic pathways including cell wall biosynthesis and assembly, components of the TCA cycle, proteins similar to oligopeptide transport proteins of the ATP-binding cassette (ABC) transporter superfamily, and involved in cellular regulatory and repair processes, and coding sequences affecting morphogenesis and cell division, secretion and sorting of proteins, and signal transduction systems.

The critical coding sequences, preferably, essential coding sequences may be cloned by PCR, using microbial, preferably S. epidermidis, S. saprophyticus, or S. aureus, more preferably S. aureus, genomic DNA as the template. For ease of inserting the open reading frame into expression vectors, PCR primers may be chosen so that the PCR-amplified coding sequence has a restriction enzyme site at the 5′ end preceding the initiation codon ATG, and a restriction enzyme site at the 3′ end after the termination codon TAG, TGA or TAA. If desirable, the codons in the coding sequence may be changed, without changing the amino acids, to optimize expression of a polypeptide encoded by an essential coding sequence. For instance, if an essential coding sequence is to be expressed in E. coli, the codons of the coding sequence can be changed to comply with the E. coli codon preference (see, for instance, Grosjean and Fiers, Gene, 18, 199-209 (1982), and Konigsberg et al., Proc. Natl. Acad. Sci., USA, 80, 687-691 (1983)). Optimization of codon usage may lead to an increase in the expression of the encoded polypeptide when produced in a microbe other than the microbe from which the essential coding sequence was isolated. If the polypeptide is to be produced extracellularly, either in the periplasm of, for instance, E. coli or other bacteria, or into the cell culture medium, the coding sequence may be cloned without its initiation codon and placed into an expression vector behind a signal sequence.

Proteins may be produced in prokaryotic or eukaryotic expression systems using known promoters, vectors, and hosts. Such expression systems, promoters, vectors, and hosts are known to the art. A suitable host cell may be used for expression of the polypeptide, such as E. coli, other bacteria, including Bacillus and S. aureus, yeast, including Pichia pastoris and Saccharomyces cerevisiae, insect cells, or mammalian cells, including CHO cells, using suitable vectors known in the art. Proteins may be produced directly or fused to a polypeptide, and either intracellularly or extracellularly by secretion into the periplasmic space of a bacterial cell or into the cell culture medium. Secretion of a protein typically requires a signal peptide (also known as pre-sequence); a number of signal sequences from prokaryotes and eukaryotes are known to function for the secretion of recombinant proteins. During the protein secretion process, the signal peptide is removed by signal peptidase to yield the mature protein.

The polypeptide encoded by a critical coding sequence, preferably, an essential coding sequence, may be isolated. To simplify the isolation process, a purification tag may be added either at the 5′ or 3′ end of the coding sequence. Commonly used purification tags include a stretch of six histidine residues (U.S. Pat. Nos. 5,284,933 and 5,310,663), a streptavidin-affinity tag described by Schmidt and Skerra, Protein Engineering, 6, 109-122 (1993), a FLAG peptide (Hopp et al, Biotechnology, 6, 1205-1210 (1988)), glutathione S-transferase (Smith and Johnson, Gene, 67, 31-40 (1988)), and thioredoxin (LaVallie et al., Bio/Technology, 11, 187-193 (1993)). To remove these tags, a proteolytic cleavage recognition site may be inserted at the fusion junction. Commonly used proteases are factor Xa, thrombin, and enterokinase. Preferably, a polypeptide encoded by an essential coding sequence is isolated, more preferably, purified.

The identification of critical coding sequences, preferably, essential coding sequences, renders them useful in methods of identifying new agents according to the present invention. Such methods include assaying potential agents for the ability to interfere with expression of a critical coding sequence, preferably, an essential coding sequence, thereby preventing the expression and decreasing the concentration of a polypeptide encoded by the coding sequence. Without intending to be limiting, it is anticipated that agents can act by, for instance, interacting with a critical coding sequence, preferably, an essential coding sequence, interacting with a nucleotide sequence that is adjacent to a critical coding sequence, preferably, an essential coding sequence (e.g., a promoter sequence), or inhibiting expression of a polypeptide involved in regulating expression of a critical coding sequence, preferably, an essential coding region. Agents that can be used to inhibit the expression of a critical coding sequence, preferably, an essential coding region include, for instance, the use of anti-sense polynucleotides that are complementary to the mRNA molecules transcribed from the coding sequence, and double stranded RNA (Fire et al., Nature, 391, 806-11 (1998)).

Such methods also include assaying potential agents for the ability to bind to a polypeptide encoded in whole or in part by a DNA sequence set forth in any one of SEQ ID NO:1, 3, 5, 7, 9, 11, 13, 15, 17, 19, 21, 23, 25, 27, 29, 31, 33, 109, 113, 117, 121, 125, 129, 133, 137, 141 or the complementary strand thereof. Optionally, agents that bind to such a polypeptide can be further evaluated to determine if they inhibit the function of the polypeptide to which they bind.

A polypeptide produced by a critical coding sequence, preferably, an essential coding sequence, may be used in assays including, for instance, high throughput assays, to screen for agents that inhibit the function of the polypeptide. The sources for potential agents to be screened include, for instance, chemical compound libraries, fermentation media of Streptomycetes, other bacteria and fungi, and cell extracts of plants and other vegetations. For proteins with known enzymatic activity, assays may be established based on the activity, and a large number of potential agents can be screened for ability to inhibit the activity. Such assays are referred to herein as “enzyme assays.” Enzyme assays vary depending on the enzyme, and typically are known to the art.

For proteins that interact with another protein or nucleic acid, assays can be established to measure such interaction directly, and the potential agents screened for the ability to inhibit the binding interaction (referred to herein as “binding assays”). In another aspect of the invention, assays can be established allowing the identification of agents that bind to a polypeptide encoded by an essential coding sequence (referred to herein as “ligand binding assays”).

For proteins that interact with another protein or nucleic acid, such binding interactions may be evaluated indirectly using the yeast two-hybrid system described in Fields and Song (Nature, 340, 245-246 (1989)), and Fields and Sternglanz (Trends in Genetics, 10, 286-292 (1994)). The two-hybrid system is a genetic assay for detecting interactions between two polypeptides. It can be used to identify proteins that bind to a known protein of interest, or to delineate domains or residues critical for an interaction. Variations on this methodology have been developed to clone coding sequences that encode DNA-binding proteins, to identify polypeptides that bind to a protein, and to screen for drugs. The two-hybrid system exploits the ability of a pair of interacting proteins to bring a transcription activation domain into close proximity with a DNA-binding domain that binds to an upstream activation sequence (UAS) of a reporter coding sequence, and is generally performed in yeast. The assay requires the construction of two hybrid coding sequences encoding (1) a DNA-binding domain that is fused to a protein X, and (2) an activation domain fused to a protein Y. The DNA-binding domain targets the first hybrid protein to the UAS of the reporter coding sequence; however, because most proteins lack an activation domain, this DNA-binding hybrid protein does not activate transcription of the reporter coding sequence. The second hybrid protein, which contains the activation domain, cannot by itself activate expression of the reporter because it does not bind the UAS. However, when both hybrid proteins are present, the noncovalent interaction of protein X and protein Y tethers the activation domain to the UAS, activating transcription of the reporter coding sequence. When the polypeptide encoded by, for instance, an essential coding sequence (protein X, for example) is already known to interact with another protein or nucleic acid (protein Y, for example), this binding assay can be used to detect agents that interfere with the interaction of X and Y. Expression of the reporter coding sequence is monitored as different test agents are added to the system; the presence of an inhibitory agent inhibits binding and results in lack of a reporter signal.

When the function of a polypeptide encoded by, for instance, an essential coding sequence is unknown and no ligands are known to bind the polypeptide, the yeast two-hybrid assay can also be used to identify proteins that bind to the polypeptide. In an assay to identify proteins that bind to protein X (the target protein), a large number of hybrid coding sequences, each containing a different protein Y, are produced and screened in the assay. Typically, Y is encoded by a pool of plasmids in which total cDNA or genomic DNA is ligated to the activation domain. This system is applicable to a wide variety of proteins, and it is not even necessary to know the identity or function of protein Y. The system is highly sensitive and can detect interactions not revealed by other methods; even transient interactions may trigger transcription to produce a stable mRNA that can be repeatedly translated to yield the reporter protein. When a protein is identified that binds to an essential polyeptide, the two-hybrid system can be used in a binding assay to identify agents that inhibit binding and result in lack of a reporter signal.

Ligand binding assays known to the art may be used to search for agents that bind to the target protein. Without intending to be limiting, one such screening method to identify direct binding of test ligands to a target protein is described in Bowie et al. (U.S. Pat. No. 5,585,277). This method relies on the principle that proteins generally exist as a mixture of folded and unfolded states, and continually alternate between the two states. When a test ligand binds to the folded form of a target protein (i.e., when the test ligand is a ligand of the target protein), the target protein molecule bound by the ligand remains in its folded state. Thus, the folded target protein is present to a greater extent in the presence of a test ligand which binds the target protein, than in the absence of a ligand. Binding of the ligand to the target protein can be determined by any method which distinguishes between the folded and unfolded state of the target protein. The function of the target protein need not be known in order for this assay to be performed.

Another method for identifying ligands for a target protein is described in Wieboldt et al., Anal. Chem., 69, 1683-1691 (1997). This technique screens combinatorial libraries of 20-30 agents at a time in solution phase for binding to the target protein. Agents that bind to the target protein are separated from other library components by centrifugal ultrafiltration. The specifically selected molecules that are retained on the filter are subsequently liberated from the target protein and analyzed by HPLC and pneumatically assisted electrospray (ion spray) ionization mass spectroscopy. This procedure selects library components with the greatest affinity for the target protein, and is particularly useful for small molecule libraries.

Another method allows the identification of ligands present in a sample using capillary electrophoresis(Hughes et al., U.S. Pat. No. 5,783,397). The sample and the target protein are combined and resolved. The conditions of electrophoresis results in simultaneously fractionating the components present in the sample and screening for components that bind to the target molecule. This method is particularly useful for complex samples including, for instance, extracts of plants, animals, microbes, or portions thereof and chemical libraries produced by, for instance, combinatorial chemistry.

The agents identified by the initial screens are evaluated for their effect on survival of microbes, preferably S. epidermidis, S. saprophyticus, or S. aureus, more preferably S. aureus. Agents that interfere with bacterial survival are expected to be capable of preventing the establishment of an infection or reversing the outcome of an infection once it is established. Agents may be bacteriocidal (i.e., an agent kills the microbe and prevents the replication of the microbe) or bacteriostatic (i.e., an agent reversibly prevents replication of the microbe). Preferably, the agent is bacteriocidal. Such agents will be useful to treat a subject infected with S. epidermidis, S. saprophyticus, or S. aureus, preferably S. aureus, or at risk of being infected by S. epidermidis, S. saprophyticus, or S. aureus, preferably S. aureus.

The identification of S. aureus critical coding sequences, preferably, essential coding sequences, also provides for microorganisms exhibiting reduced virulence, which are useful in vaccines. The term “vaccine” refers to a composition that, upon administration to a subject, will provide protection against S. epidermidis, S. saprophyticus, or S. aureus, preferably, S. aureus. Administration of a vaccine to a subject will produce an immunological response to the S. aureus and result in immunity. A vaccine is administered in an amount effective to result in some therapeutic benefit or effect so as to result in an immune response that inhibits or prevents an infection by S. aureus in a subject, or so as to result in the production of antibodies to an S. aureus.

Such microorganisms that can be used in a vaccine include S. epidermidis, S. saprophyticus, or S. aureus, preferably S. aureus, mutants containing a mutation in a coding sequence represented by any one of SEQ ID NO:1, 3, 5, 7, 9, 11, 13, 15, 17, 19, 21, 23, 25, 27, 29, 31, 33, 109, 113, 117, 121, 125, 129, 133, 137, 141, or a coding sequence having structural similarity thereto. Optionally, an S. epidermidis, S. saprophyticus, or S. aureus, preferably an S. aureus, includes more than one mutation. The reduced virulence of these organisms and their immunogenicity may be confirmed by administration to a subject. Animal models useful for evaluating S. aureus virulence in a variety of conditions, including for example, pneumonia, peritonitis, endophthalmitis, endocarditis, septicemia, and arthritis, are known to the art.

While it is possible for an avirulent microorganism of the invention to be administered alone, one or more of such mutant microorganisms are preferably administered in a vaccine composition containing a suitable adjuvant(s) and a pharmaceutically acceptable diluent(s) or carrier(s). The carrier(s) must be “acceptable” in the sense of being compatible with the avirulent microorganism of the invention and not deleterious to the subject to be immunized. Typically, the carriers will be water or saline which will be sterile and pyrogen free. The subject to be immunized is a subject needing protection from a disease caused by a virulent form of S. aureus.

Any adjuvant known in the art may be used in the vaccine composition, including oil-based adjuvants such as Freund's Complete Adjuvant and Freund's Incomplete Adjuvant, mycolate-based adjuvants (e.g., trehalose dimycolate), bacteria lipopolysaccharide (LPS), peptidoglycans (ie., mumins, mucopeptides, or glycoproteins such as N-Opaca, muramyl dipeptide (MDP), or MDP analogs), proteoglycans (e.g., extracted from Klebsiela spp.), streptococcal preparations (e.g., OK432), the “Iscoms” of EP 109 942, EP 180 564 and EP 231 039, aluminum hydroxide, saponin, DEAF-dextran, neutral oils (such as miglyol), vegetable oils (such as arachis oil), liposomes, the Ribi adjuvant system (see, for example GB-A-2 189 141), or adjuvants available under the trade designation BIOSTIM (e.g., 01K2) and PLURONIC polyols. Recently, an alternative adjuvant consisting of extracts of Amycolata, a bacterial genus in the order Actinomycetales, has been described in U.S. Pat. No. 4,877,612. Additionally, proprietary adjuvant mixtures are commercially available. The adjuvant used will depend, in part, on the recipient organism. The amount of adjuvant to administer will depend on the type and size of animal. Optimal dosages may be readily determined by routine methods.

The vaccine compositions optionally may include pharmaceutically acceptable (i.e., sterile and non-toxic) liquid, semisolid, or solid diluents that serve as pharmaceutical vehicles, excipients, or media. Any diluent known in the art may be used. Exemplary diluents include, but are not limited to, polyoxyethylene sorbitan monolaurate, magnesium stearate, methyl- and propylhydroxybenzoate, talc, alginates, starches, lactose, sucrose, dextrose, sorbitol, mannitol, gum acacia, calcium phosphate, mineral oil, cocoa butter, and oil of theobroma.

The vaccine compositions can be packaged in forms convenient for delivery. The compositions can be enclosed within a capsule, sachet, cachet, gelatin, paper or other container. These delivery forms are preferred when compatible with entry of the immunogenic composition into the recipient organism and, particularly, when the immunogenic composition is being delivered in unit dose form. The dosage units can be packaged, e.g., in tablets, capsules, suppositories or cachets.

The vaccine compositions may be introduced into the subject to be immunized by any conventional method including, e.g., by intravenous, intradermal, intramuscular, intramammary, intraperitoneal, or subcutaneous injection; by oral, sublingual, nasal, anal, vaginal, or transdermal delivery; or by surgical implantation, e.g., embedded under the splenic capsule or in the cornea. The treatment may consist of a single dose or a plurality of doses over a period of time.

It will be appreciated that the vaccine of the invention may be useful in the fields of human medicine and veterinary medicine. Thus, the subject to be immunized may be a human or an animal, for example, cows, sheep, pigs, horses, dogs, cats, and poultry such as chickens, turkeys, ducks and geese.

The present invention is illustrated by the following examples. It is to be understood that the particular examples, materials, amounts, and procedures are to be interpreted broadly in accordance with the scope and spirit of the invention as set forth herein.

### EXAMPLE 1

### Identification of Critical and Essential S. aureus Coding Sequences

Identification of Unknown Coding Sequences

There are about 3500 open reading frames in the HGS database of S. aureus nucleotide sequences. A Fast A homology search was conducted on these open reading frames. This homology search of those open reading frames indicated that 662 of the open reading frames were unknown coding sequences. The methods described herein typically require an open reading frame of about 300 base pairs; 492 of the 662 open reading frames were at least 300 base pairs. Of these 492, 60 had homology with unknown open reading frames from other bacterial species, 270 had no homology with any open reading frames, and 160 had homology with eukaryotic coding sequences. A homology search was also conducted between the predicted coding sequences of the Mycoplasma genitalium genome and the coding sequences of S. aureus.

The nucleotide sequences of the unknown coding sequences are shown in Table 1. Whether these coding sequences were critical or essential was determined as described herein.

TABLE 1
Primers used to amplify unknown coding sequences from S. aureus

| Nucleotide sequence of | Primer pair used to | Predicted |
|---|---|---|
| unknown coding sequence | amplify coding sequence | polypeptide |
| SEQ ID NO:1 | SEQ ID NOs:35-36 | SEQ ID NO:2 |
| SEQ ID NO:3 | SEQ ID NOs:37-38 | SEQ ID NO:4 |
| SEQ ID NO:5 | SEQ ID NOs:39-40 | SEQ ID NO:6 |
| SEQ ID NO:7 | SEQ ID NOs:41-42 | SEQ ID NO:8 |
| SEQ ID NO:9 | SEQ ID NOs:43-44 | SEQ ID NO:10 |
| SEQ ID NO:11 | SEQ ID NOs:45-46 | SEQ ID NO:12 |
| SEQ ID NO:13 | SEQ ID NOs:47-48 | SEQ ID NO:14 |
| SEQ ID NO:15 | SEQ ID NOs:49-50 | SEQ ID NO:16 |
| SEQ ID NO:17 | SEQ ID NOs:51-52 | SEQ ID NO:18 |
| SEQ ID NO:19 | SEQ ID NOs:53-54 | SEQ ID NO:20 |
| SEQ ID NO:21 | SEQ ID NOs:55-56 | SEQ ID NO:22 |
| SEQ ID NO:23 | SEQ ID NOs:57-58 | SEQ ID NO:24 |
| SEQ ID NO:25 | SEQ ID NOs:59-60 | SEQ ID NO:26 |
| SEQ ID NO:27 | SEQ ID NOs:61-62 | SEQ ID NO:28 |
| SEQ ID NO:29 | SEQ ID NOs:63-64 | SEQ ID NO:30 |
| SEQ ID NO:31 | SEQ ID NOs:65-66 | SEQ ID NO:32 |
| SEQ ID NO:33 | SEQ ID NOs:67-68 | SEQ ID NO:34 |
| SEQ ID NO:109 | SEQ ID NOs:111-112 | SEQ ID NO:110 |
| SEQ ID NO:113 | SEQ ID NOs:115-116 | SEQ ID NO:114 |
| SEQ ID NO:117 | SEQ ID NOs:119-120 | SEQ ID NO:118 |
| SEQ ID NO:121 | SEQ ID NOs:123-124 | SEQ ID NO:122 |
| SEQ ID NO:125 | SEQ ID NOs:127-128 | SEQ ID NO:126 |
| SEQ ID NO:129 | SEQ ID NOs:131-132 | SEQ ID NO:130 |
| SEQ ID NO:133 | SEQ ID NOs:135-136 | SEQ ID NO:134 |
| SEQ ID NO:137 | SEQ ID NOs:139-140 | SEQ ID NO:138 |
| SEQ ID NO:141 | SEQ ID NOs:143-144 | SEQ ID NO:142 |

Insertion Inactivation of Unknown Coding Sequences

Inactivation was achieved by integration of a plasmid in the 5′ half of the target coding sequence by homologous recombination. An internal fragment of the selected coding sequence was synthesized by PCR. The length of the amplified fragment was between about 250 base pairs to about 350 base pairs, and included the 5′ end of the coding sequence. The primers used for amplification included additional nucleotides such that a PstI restriction site was added to one end of the amplified fragment and a SacI restriction site was added to the other end of the amplified fragment. The primers are shown in Table 1. The added restriction sites allowed ligation of the amplified fragment to the temperature sensitive shuttle vector pSPT246. pSPT264 was constructed by ligating pRN8103 and pSP64-PolyA. The pRN8103 thermosensitive replication vector contains a unique EcoRI restriction site and the vector cannot replicate in E. coli. pRN8103 is described in Novick et al., (J. Mol. Biol., 192, 209-220 (1986)). The pSP64-PolyA vector, obtained from Promega Corp. (Madison, Wis.), replicates in E. coli, but not in S. aureus. pSP64-PolyA also contains a unique EcoRI restriction site. An E. coli/S. aureus shuttle vector was constructed by digesting each vector with EcoRI, ligating the two vecotrs together, and transforming the DNA into E. coli. The resulting shuttle vector was designated pSPT264.

The recombinant plasmid (i.e., pSPT246 containing an amplified fragment) was used to transform E. coli, isolated, and then transferred to S. aureus RN4220 (described in Kreiswirth et al., Nature, 305, 709-712 (1983)) by electroporation. Transformants were selected by incubation on Nutrient agar plates containing tetracycline (10 μg/ml) at the permissive temperature (30° C.). The presence of the correct plasmid was verified by PCR.

One clone with the correct plasmid was grown on Nutrient agar with tetracycline (10 μg/ml) at 32° C. overnight to allow recombination between the plasmid and the selected chromosomal allele. To select for recombinants the bacteria were then grown at the non-permissive temperature (43° C.) for 18 hours in Brain Heart Infusion (BHI) broth without tetracycline, followed by a 1:10 dilution into BHI broth containing 5 μg/ml tetracycline. The cells were incubated overnight at 43° C. The bacterial culture was then diluted, spread on Nutrient agar plates containing 5 μg/ml tetracycline and incubated at 43° C. overnight. As the plasmid cannot replicate at 43° C., only cells with the plasmid integrated into the chromosome are tetracycline resistant and form colonies. Micro-colonies that appear at the non-permissive temperature are also considered, as they may represent mutations in coding sequences that are important (e.g., critical), but not essential, for growth.

The plasmid integrates at a low frequency at other sites in the chromosome, thus tetracycline resistant clones appeared even when the target coding sequence was essential. Therefore, ten colonies from each selection at 43° C. were tested for specific integration of the plasmid into the selected target coding sequence by PCR. A primer pair consisting of one primer that binds to the vector DNA, and a second primer that binds upstream of the target coding sequence in the chromosome was used for PCR amplification. The primer pair amplifies the intervening chromosomal-vector region, and an amplified DNA fragment is produced only if the vector integrated at the predicted location. The absence of a band suggests the vector cannot integrate, and that the coding sequence is essential. Typically, all or none out of the tested colonies were specific recombinants. In those cases where no recombinants are found the target coding sequence is considered essential. For a number of target coding sequences (both essential and non-essential) the same results have been obtained when the whole selection procedure was repeated.

This protocol has successfully been used to analyze about 300 out of the of 492 unknown complete or partial coding sequences identified. Out of the analyzed coding sequences, 26 appeared to be critical and were further analyzed as described below.

### EXAMPLE 2

### Cloning of Essential S. aureus Coding Sequences and Expression in E. coli

Overview of the Expression System and Cloning Procedure

The overexpression of S. aureus proteins is accomplished using the Qiagen Type ATG expression system (Qiagen Gmbh, Santa Clara, Calif.). This system utilizes E. coli strain “M15” whose genotype has been described by Qiagen as Nal^{S}, Str^{S}, rif^{S}, lac⁻, ara⁻, gal⁻, mtl⁻, F⁻, recA⁺, uvr⁺. Two replication compatible vectors, pREP4 and pQE-60 (each obtained from Qiagen), are introduced into the M15 strain during the procedure. Alternatively, pQE-70 can be used instead of pQE-60. The pREP4 vector is a pACYC-derived vector that contains the lacI gene encoding for the Lactose (LacI) repressor protein, and the vector encodes kanamycin drug resistance. The expression vector pQE-60 is a pBR322-derived vector that contains a modified T5 phage promoter, a strong ribosme binding site (RBS), and the coding sequence of the specific S. aureus coding sequence to be expressed. The T5 promoter modifications include the placement of operator sites for binding and regulation of the promoter by the LacI repressor. Induction of expression is performed by the addition of IPTG (isopropylthio-β-D-galactoside) to a log phase culture.

The general cloning strategy is to first amplify the specific coding sequence from S. aureus genomic DNA using PCR primers to the 5′ and 3′ ends of the coding sequence sequence. The PCR primers are designed to add an NcoI and a BglII restriction site at the 5′ and 3′ ends of the coding sequence respectively. The coding sequence should be free of any NcoI or BglII restriction sites. If such sites are present, they are eliminated using site-directed PCR mutagenesis procedures known to the art. Alternatively, a different restriction site, for instance a BamHI restriction site, is used instead of a BglII restriction site. The amplified S. aureus coding sequence is ligated into pCR-2.1 (Invitrogen, Carlsbad, Calif.) and transformed into E. coli using techniques known to the art. Colonies are screened for the presence of the coding sequence by PCR amplification or vector restriction analysis. Clones are randomly selected and the nucleotide sequence of the insert DNA, i.e., the S. aureus coding sequence, is determined to confirm authenticity of the insert.

The pCR-2.1 vector containing the desired coding sequence is digested with NcoI/BglII and the coding sequence iss isolated and ligated into the corresponding NcoI/BglII restriction sites of pQE-60. The ligation mixture is used to electroporate the vector DNA into the M15 strain that contained the pREP4 vector. The resulting transformants are screened by PCR or restriction analysis. Candidates are grown in a shake-flask and screened for the overexpression of a protein band having the appropriate size as analyzed by SDS-PAGE or Western analysis. Anti-His antibody (Invitrogen) is used in the Western analysis. A single candidate is selected for the overexpresion and isolation of the protein encoded by each coding sequence.

Culture and Media

The medium for cloning and maintenance of cells containing recombinant plasmids in E. coli is LB supplemented with the appropriate antibiotic (100 μg/ml ampicillin, 25 μg/ml kanamycin). S. aureus was grown in Mueller-Hinton medium. Competent INVF′α cells (Invitrogen, Carlsbad, Calif.) are used according to the manufacturer's direction. The M15 pREP-4 strain was purchased from Qiagen. SOC medium was used in the electroporation of cells. LB and SOC media are described in Sambrook et al. (Molecular Cloning: A Laboratory Manual., Cold Spring Harbor Laboratory Press, pp. A1-A4 (1989)). Mueller-Hinton medium is described in Atlas et al., Handbook of Microbiological Media, CRC Press.

Design of the pQE60 Expression Vector

The portion of the pQE-60 DNA sequence containing the T5 promoter, the RBS, the ATG start codon (in bold), the NcoI restriction site (underlined), the BglII restriction site (underlined), 6 His tag (double underline), and the TAA stop codon (in bold) is shown (SEQ ID NO:89):

CTCGAGAAAT CATAAAAAAT TTATTTGCTT
TGTGAGCGGA TAACAATTAT AATAGATTCA
ATTGTGAGCG GATAACAATT TCACACAGAA
TTCATTAAAG AGGACAAATT AACCATGGGA
GGATCCAGAT CTCATCACCA TCACCATCAC
TAAGCTTAAT TA NcoI
BglII

The S. aureus coding sequences are modified by PCR to contain compatible in-frame NcoI and BglII restriction sites.

Primer Design

The general formula for the design of the primer to the 5′ portion of the S. aureus coding sequence is usually 5′-CCATGGGAN₂₀₋₃₀, and the general formula for the 3′ primer is usually 5′-AGATCTN₂₀₋₃₀. These primers add the NcoI and BglII restriction sequences. The first “N” nucleotide of the 5′ sequence correspond to the codon of the second amino acid of the S. aureus coding sequence after its ATG start The first “N” nucleotide of the 3′ primer corresponds to the third nucleotide in the codon preceding the stop codon of the S. aureus coding sequence. The number of nucleotides to include in the primer varied depending on the specific DNA sequence, but is typically in a range of 20 to 30 bases. The primers are phosphorylated. Examples of primers that can be used to amplify some coding sequences are shown in Table 2.

TABLE 2
Primers used to amplify essential coding sequences from S. aureus

| | | Resulting | Predicted poly- |
|---|---|---|---|
| Essential | Primer pair used to | sequence in | peptide and pre- |
| coding | clone coding | pQE-60 or | dicted molecular |
| sequence | sequence | pQE-70 | weight |
| SEQ ID NO:1 | SEQ ID NOs:91-92 | SEQ ID NO:69 | SEQ ID NO:70, |
| | | | 28.1 kD |
| SEQ ID NO:3 | SEQ ID NOs:93-94 | SEQ ID NO:71 | SEQ ID NO:72, |
| | | | 31.6 kD |
| SEQ ID NO:5 | SEQ ID NOs:95-96 | SEQ ID NO:73 | SEQ ID NO:74, |
| | | | 28.5 kD |
| SEQ ID NO:9 | SEQ ID NOs:97-98 | SEQ ID NO:75 | SEQ ID NO:76, |
| | | | 37.4 kD |
| SEQ ID NO:11 | SEQ ID NOs:99- | SEQ ID NO:77 | SEQ ID NO:78, |
| | 100 | | 40.9 kD |
| SEQ ID NO:13 | SEQ ID NOs:101- | SEQ ID NO:79 | SEQ ID NO:80, |
| | 102 | | 30.4 kD |
| SEQ ID NO:15 | SEQ ID NOs:103- | SEQ ID NO:81 | SEQ ID NO:82, |
| | 104 | | 78.1 kD |
| SEQ ID NO:17 | SEQ ID NOs:105- | SEQ ID NO:83 | SEQ ID NO:84, |
| | 106 | | 57.4 kD |
| SEQ ID NO:19 | SEQ ID NOs:107- | SEQ ID NO:85 | SEQ ID NO:86, |
| | 108 | | 16.1 kD |

Preparation of the S. aureus Genomic DNA

Strain ISP3 (obtained from S. Arvidson, Karolinska Institute) is used to inoculate 10 mls of Mueller-Hinton broth. After overnight growth at 37° C., 1.5 mls of culture are pelleted in an eppendorf tube and then resuspended in 400 μl of TE, pH 8.0 (Sambrook et al. (Molecular Cloning: A Laboratory Manual., Cold Spring Harbor Laboratory Press, p. B.20 (1989)). Following the addition of 50 μl lysostaphin solution (10 mg/ml), the cells are incubated at 37° C. for 1 hour. Seventy microliters of 10% SDS and 10 μl of proteinase K (20 mg/ml) are added and the incubation continued at 37° C. for another hour. After the addition of 100 μl of 5 M NaCl, the cell suspension is vortexed and 80 μl of a solution containing 10% hexadecyltrimethyl ammonium bromide, 0.7 M NaCl (CTAB/NaCl) is added. The cells are vortexed and then incubated at 65° C. for 10 minutes. Following the addition of an equal volume of 25:24:1 phenol:chloroform:isoamyl alcohol, the cells are vortexed and centrifuged for 5 minutes. The aqueous phase is then transferred to a fresh tube, leaving behind the white CTAB/NaCl interface. The extraction is repeated, and the aqueous layer is again transferred to a fresh tube. Following the addition of an equal volume of isopropanol, the tube is gently mixed causing a stringy precipitate to form. A Pasteur pipette fashioned into a small hook is used to gently remove the precipitate and to transfer it into another tube containing 1 ml of 70% ethanol. The tube is centrifuged, and the resulting pellet is washed once with 70% ethanol. After drying, the DNA pellet is resuspended in 100 μl of water and the concentration of the recovered DNA is determined using techniques known in the art.

PCR Amplification

PCR reactions are performed using either the Perkin-Elmer Cetus GeneAmp 9600 or 2400 thermal cyclers (Perkin-Elmer, Norwalk, Conn.). The deoxynucleotide mix and the Pfu DNA polymerase are purchased from Stratagene (La Jolla, Calif.). The AmpliTaq Gold kit is purchased from Perkin Elmer. The PCR synthesis protocol for long template amplification is as follows: 1 μg of S. aureus genomic DNA, 10 μl of 10× reaction buffer (with 15 mM MgCl₂), 500 ng of each primer, 16 μl of 1.25 mM dNTP's, 1 μl of AmpliTaq Gold, and water to 100 μl are added per PCR microtube. The DNA is amplified for 35 cycles using Cycle Program of 95° C. for 5 minutes followed by 35 cycles of 94° C. for 30 seconds, 50° C. for 1 minute and 72° C. for 3 minutes, an extension at 72° C. for 5 minutes, and finally 40° C. on hold. A 10 μl aliquot of the synthesis reaction is loaded onto a 1.2% agarose gel to confirm the presence and size of the synthesized fragment. The PCR product is produced by combining multiple PCR reaction, EtOH precipitating the DNA, and cutting the desired fragment out of a 1.2% agarose gel. The DNA is isolated from the agarose using Amicon Ultrafree-DA extraction filters (Millipore Corp., Bedford, Mass.). The filters are used according to manufacturer's directions.

Ligation and Transformation

The pQE-60 vector and the pCR2.1 vector containing the S. aureus coding sequence are digested with NcoI and BglII restriction enzymes. The pQE-60 vector fragment and the S. aureus coding sequence are isolated from an agarose gel. The two DNAs are ligated and transformed into electrocompetent M15 cells containing pREP-4, and plated on LB agar with ampicillin and kanamycin supplementation. Ligase is purchase from BioLab (Beverley, Mass.), and used in accordance with the manufacturer's instructions. Electroporation of the ligated DNA into M15 pREP-4 cells is performed using a Bio-Rad Gene Pulser (Hercules, Calif.). Competent cells are prepared from 1 liter of cells with an optical density of 1 at A₅₅₀. The cells are chilled and washed successively with 1 liter and 0.5 liters of ice cold sterile water. The cells are resuspended in 20 mls of ice cold sterile 10% glycerol, re-centrifuged and placed into a final suspension of 2 to 3 mls of cold sterile 10% glycerol. Fifty microliters of cells are mixed with 5 μls or less of ligated DNA. The cell/DNA mixture is transferred to an electroporation cuvette and pulsed with the settings at 25 μF, 2.5 kV, and the Pulse Controller set to 200 Ω. One ml of SOC media is then added. The cells are incubated at 30° C. for one hour and plated on selective media.

Several resultant colonies from the transformation are selected at random and vector DNA is isolated using the Miniprep or Maxiprep kits purchased from Qiagen. The vector DNA is isolated according to the manufacturer's instructions. The candidates are screened by restriction enzyme digestions. Restriction enzymes are purchased from New England BioLab (Beverly, Mass.). Restriction enzymes are used according to the manufacturer's instructions.

Expression Conditions

The expression culture is streaked on an LB plate containing ampicillin and kanamycin. A single colony isolate is used to inoculate 50 mls of LB medium supplemented with ampicillin and kanamycin and grown overnight at the desired temperature. Following sub-culture into the suitable volume of the identical media at 0.50 A₅₅₀/ml, the culture is grown at the same temperature with vigorous aeration until an A₅₅₀ of 3.0 was reached. The culture is induced by the addition of IPTG to a final concentration of 1 mM. Culture aliquots are removed at 0, 2, and 4 hours post-induction for SDS-PAGE or Western analysis. Cells are harvested for protein isolation between 4 and 6 hours. Proteins are isolated using a metal-chelate affinity chromatography purification system (QIAEXPRESS, Qiagen).

### EXAMPLE 3

### Use of Essential Coding Sequence Products in Screen for Antimicrobial Agents

Individual purified proteins (i.e., target proteins) are combined with samples and screened for ligands that would bind the target protein. The method used to screen is described in Hughes et al., U.S. Pat. No. 5,783,397. The screening is conducted by Cetek Corporation, Marlborough, Mass.

### EXAMPLE 4

### Cloning of S. aureus Uridylate Kinase Coding Sequence for Expression in E. coli

The S. aureus pyrH coding sequence encoding for uridylate kinase was cloned into the expression vector pQE60 for production of recombinant uridylate kinase in the E. coli strain M15 containing the plasmid pREP4. Cloning of the pyrH coding sequence was by PCR with two oligonucleotide primers 5′ CCCGGGCCATGGCTCAAATT (SEQ ID NO:90) and 5′ GGGCCCAAGCTTAGTGATGG (SEQ ID NO:145), using S. aureus genomic DNA as the template. The PCR product was treated with restriction enzymes NcoI and HindIII, purified by agarose gel electrophoresis, and ligated into pQE60 disgested with NcoI and HindIII. The ligation mixture was transformed into M15 cells containing pREP4; transformants were selected and the nucleotide sequence of the pyrH coding sequence was verified by restriction enzyme analysis and DNA sequencing. The resulting plasmid for production of S. aureus uridylate kinase in E. coli was designated pQE60-UMK. Procedures for DNA and plasmid preparation, restriction enzyme treatment, ligation, and transformation were according to those described in Sambrook et al. (Molecular Cloning: A Laboratory Manual., Cold Spring Harbor Laboratory Press (1989)).

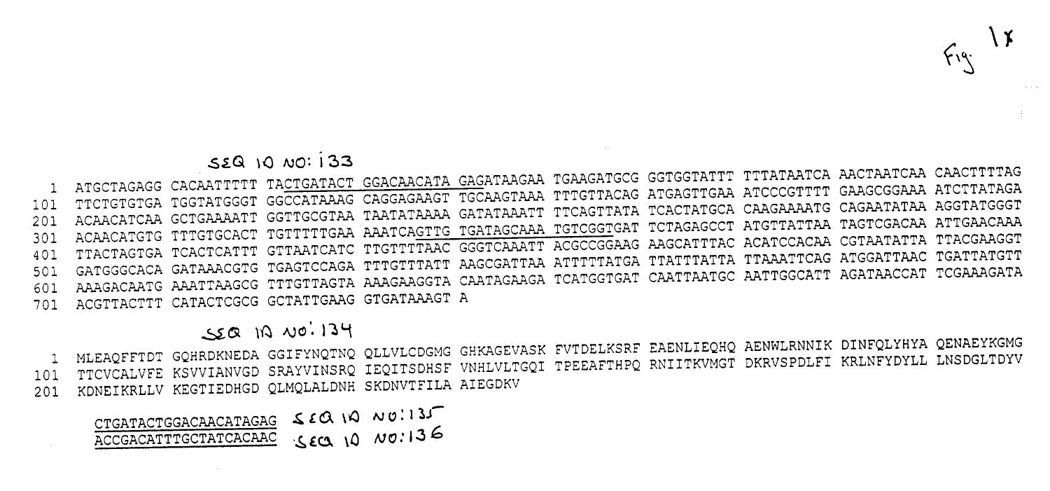

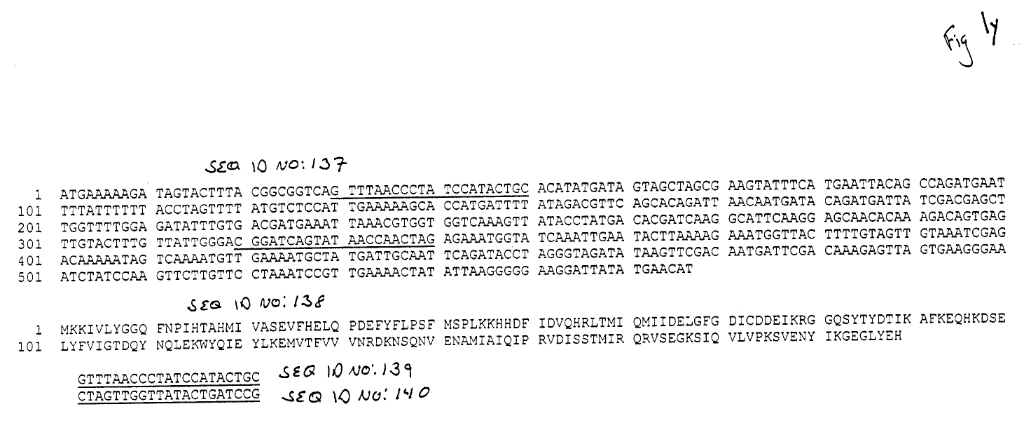

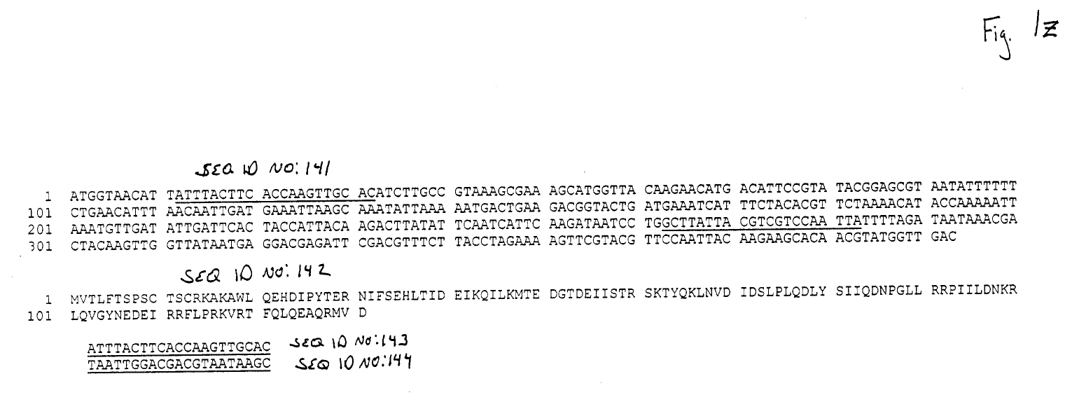

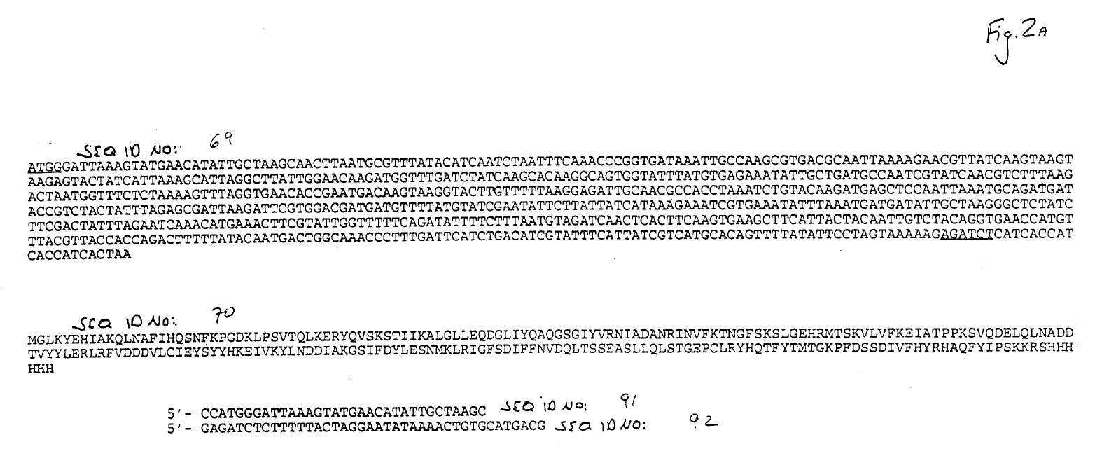

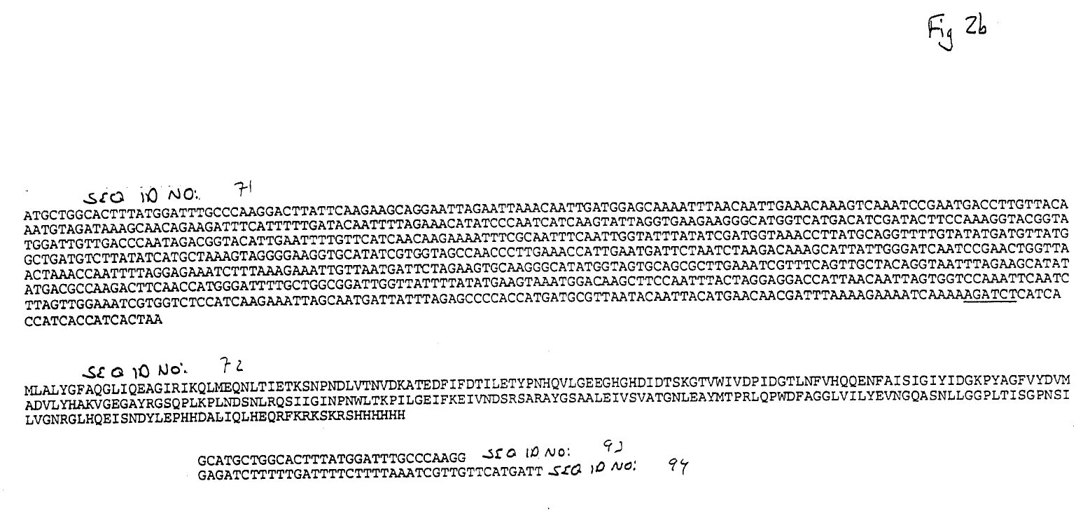

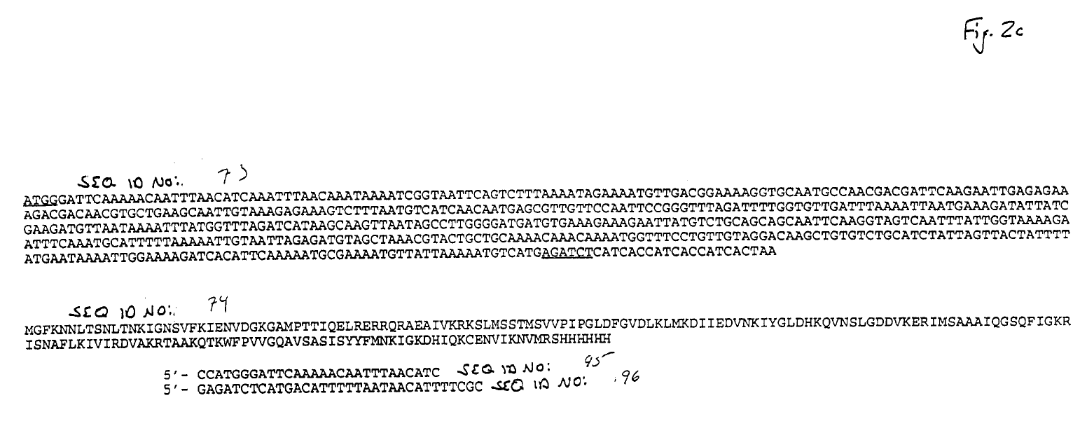

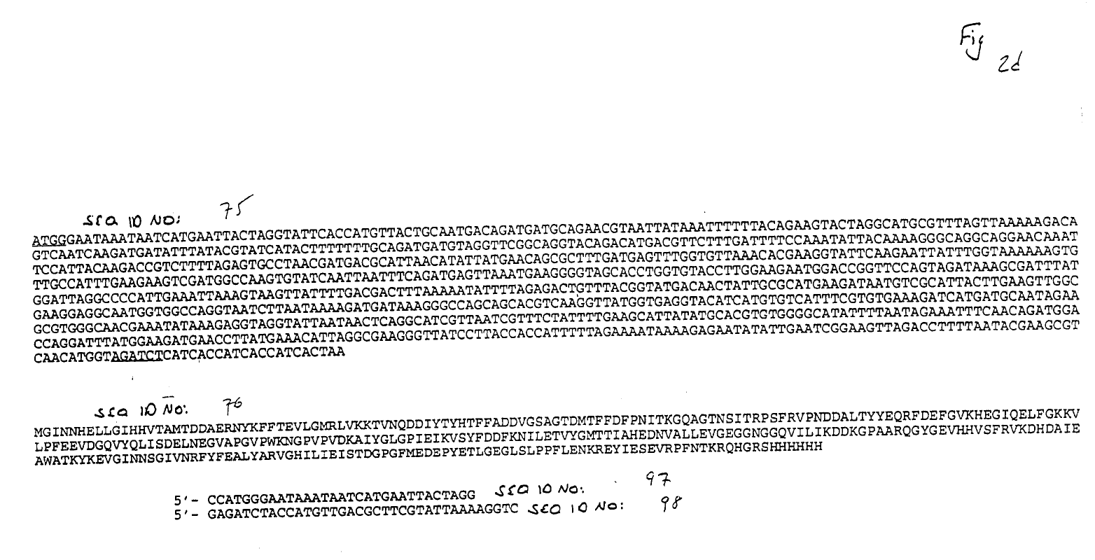

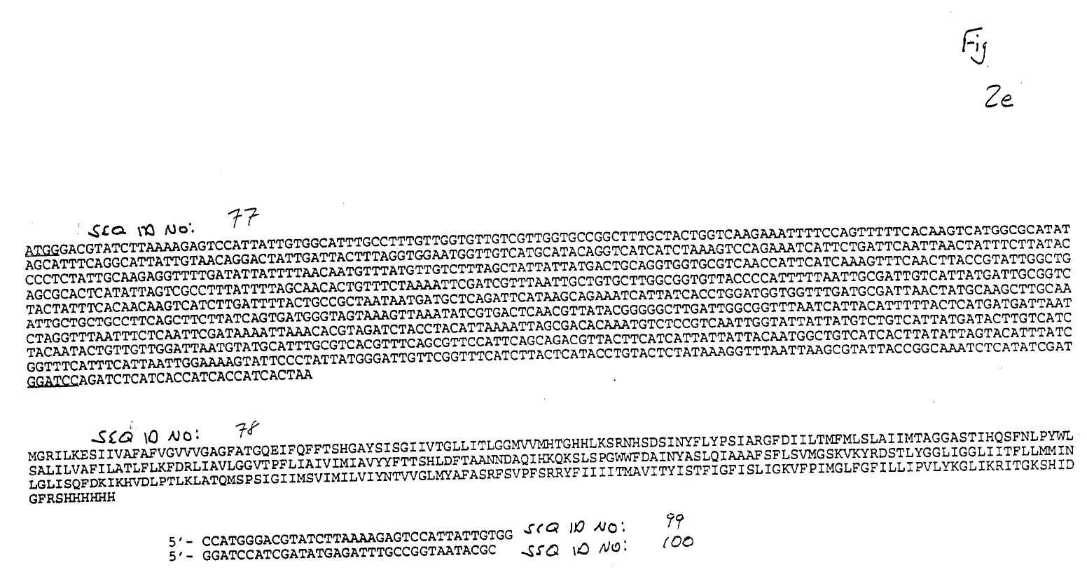

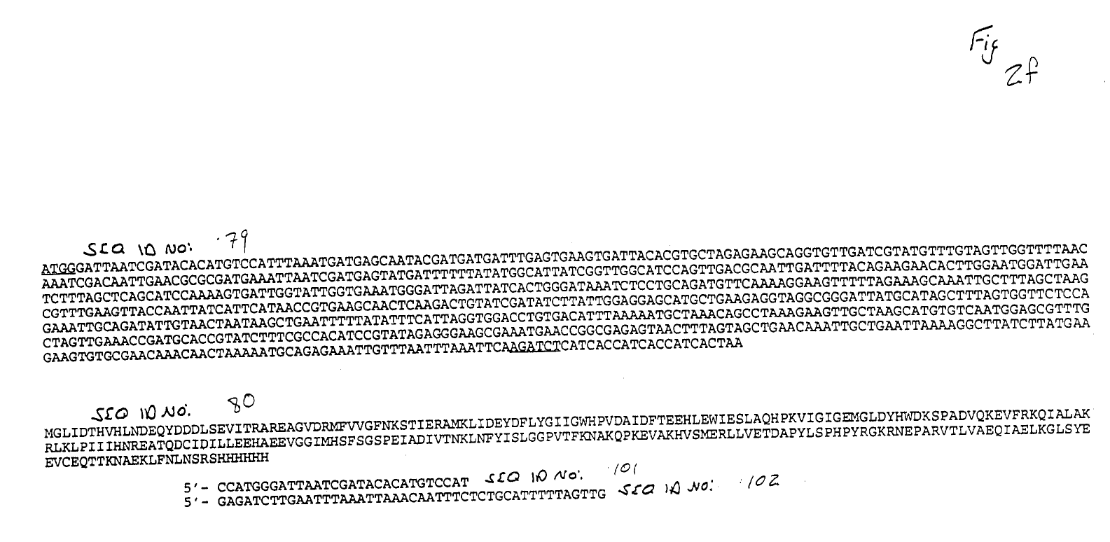

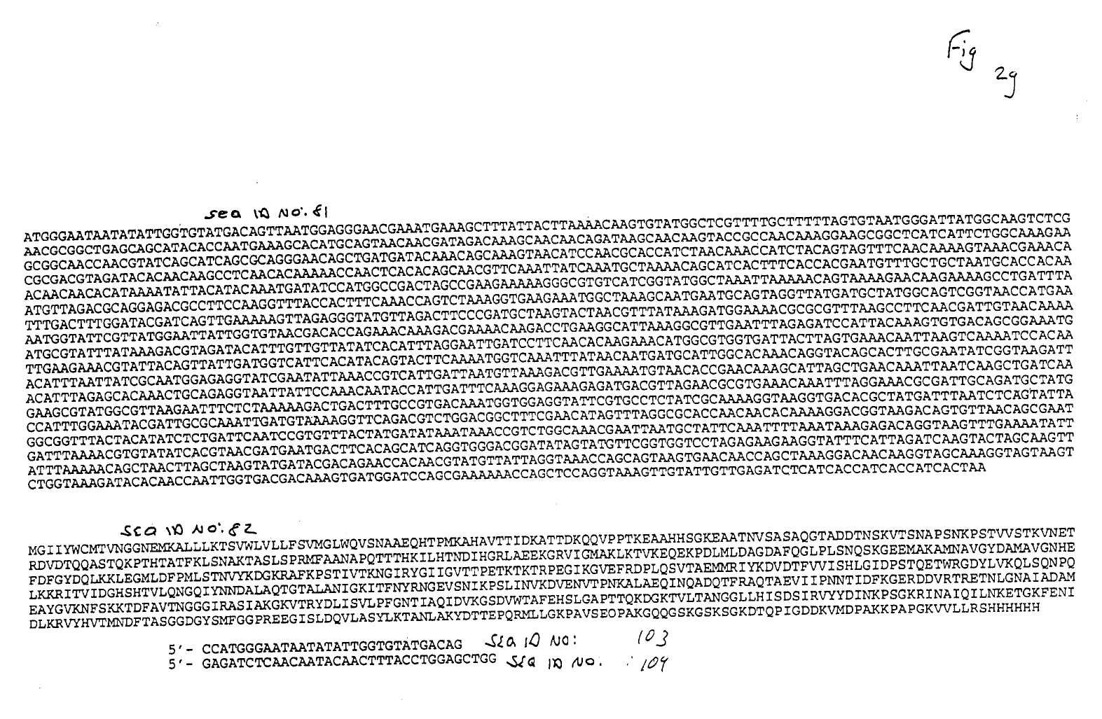

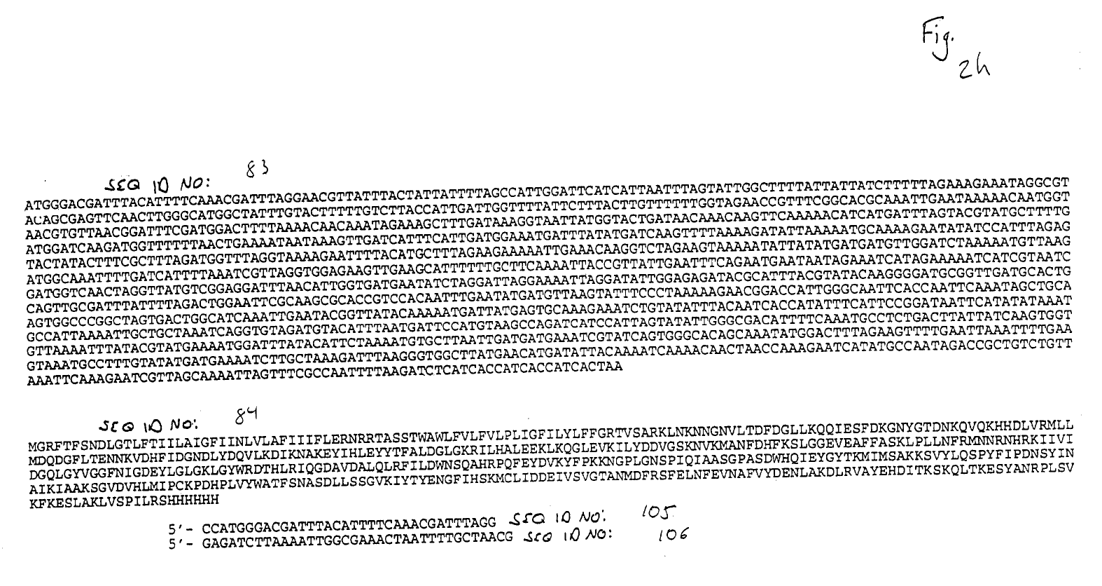

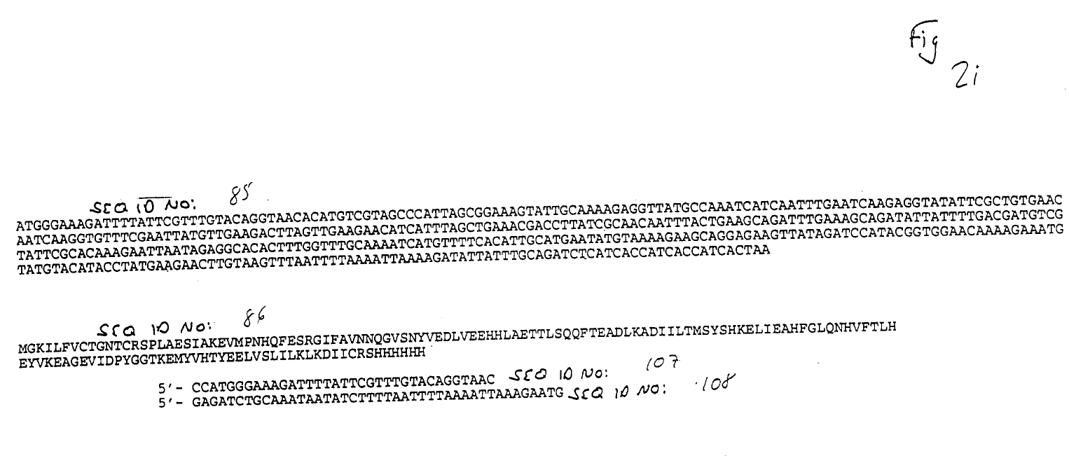

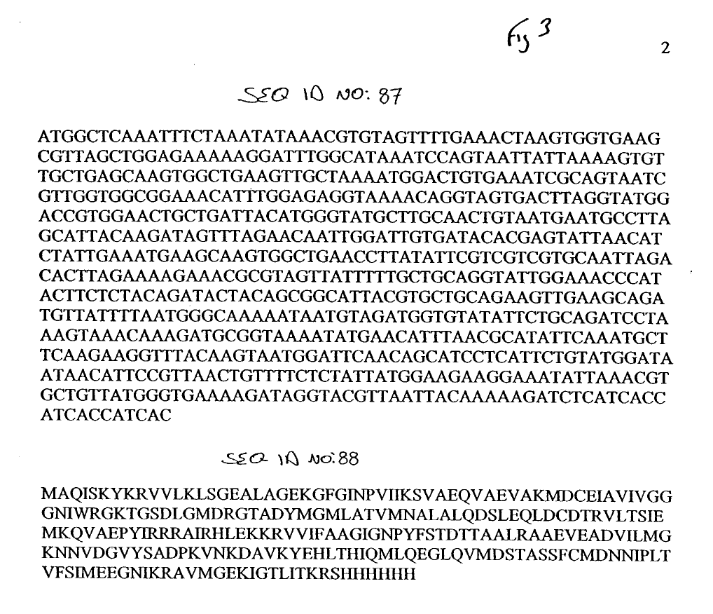

The nucleotide and amino acid sequences of the recombinant uridylate kinase are shown in FIG. 3. Six histidine residues are added to the C-terminus for purification with Ni—chelating chromatography. Due to the use of NcoI site for cloning and vector sequence, a alanine residue is added after the initiation methionine, and arginine and serine residues are added prior to the histidine residues.

### EXAMPLE 5

### Production of Recombinant S. aureus Uridylate Kinase

The production strain, designated M15(pQE60-UMK), was grown to A₅₅₀ of about 1 at 30° C. in NS86 medium. NS86 medium is 2.6 grams (g) K₂HPO₄, 10.9 g NaNH₄HPO₄-4H₂O, 2.1 g citric acid, 0.67 g (NH₄)₂SO₄, 0.25 g MgSO₄-7H₂O, 10.4 g yeast extract, and 5 g glycerol in 1 liter of H₂O. The NS86 medium was supplemented with 100 μg/ml ampicillin and 25 μg/ml kanamycin. Frozen ampules with 20% glycerol added as a cryoprotectant were prepared and stored in liquid nitrogen.

Seed culture was prepared by inoculation of 0.1 ml thawed cells from an ampule into 50 ml of NS86 medium, grown overnight at 30° C., and used to inoculate 100 ml MIM medium. MIM medium is 32 g tryptone, 20 g yeast extract, 6 g Na₂HPO₄, 3 g KH₂PO₄, 0.5 g NaCl, and 1 g NH₄Cl in 1 liter of H₂O containing 100 μg/ml ampicillin and 25 μg/ml kanamycin to A₅₅₀ 0.1. Cells were grown at 30° C. overnight to A₅₅₀ 7-8 and used for 10-liter fermentation.

Seed culture was used to inoculate a 10-liter fermentor (New Brunswick Microgens) with MIM medium to A₅₅₀ 0 1. When cells grew up to a density of A₅₅₀ of about 1 at 30° C., isopropyl-β-D-thiogalactoside was added to 1 mM to induce the expression of the recombinant protein. Cells were harvested at 2.5 hours post-induction and stored frozen. The average amount of uridylate kinase produced from a 10-liter fermentation was estimated at 170 mg/l and corresponded to about 20-25% of total cell protein.

### EXAMPLE 6

### Purification of Recombinant S. aureus Uridylate Kinase

The frozen cells from a 10-liter fermentation were thawed and mixed with 200 ml cold lysis buffer (50 mM Tris, pH 7.8 at 22° C., 500 mM NaCl, 10% glycerol, 25 mM imidazole, 5 mM 2-mercaptoethanol, 0.1 mg/ml DNase). The pellet was homogenized to yield a uniform suspension, and processed two times through a Rainie homogenizer to lyze the cells. The lysed cells were centrifuged at 35,000×g for 75 minutes and the supernatant liquid was filtered sequentially through Nalgene 0.45 micron and 0.2 micron filters to remove particulates prior to column chromatography.

Column chromatography was carried out at 4° C., using a 2.6 cm×6.7 cm column packed with Qiagen Ni-NTA Superflow resin. The column was washed with 3 bed volumes of water and equilibrated with 3 bed volumes of equilibration buffer (50 mM Tris, pH 7.8 at 22° C., 500 mM NaCl, 10% glycerol, 5 mM 2-mercaptoethanol) containing 25 mM imidazole. The filtered supernatant was applied to the column at a rate of 3 bed volumes per hour. After loading, the column was washed with equilibration buffer with 25 mM imidazole until the absorbance at 280 nanometers (nm) decreased to 50% of baseline, followed by 6-7 bed volumes of equilibration buffer plus 40 mM imidazole and then 6-7 bed volumes of equilibration buffer plus 50 mM imidazole. The bound uridylate kinase was eluted at a rate of 2-3 bed volumes per hour with equilibration buffer plus 300 mM imidazole, and was recovered in four separate fractions which were pooled, diluted 3-fold to reduce the protein concentration, and dialyzed against 50 mM Tris, pH 7.8 at 22° C., 500 mM NaCl, 10% glycerol, 5 mM 2-mercaptoethanol. The dialyzed pool was stored frozen until further use.

This isolation yielded about 700 mg of recombinant uridylate kinase protein at a purity of 95-98%. N-terminal sequencing showed that the N-terminal methionine was absent, which is expected due to the activity of host methionine aminopeptidase.

### EXAMPLE 7

### Enzyme Assays for Uridylate Kinase Activity

Uridylate kinase catalyzes the transfer of a phosphoryl group from ATP to UMP to form UDP. In the cell, UDP is the substrate/precursor in several metabolic pathways including RNA and synthesis. A spectrophotometry assay was established by coupling the uridylate kinase reaction to NADH oxidation using pyruvate kinase and lactate dehydrogenase, which ultimately convert the products of uridylate kinase reaction to lactate and NAD⁺. NADH oxidation was monitored by following the decrease in absorbance at 340 nm. EDTA at a final concentration of 5 mM can be used to stop the assay. This assay was optimized in high-throughput format in 96-well microtiter plates to screen for agents that inhibit uridylate kinase. A secondary assay for pyruvate kinase and lactate dehydrogenase coupling enzymes was also developed to test the specificity of agents detected in the primary coupled assay.

The coupled assay is diagramed below.

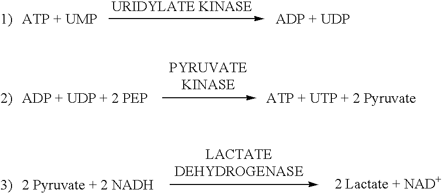

Abbreviations:

ATP/ADP—Adenosine 5′ triphosphate/Adenosine 5′-diphosphate

UMP/UDP—Uridine 5′ Monophosphate/Uridine 5′ diphosphate

PEP—Phospho(enol)pyruvate

NADH/NAD⁺—Nicotinamide Adenine Dinucleotide Reduced/Oxidized

The uridylate kinase coupled assay contains the following reagents in a final volume of 200 μl: assay buffer (50 mM HEPES, pH 7.5, 100 mM KCl, 2 mM MgCl₂), 1 mM UMP, 2 mM ATP, 0.22 mM NADH, 2 mM PEP, 3.2 units pyruvate kinase (Sigma, St. Louis, Mo.), 4 units lactate dehydrogenase, (Sigma) and 10 ng uridylate kinase. The assay was carried out at 25° C. with the decrease in absorbance at 340 nm monitored at 15 second intervals in the kinetics mode for 1 or 3 hours.

The complete disclosures of all patents, patent applications, publications, and nucleic acid and protein database entries, including for example GenBank accession numbers and EMBL accession numbers, that are cited herein are hereby incorporated by reference as if individually incorporated. Various modifications and alterations of this invention will become apparent to those skilled in the art without departing from the scope and spirit of this invention, and it should be understood that this invention is not to be unduly limited to the illustrative embodiments set forth herein.

### Sequence Listing Free Text

| | |
|---|---|
| SEQ ID NO:35-68, 90-108, 111, 112, 115, | Oligonucleotide primer |
| 116, 119, 120, 123, 124,127, 128, 131, 132, | |
| 135, 136, 139, 140, 143-145 | |
| SEQ ID NO:69, 71, 73, 75, 77, 79, 81, 83, | Nucleotide sequence of S. |
| 85 | aureus coding region |
| | cloned for expression in E. |
| | coli |
| SEQ ID NO: 70, 72, 74, 76, 78, 80, 82, 84, | Amino acid sequence |
| 86 | encoded by S. aureus |
| | coding region cloned for |
| | expression in E. coli |
| SEQ ID NOs:49, 53, 57, 61, 65, 69, 73, 77, | Cloned coding |
| 81, 85, 89, 93; | sequence |
| SEQ ID NOs:50, 54, 58, 62, 66, 70, 74, 78, | Polypeptide |
| 82, 86, 90, 94; | encoded by cloned |
| | essential coding |
| | sequence |
| SEQ ID NO:89 | DNA sequence of portion |
| | of pQE-60 vector |

What is claimed is:

1. A method for identifying an agent that binds a polypeptide, the method comprising:
contacting a polypeptide and an agent to form a mixture, wherein the polypeptide is encoded by a coding sequence comprising a nucleotide sequence selected from the group consisting of SEQ ID NO:7, 21, 23, 25, 27, 29, 31, 33, 109, 113, 117, 121, 125, 129, 133, 137, and 141;
determining whether the agent binds the polypeptide.

2. The method of claim 1 wherein determining comprises an assay selected from the group consisting of an enzyme assay, a binding assay, and a ligand binding assay.

3. The method of claim 1 further comprising determining whether the agent decreases the growth rate of a microbe, comprising:
contacting a microbe with the agent;
incubating the microbe and the agent under conditions suitable for growth of the microbe that is not contacted with the agent; and
determining the growth rate of the microbe contacted with the agent, wherein a decrease in growth rate compared to the microbe that is not contacted with the agent indicates the agent decreases the growth rate of the microbe.

4. The method of claim 3 wherein the microbe is in vitro or in vivo.

5. The method of claim 3 wherein the microbe is a Staphylococcus aureus.

6. An agent identified by the method of claim 1.

7. A method for identifying an agent that binds a polypeptide, the method comprising:
contacting a polypeptide and an agent to form a mixture, wherein the polypeptide is encoded by an essential coding sequence comprising a nucleotide sequence having at least about 57 percent structural similarity with a nucleotide sequence selected from the group consisting of SEQ ID NO:7, 21, 23, 25, 27, 29, 31, 33, 109, 113, 117, 121, 125, 129, 133, and 137;
determining whether the agent binds the polypeptide.

8. The method of claim 7 wherein determining comprises an assay selected from the group consisting of an enzyme assay, a binding assay, and a ligand binding assay.

9. The method of claim 7 further comprising determining whether the agent decreases the growth rate of a microbe, comprising:
contacting a microbe with the agent;
incubating the microbe and the agent under conditions suitable for growth of the microbe that is not contacted with the agent; and
determining the growth rate of the microbe contacted with the agent, wherein a decrease in growth rate compared to the microbe that is not contacted with the agent indicates the agent decreases the growth rate of the microbe.

10. The method of claim 9 wherein the microbe is in vitro or in vivo.

11. The method of claim 9 wherein the microbe is an S. aureus.

12. An agent identified by the method of claim 9.

13. A method for identifying an agent that binds a polypeptide, the method comprising:
contacting a polypeptide and an agent to form a mixture, wherein the polypeptide is encoded by a critical coding sequence comprising a nucleotide sequence having at least about 57 percent structural similarity with a nucleotide sequence comprising SEQ ID NO:141;
determining whether the agent binds the polypeptide.

14. The method of claim 13 wherein determining comprises an assay selected from the group consisting of an enzyme assay, a binding assay, and a ligand binding assay.

15. The method of claim 13 further comprising determining whether the agent decreases the growth rate of a microbe, comprising:
contacting a microbe with the agent;
incubating the microbe and the agent under conditions suitable for growth of the microbe that is not contacted with the agent; and
determining the growth rate of the microbe contacted with the agent, wherein a decrease in growth rate compared to the microbe that is not contacted with the agent indicates the agent decreases the growth rate of the microbe.

16. The method of claim 13 wherein the microbe is in vitro or in vivo.

17. The method of claim 13 wherein the microbe is an S. aureus.

18. An agent identified by the method of claim 13.

19. A method for identifying an agent that decreases the growth rate of a microbe, the method comprising:
contacting a microbe with an agent, wherein the agent binds to a polypeptide encoded by a coding sequence comprising a nucleotide sequence selected from the group consisting of SEQ ID NO:7, 21, 23, 25, 27, 29, 31, 33, 109, 113, 117, 121, 125, 129, 133, 137, and 141;
incubating the microbe and the agent under conditions suitable for growth of the microbe that is not contacted with the agent; and
determining the growth rate of the microbe contacted with the agent, wherein a decrease in growth rate compared to the microbe that is not contacted with the agent indicates the agent decreases the growth rate of the microbe.

20. The method of claim 19 wherein the microbe is in vitro or in vivo.

21. The method of claim 19 wherein the microbe is an S. aureus.

22. An agent identified by the method of claim 19.

23. A method for identifying an agent that decreases the growth rate of a microbe, the method comprising:
contacting a microbe with an agent, wherein the agent binds to a polypeptide encoded by an essential coding sequence comprising a nucleotide sequence having at least about 57 percent structural similarity with a nucleotide sequence selected from the group consisting of SEQ ID NO:7, 21, 23, 25, 27, 29, 31, 33, 109, 113, 117, 121, 125, 129, 133, and 137;
incubating the microbe and the agent under conditions suitable for growth of the microbe that is not contacted with the agent; and
determining the growth rate of the microbe contacted with the agent, wherein a decrease in growth rate compared to the microbe that is not contacted with the agent indicates the agent decreases the growth rate of the microbe.

24. The method of claim 23 wherein the microbe is in vitro or in vivo.

25. The method of claim 23 wherein the microbe is an S. aureus.

26. An agent identified by the method of claim 23.

27. A method for identifying an agent that decreases the growth rate of a microbe, the method comprising:
contacting a microbe with an agent, wherein the agent binds to a polypeptide encoded by a critical coding sequence comprising a nucleotide sequence having at least about 57 percent structural similarity with a nucleotide sequence comprising SEQ ID NO:141;
incubating the microbe and the agent under conditions suitable for growth of the microbe that is not contacted with the agent; and
determining the growth rate of the microbe contacted with the agent, wherein a decrease in growth rate compared to the microbe that is not contacted with the agent indicates the agent decreases the growth rate of the microbe.

28. The method of claim 27 wherein the microbe is in vitro or in vivo.

29. The method of claim 27 wherein the microbe is an S. aureus.

30. An agent identified by the method of claim 27.

31. A method for decreasing the growth rate of a microbe, the method comprising:
contacting a microbe with an agent that binds to a polypeptide encoded by a coding sequence comprising a nucleotide sequence selected from the group consisting of SEQ ID NO:7, 21, 23, 25, 27, 29, 31, 33, 109, 113, 117, 121, 125, 129, 133, 137, and 141.

32. The method of claim 31 wherein the microbe is in vitro or in vivo.

33. The method of claim 31 wherein the microbe is an S. aureus.

34. A method for decreasing the growth rate of a microbe, the method comprising:
contacting a microbe with an agent that binds to a polypeptide encoded by an essential coding sequence comprising a nucleotide sequence having at least about 57 percent structural similarity with a nucleotide sequence selected from the group consisting of SEQ ID NO:7, 21, 23, 25, 27, 29, 31, 33, 109, 113, 117, 121, 125, 129, 133, and 137.

35. The method of claim 34 wherein the microbe is in vitro or in vivo.

36. The method of claim 34 wherein the microbe is an S. aureus.

37. A method for decreasing the growth rate of a microbe, the method comprising:
contacting a microbe with an agent that binds to a polypeptide encoded by a critical coding sequence comprising a nucleotide sequence having at least about 57 percent structural similarity with a nucleotide sequence comprising SEQ ID NO:141.

38. The method of claim 37 wherein the microbe is in vitro or in vivo.

39. The method of claim 38 wherein the microbe is an S. aureus.

40. A method for making an S. aureus with reduced virulence, the method comprising:
altering a coding sequence in an S. aureus to comprise a mutation, the non-mutagenized coding sequence comprising a nucleotide sequence selected from the group consisting of SEQ ID NO:7, 21, 23, 25, 27, 29, 31, 33, 109, 113, 117, 121, 125, 129, 133, 137, and 141; and
determining if the S. arueus comprising the mutation has reduced virulence compared to an S. arueus that does not comprise the mutation.

41. An S. aureus of claim 40.

42. A vaccine composition comprising the S. aureus organism of claim 40.

43. A method for making an S. aureus with reduced virulence, the method comprising:
altering an essential coding sequence in an S. aureus to comprise a mutation, the non-mutagenized coding sequence comprising a nucleotide sequence having at least about 57 percent structural similarity to a nucleotide sequence selected from the group consisting of SEQ ID NO:7, 21, 23, 25, 27, 29, 31, 33, 109, 113, 117, 121, 125, 129, 133, and 137; and
determining if the S. arueus comprising the mutation has reduced virulence compared to an S. arueus that does not comprise the mutation

44. An S. aureus of claim 43.

45. A vaccine composition comprising the S. aureus organism of claim 43.

46. A method for making an S. aureus with reduced virulence, the method comprising:
altering a critical coding sequence in an S. aureus to comprise a mutation, the non-mutagenized coding sequence comprising a nucleotide sequence having at least about 57 percent structural similarity to a nucleotide sequence comprising SEQ ID NO:141; and
determining if the S. arueus comprising the mutation has reduced virulence compared to an S. arueus that does not comprise the mutation

47. An S. aureus of claim 46.

48. A vaccine composition comprising the S. aureus organism of claim 46.

49. An isolated polynucleotide comprising a nucleotide sequence selected from the group consisting of SEQ ID NO:7, 21, 23, 25, 27, 29, 31, 33, 109, 113, 117, 121, 125, 129, 133, 137, and 141.

50. An isolated polynucleotide consisting essentially of a nucleotide sequence selected from the group consisting of SEQ ID NO:7, 21, 23, 25, 27, 29, 31, 33, 109, 113, 117, 121, 125, 129, 133, 137, and 141, and wherein the polynucleotide optionally further comprises from zero to up to about 5,000 nucleotides upstream and/or downstream of the nucleotide sequence selected from the group consisting of SEQ ID NO:7, 21, 23, 25, 27, 29, 31, 33, 109, 113, 117, 121, 125, 129, 133, 137, and 141.

51. An isolated polynucleotide comprising a nucleotide sequence having at least about 57 percent structural similarity with a nucleotide sequence selected from the group consisting of SEQ ID NO:7, 21, 23, 25, 27, 29, 31, 109, 113, 117, 121, 125, 129, 133, and 137, wherein the isolated polynucleotide comprises an essential coding sequence.

52. An isolated polynucleotide consisting essentially of a nucleotide sequence having at least about 57 percent structural similarity with a nucleotide sequence selected from the group consisting of SEQ ID NO:7, 21, 23, 25, 27, 29, 31, 109, 113, 117, 121, 125, 129, 133, and 137, wherein the isolated polynucleotide comprises an essential coding sequence.

53. An isolated polynucleotide comprising a nucleotide sequence having at least about 57 percent structural similarity with a nucleotide sequence comprising SEQ ID NO:141, wherein the isolated polynucleotide comprises a critical coding sequence.

54. An isolated polynucleotide consisting essentially of a nucleotide sequence having at least about 57 percent structural similarity with a nucleotide sequence comprising SEQ ID NO:141, wherein the isolated polynucleotide comprises a critical coding sequence.

55. An isolated polypeptide comprising an amino acid sequence selected from the group consisting of SEQ ID NO:2, 4, 6, 8, 10, 12, 14, 16, 18, 20, 22, 24, 26, 28, 30, 32, 34, 110, 114, 118, 122, 126, 130, 134, 138, and 142.
